(19) United States
(12) Patent Application Publication
(10) Pub. No.: US 2024/0368626 A1
(43) Pub. Date: Nov. 7, 2024

(54) T-CELL MANUFACTURING METHODS

(71) Applicant: SEATTLE CHILDREN'S HOSPITAL D/B/A SEATTLE CHILDREN'S RESEARCH INSTITUTE, Seattle, WA (US)

(72) Inventors: Michael C. Jensen, Bainbridge Island, WA (US); James Rosser, Bellingham, WA (US)

(73) Assignee: SEATTLE CHILDREN'S HOSPITAL D/B/A SEATTLE CHILDREN'S RESEARCH INSTITUTE, Seattle, WA (US)

(21) Appl. No.: 18/681,783

(22) PCT Filed: Aug. 5, 2022
(86) PCT No.: PCT/US22/74630

§ 371 Date: Feb. 6, 2024

Related U.S. Application Data
(60) Provisional application No. 63/230,644, filed on Aug. 6, 2021.

(51) Int. Cl. C12N 15/86; C07K 14/725; C12N 5/0783

(52) U.S. Cl. CPC C12N 15/86; C07K 14/7051; C12N 5/0636; C12N 2740/15043

(57) ABSTRACT

T cell manufacturing methods that co-culture T cells with autologous cell types to stimulate the T cells during manufacture are described. The T cells express a recombinant receptor. A binding domain expressed by the T cells binds an epitope on stimulating autologous cell types and/or an immune cell activating multi-specific binding molecule (e.g., a bi-specific antibody) during the co-culture. The methods can also allow for the expression of large transgenes by utilizing electroporation and transposons to deliver transgenes encoding the recombinant receptor. The disclosed methods create manufactured T cell populations with a high number of cells in comparison to starting cell numbers, a high percentage of recombinant receptor-expressing T cells within the cell number, and a beneficial naïve T cell marker profile.

### CROSS-REFERENCE TO RELATED APPLICATIONS

This application is a US National Phase Patent Application based on International Patent Application No. PCT/US2022/074630, filed on Aug. 5, 2022, which claims priority to U.S. Provisional Patent Application No. 63/230,644 filed Aug. 6, 2021, each of which is incorporated herein by reference in their entirety as if fully set forth herein.

### REFERENCE TO SEQUENCE LISTING

The Sequence Listing associated with this application is provided in XML format in lieu of a paper copy and is hereby incorporated by reference into the specification. The name of the file containing the Sequence Listing is 44784629.xml. The file is 101,409 bytes, was created on Feb. 6, 2024, and is being submitted electronically via Patent Center.

### FIELD OF THE DISCLOSURE

The current disclosure provides T cell manufacturing methods that co-culture T cells with autologous cell types to stimulate the T cells during manufacture. The T cells express a recombinant receptor. A binding domain expressed by the T cells binds an epitope on stimulating autologous cell types and/or an immune cell activating multi-specific binding molecule (e.g., a bi-specific antibody) during the co-culture. The methods can also allow for the expression of large transgenes by utilizing electroporation and transposons to deliver transgenes encoding the recombinant receptor. The disclosed methods create manufactured T cell populations with a high number of cells in comparison to starting cell numbers, a high percentage of recombinant receptor-expressing T cells within the cell number, and a beneficial naïve T cell marker profile.

### BACKGROUND OF THE DISCLOSURE

A variety of therapies developed over the last few decades engineer T cells to express a recombinant receptor that binds an antigen on unwanted cell types (e.g., cancer cells, virally infected cells). When the recombinant receptor binds the target antigen, the T cell can be activated to destroy the bound cell type. Chimeric antigen receptor (CAR)-T cell therapy is one such therapy.

CAR-T cell therapy involves the engineering of T cells to express recombinant molecules that include a binding domain that binds an antigen on an unwanted cell type, a transmembrane domain, and a cell activation domain that activates the T cell upon binding of the binding domain to the antigen.

Currently, the standard manufacture of CAR-T cells uses lentiviral or gamma-retroviral transduction of T cells and subsequent activation of the transduced T cells to produce the desired population phenotype and size. Although this method has shown remarkable efficacy in clinical trials, challenges in the manufacture of such T cells remain. For example, obtaining a sufficient number of genetically modified cells remains a challenge, particularly when starting the manufacturing process with a limited cell number. The use of lentiviral or gamma-retroviral transduction raises the potential for insertional mutagenesis and the genotoxicity of viral vectors causes concern. Furthermore, viral transduction is limited by the size of deliverable DNA and larger constructs have reduced transfection efficiency. Each of these challenges limits more widespread clinical translation of this important therapeutic medicine. As such, there is a need for a method of T cell manufacture that results in a high fold expansion of clinical-grade T cells with high recombinant receptor expression positivity, a capacity for large transgene insertion, and a beneficial naïve cell marker profile.

### SUMMARY OF THE DISCLOSURE

The current disclosure provides methods to produce a population of recombinant receptor-expressing cells with a high cell count and high recombinant receptor positivity within the cell count. The methods also allow the capacity for large transgene insertion, and a beneficial naïve cell marker profile.

In particular embodiments, the methods include obtaining T cells positive for a marker from a sample, retaining the negative fraction of the sample, modifying obtained T cells to express a recombinant receptor, and culturing the recombinant receptor-expressing T cells within the negative fraction, wherein a binding domain expressed by the recombinant receptor-expressing T cells binds an immune cell activating epitope within the culture. In particular embodiments, the recombinant receptor binds the immune cell activating epitope within the culture. In particular embodiments, the immune cell activating epitope is expressed by an autologous immune cell within the negative fraction and/or is a binding domain on a multi-specific binding molecule.

In particular embodiments, examples of these methods include:
obtaining CD8+ cells isolated from a sample;
obtaining CD4+ cells isolated from the same sample;
retaining the sample following the CD8+ cell isolation and the CD4+ cell isolation (the negative fraction);
introducing a chimeric antigen receptor (CAR)-encoding genetic construct into the isolated CD8+ T cells, wherein the CAR-encoding genetic construct results in expression of a CAR by the CD8+ T cells, and wherein the CD8+ T cells express a binding domain that binds an immune cell activating epitope; and
co-culturing the CAR-expressing CD8+ cells with the negative fraction within a cytokine-supplemented media.

In particular embodiments, the co-culturing includes co-culturing the CAR-expressing CD8+ cells with the isolated CD4+ cells and the negative fraction within a cytokine-supplemented media.

In other embodiments, the methods include:
obtaining CD8+ cells isolated from a sample;
obtaining CD4+ cells isolated from the same sample;
retaining the sample following the CD8+ cell isolation and the CD4+ cell isolation (the negative fraction);
introducing a CAR-encoding genetic construct into the isolated CD4+ T cells, wherein the CAR-encoding genetic construct results in expression of a CAR by the CD4+ T cells, and wherein the CD4+ T cells express a binding domain that binds an immune cell activating epitope; and
co-culturing the CAR-expressing CD4+ cells with the negative fraction within a cytokine-supplemented media.

In particular embodiments, the co-culturing includes co-culturing the CAR-expressing CD4+ cells with the isolated CD8+ cells and the negative fraction within a cytokine-supplemented media.

In other embodiments, the methods include:
obtaining CD8+ cells isolated from a sample;
retaining the sample following the CD8+ cell isolation (the negative fraction);
introducing a CAR-encoding genetic construct into the isolated CD8+ T cells, wherein the CAR-encoding genetic construct results in expression of a CAR by the CD8+ T cells, and wherein the CD8+ T cells express a binding domain that binds an immune cell activating epitope; and
co-culturing the CAR-expressing CD8+ cells with the negative fraction within a cytokine-supplemented media.

In other embodiments, the methods include:
obtaining CD4+ cells isolated from a sample;
retaining the sample following the CD4+ cell isolation (the negative fraction);
introducing a CAR-encoding genetic construct into the isolated CD4+ T cells, wherein the CAR-encoding genetic construct results in expression of a CAR by the CD4+ T cells, and wherein the CD4+ T cells express a binding domain that binds an immune cell activating epitope; and
co-culturing the CAR-expressing CD4+ cells with the negative fraction within a cytokine-supplemented media.

In particular embodiments, the immune cell activating epitope is a portion of a molecule (e.g., a portion of a protein) expressed by an autologous immune cell within the negative fraction. In particular embodiments, the immune cell activating epitope is a binding domain on a multi-specific binding molecule (e.g., bi-specific antibody). In either or both of these embodiments, the binding domain that binds the immune cell activating epitope can be the binding domain of the CAR.

In certain examples, the cytokine-supplemented media includes interleukin (IL)-4, IL7, and IL21. These cytokines can be present in concentrations of, for example, 20 ng/ml IL4, 10 ng/ml IL7, and 20 ng/ml IL21.

In certain examples, the cytokine-supplemented media includes interleukin (IL)-2, IL4, IL7, and IL21. These cytokines can be present in concentrations of, for example, 50 U/mL IL2, 20 ng/ml IL4, 10 ng/ml IL7, and 20 ng/ML IL21.

In certain examples, the ratio of CD8+ cells to CD4+ cells to the negative fraction of cells during a co-culture is 1:1:1, 1:1:2, 1:1:3, 1:1:4, 2:1:1, 3:1:2, or 3:2:4. In certain examples, the ratio of CD8+ cells to CD4+ cells to the negative fraction of cells during a co-culture is 1:1:1, 1:1:2, 1:1:3, 1:1:4, 2:1:1, 3:1:2, or 3:2:4. In certain examples, the introducing of the recombinant receptor-encoding construct into a T cell (e.g., CD8+ T cell or CD4+ T cell) is through electroporation. Introduction of recombinant receptor-encoding constructs can include introduction of plasmid DNA and a transposase. In particular embodiments, the recombinant receptor is a CAR. The CAR-encoding construct can be 15 kb in size.

The methods can further include a recombinant receptor-expressing T cell enrichment step to purify the cell population.

Practicing the presently-disclosed methods, recombinant receptor-expressing T cell populations with greater than 80% and greater than 90% of T cells expressing the recombinant receptor were obtained. Further, over 75 million recombinant receptor-expressing T cells were obtained following introduction of the genetic constructs into only 2 million cells. In certain examples, over 100 million recombinant receptor-expressing T cells were obtained following introduction of the genetic constructs into only 2 million cells. Further, the expanded recombinant receptor-expressing T cell populations retained favorable more naïve cell marker profiles, such as CD62L+/CD45RA+and CD27+/CD28+.

The disclosed methods thus provide an important advance in T cell manufacturing by providing a high cell count, high percentage of recombinant receptor positivity within the cell count, the capacity for large transgene insertion, and a beneficial naïve cell marker profile.

### BRIEF DESCRIPTION OF THE SEVERAL VIEWS OF THE DRAWINGS

Some of the drawings submitted herein may be better understood in color. Applicant considers the color versions of the drawings as part of the original submission and reserves the right to present color images of the drawings in later proceedings.

FIG. 1. Workflow for methods disclosed herein using an electroporation format. CD8+, CD4+, and the negative fraction of Leukopak are isolated using an MM24 (Miltenyi Biotec, Bergisch Gladbach, Germany) protocol. The negative fraction cells, depleted of CD8+ and CD4+ cells, are frozen in liquid nitrogen until initiation of re-introduction of CAR-modified cells, and, depending on the protocol, other supporting cells. Here, CD8+ cells, CD4+ cells and the negative fraction including PBMCs (referred to hereafter as negative selection PBMCs (nsPBMCs)) are thawed, and briefly rested. CD8+ cells were electroporated with 10 mM plasmid DNA, 40 mM transposase in either stripwell or cuvette format using the EO-115 Lonza (Basel, Switzerland) protocol. After electroporation, electroporated CD8+ cells, the isolated CD4+ cells, and the nsPBMCs are seeded into G-REX® (Wilson Wolf Corporation, New Brighton, MN) at a CD8+:CD4+:nsPBMC ratio of 1:1:3 in X-VIVO™ 15 (Lonza, Basel, Switzerland)+2% KnockOut™ Serum Replacement (KO) (Thermo Fisher Scientific, Waltham, MA) with 50 U/mL IL2, 20 ng/ml IL4, 10 ng/ml IL7 and 20 ng/ml IL21. Cells are fed every 3-4 days with media changes and the culture vessel is scaled up to not exceed the vessel's maximum carrying capacity. Expansion ends at 21 days. Cells are checked for purity via flow cytometry and cryopreserved for further use. EP stands for electroporated. Within the FIGS., TICLE refers to a process wherein CAR-expressing cells are stimulated during co-culture with other cell types (e.g., CAR-expressing CD8+ cells are stimulated during co-culture with nsPBMCs (e.g., B cells within the nsPBMCs) and CD4+ cells).

FIG. 2. Electroporation with pJ04052—Day 14. Using pJ04052 (CD19scFv-IgG4 hinge-CD28tm-41BB-CD3Zeta-T2A-EGFRt-T2A-DHFRdm_PiggyBac Nanoplasmid (PBNP)), a 5kb PBNP construct was electroporated with piggybac (PB) transposase RNA in CD8+ T cells. CD4+ T cells and nsPBMCs from the same donor were added back at different ratios (1:0, 2:1, 1:1 and 1:2) to the electroporated CD8+ cells and cultured in IL2, IL7, IL15 and IL21 or IL2, IL4, IL7, and IL21. Unless otherwise noted, cytokine concentration are as in FIG. 1. IL15 was used at 0.5 ng/ml. Additionally, a set of conditions were initiated where only autologous CD4+ cells were added back to the culture; this set of conditions was subjected to a soluble CD3/CD28 stimulation reagent (TransAct, Miltenyi) and cultured in II2, II7, IL15 and IL21. Further, half of culture conditions were subjected to methotrexate (50 nM) selection for 10 days. After 14 days, only CD4+/nsPBMC addback cultured in IL2, IL4, IL7, and IL21 supported the expansion of the CD8+ population. Selection with methotrexate further enriches the CD8+ population. Other culture conditions were not able to support the expansion of the CD8+ population. Addback ratios of CD4+ cells to nsPBMC of 1:1 and 1:2 favor expansion. Values in top right corner of chart indicate CD8+CD4− cells and value at bottom of chart indicate CD4+ CD8− cells.

FIG. 3. Electroporation with pJ04052—Day 14. EGFRt was used to determine the level of CD19CAR positivity for the various culture conditions previously described. EGFRt positivity was substantially enhanced in the presence of CD4+/nsPBMC addback cultured in IL2, IL4, IL7, and IL21. Further, selection with 50 nM methotrexate further purified the CAR-expressing cells within the cell population to over 90 percent. Unless otherwise specified, references to CD19CAR within these FIGS. descriptions refer to pJ04052.

FIGS. 4. After 21 days of expansion, cells are highly positive for EGFRt even in the absence of methotrexate selection. CD8+ cells were electroporated with either a CD19CAR-DHFRdm-EGFRt (pJ04052-5kb) or no DNA (Mock). Cells were cultured in the presence of autologous CD4+/nsPBMCs and expanded in X-VIVO™ (Lonza)+2% KO plus IL2, IL4, IL7, and IL21 for 21 days. Further to serve as control conditions, electroporation of CD8s with mock or a CD19CAR-DHFRdm-EGFRt (pJ04052-5kb) PBNP construct was initiated with a CD8: PBMC/CD4 addback ratio 1:1 but cultured in IL2, IL7, IL15 and IL21 and expanded for 21 days. Flow cytometry analysis for cells gated on CD3+, CD8+ and Erbitux show a strong enrichment in EGFRt positive cells after the end of expansion when cultured in the presence of CD4+/nsPBMC addback and IL2, IL4, IL7, and IL21. While enrichment of EGFRt in CD8 s was obtained using IL2, IL7, IL15 and IL21, total cell expansion was not comparable to expansion with IL2, IL4, IL7 and IL21 (data not shown). Note that high CAR+purity was obtained without methotrexate selection.

FIG. 5. Compatibility with large, complex constructs that carry additional payloads. Constructs in the PBNP format that are more complex and larger than the standard CD19CAR-encoding construct used (top construct schema) were processed according to the protocol depicted in FIG. 1 using CD8+ cells and expanded for 21 days. These constructs all carry the same CD19CAR with selection gene DHFRdm and detection marker EGFRt. Upstream contains an inducible (iSynPro) or constitutive (MND) promoter driving eGFP: ffluc or a CAR modifying payload.

FIG. 6. CAR specific expansion results in high purity and cell yield after culture in X-VIVO™ 15 (Lonza)+2% KO with IL2, IL4, IL7 and IL21 (cytokine concentrations as in FIG. 1). CD8+ cells were electroporated using constructs that varied in size and complexity and expanded for 21 days. Constructs included a CD19CAR and several dual promoter constructs that varied in promoter and payload types. Total live cells for all conditions expanded from 2×10⁶ CD8+ cells to over 100×10⁶ CD8+ cells. In conditions with CD8-specific electroporation of CD19CAR, most CD8+ conditions expanded to 80% purity. The purity of EGFRt+ cells in CD8+ cells was 80% and above for most conditions. In total, CD8+EGRt+ cell number yielded between 50 to 100×10⁶ cells.

FIG. 7. Methotrexate selection compatibility. Constructs that contain DHFRdm are candidates for methotrexate selection to further purify the CAR positive CD8+ fraction being expanded. CD8+ cells were electroporated and 50 nM methotrexate was added to the culture at Day 3. Cells were expanded until Day 21 according to the protocol of FIG. 1 with a cell addback ratio of 1:2 and were characterized using flow cytometry. These results suggest compatibility with methotrexate selection and an expansion benefit to CD8+CAR+ T cell.

FIG. 8. IL2 promotes stronger expansion. The addition of IL2 in addition to IL4, IL7 and IL21 supports a stronger expansion of CD8+ CAR positive cells compared to the cytokine cocktail of IL4, IL7 and IL21. CD8+ cells were electroporated and processed as described in relation to FIG. 1, but in the presence or absence of IL2. The total number of cells and cell phenotype were determined at Day 21.

FIG. 9. IL2 does not affect the CD62L+ CD45RA+ population. The addition of IL2 in addition to IL4, IL7 and IL21 does not negatively affect the expression of CD62L+ and CD45RA+ markers. CD45RA+ and CD62L+ cells are indications of a naïve phenotype.

FIG. 10. The addition of IL2 in addition to IL4, IL7, and IL21 does not negatively affect the expression of CD27+ and CD28+ markers. CD27+ and CD28+ markers are indications of naïve T cell phenotype.

FIG. 11. CAR-specific stimulation to make individual lines. CD8+ cells were electroporated with a suite of constructs with differing payloads and processed according to the protocol of FIG. 1. Each line was carried separately and characterized for CD8+ and EGFRt purity. All lines are high in CD8+CD19CAR purity.

FIG. 12. At the end of manufacture according to the protocol of FIG. 1, CD8+ cells are highly enriched for CD19CAR. However, the carried payload is in the off state and is not expressed.

FIG. 13. Phenotypic analysis of CD8+ cells using CD45RA and CD62L. Following manufacture according to the protocol of FIG. 1, most lines are positive for CD45RA and CD62L, markers that are associated with Tn and Tscm (CD45RA+,CD62L+,CD27+,CD62L+) phenotypes.

FIG. 14. Chromium Release Assay (CRA). CD19CAR+CD8+ cells (test) were functionally benchmarked against the same CD19CAR construct produced using standard lentiviral transduction (control) in a CRA. In relation to this and the following FIGS., “test” refers to processed according to the protocol of FIG. 1 Effector and chromium 51 labeled targets were cocultured for 4 hours at different E:T ratios (30:1, 10:1, 3:1, and 1:1). Following, the amount of chromium 51 released was quantified indicative of the target cell lysis that occurred. Test constructs (starred lines) demonstrated the ability to lyse targets when antigen was present and was comparable to control. There may be more EP CAR effector cells compared to all other effectors due to pipetting error. CRA represents qualitative analysis; it gives a result of whether the CAR is functional, but differences in the degree of lysis are minor (4-hour incubation only). Mock lines are not cytotoxic against CD19+ lines. Test CD8+ CAR+ cells show killing of target in appropriate manner.

FIG. 15. Intracellular Cytokine Staining (ICCS). Test and control (virally-transduced) CD8+ cells were subjected to an ICCS assay to functionally qualify the test method. Briefly, effectors and targets were cocultured for 4 to 5 hours. Following, intracellular cytokine staining was performed to determine the level of 4-1BB, Nur77 and CD107a. Test CAR cells express more 4-1BB than lentivirus (LV) control CAR in the presence of CD19 antigen. Test CAR cells express a similar level of Nur77 as LV control CAR cells. Test CAR cells express more CD107a than LV control CAR after stimulation with targets expressing CD19 or OKT3. Test CD8+ CAR+ cells increase expression of activation markers in appropriate conditions. Differing stimulation conditions were tested including no stimulation (Unstimulated), PMA/ionomycin, Raji, K562, K562 CD19 and K562 OKT3. PMA/ionomycin causes TCR-independent stimulation. Raji is a lymphoma line expressing CD19. K562 is a myelogenous leukemia cell line. K562 CD19 expresses a truncated from a CD19t that is recognized by CD19CAR T cell. K562 OKT3 causes all T cells to get activated.

FIG. 16. IncuCyte Cytotoxicity Assay. An Incucyte killing assay reveals that Test CD19CAR+ CD8+ cells can effectively kill and eliminate targets compared to traditionally prepared CD8+ CD19CAR positive cells produced through lentiviral transduction (control). Effector and mCherry-labeled targets were set up in a coculture killing assay at a 1:1 ratio and the level of mCherry was determined over 5 days. Test and lentiviral-transduced control CD19CAR CD8+ cells were able to control targets when the CD19 antigen was present indicating that Test CD8+ cells are functionally comparable. This FIG. shows a 1:1 E:T ratio for simplicity. Targeted killing is observed in all Test effector groups containing CAR, suggesting similar functional ability compared to traditional lentiviral-based CAR T-cells.

FIG. 17. The methods disclosed herein are compatible with CD4 specific electroporation and allow for the same benefits seen in a CD8+ specific Test: high yield and purity of CD4+CD19CAR+ cells. CD4+ cells were electroporated with either a human CD19CAR construct or a larger, more complex human CD19CAR construct containing a CAR-modifying transgene (PD1 (A99L): MYD88) under the regulation of an inducible promoter (iSynPro). The human CD19 CAR, referred to as pJ05591, includes EF1a (I) _huCD19scFv (G01S)-IgG4hinge-CD28tm-41BB-CD3zeta-P2A-DHFRdm-T2A-EGFRt_PBNP. Electroporated cells were expanded using the same methodology developed for the CD8-specific Test as depicted in FIG. 1. After expansion, CD4 cells showed high purity for CD19CAR for the human CD19CAR construct and the construct containing a human CD19CAR and an inducible CAR modifying transgene (PD1 (A99L): MYD88).

FIG. 18. Scalability. CD4+ or CD8+ cells from two different donors (D1 or D2) were electroporated with various constructs that varied in size and complexity at a larger scale (10×10⁶ CD4+ or CD8+ cells electroporated versus a standard size of 2×10⁶ cells). Cells were cultured in a G-REX® (Wilson Wolf Corporation) format for 21 days as shown in FIG. 1 (altered according to whether CD4+ or CD8+ cells were electroporated or part of the addback population) and cell counts were taken to determine total cell expansion.

FIG. 19. High purity CD8+ cells that are high purity CAR. CD8+ cells that were processed at a higher scale were phenotypically characterized using flow cytometry and were shown to be highly enriched for CD19CAR as indicated by EGFRt expression in CD8+ cells. The low Her2tG expression shows that the inducible promoter is in an off state. pJ04788 (S1-61-IL2mp_PD1 (A99L): MYD88-T2A-Her2tG_EF1a (I)_huCD19scFv (G01S)-IgG4hinge-CD28tm-41BB-CD3zeta-P2A-DHFRdm-T2A-EGFRt_PBNP).

FIG. 20. CD4+ or CD8+ purify and are EGFRt+. CD4+ cells that were processed at a higher scale were phenotypically characterized using flow cytometry and were shown to be highly enriched for CD19CAR as indicated by EGFRt expression in CD4+ cells and CD8+ cells. Cells were also characterized for GFP to determine the level of expression that originated from the inducible promoter. A majority of the cell population resides in the off state.

FIG. 21. Unstimulated PBMCs containing CD4+ and CD8+ cells were plated into a G-REX® (Wilson Wolf Corporation) in X-VIVO™ 15 (Lonza) with 2% KO and IL2, IL4, IL7, and IL21. Lentivirus containing either a vector expressing a CD19CAR or a CD33CAR were used to transduce unstimulated cells at a multiplicity of infection (MOI) of 10, 5, or 1. The CD19 CAR included CD19R-IgG4 Hinge only-CD28tm-41BB-Zeta-T2A-EGFRt_epHIV7 and the CD33 CAR included CD33 h2H12 VH VL-IgG4 hinge-CD28tm/41BB-zeta-T2A-EGFRt-epHIV7.2. As a control, each CAR virus was used at a MOI of 1 to transduce PBMCs that were TransAct (Milteyni) stimulated (TA stim) and grown in either IL2, IL4, IL7 and IL21 or IL7, IL15 and IL21. Cells were expanded in a G-REX® (Wilson Wolf Corporation) and cultured for 14 days. Cell phenotypes were assessed by flow cytometry and show high purity of EGFRt+ CD4+ or CD8+ cells for both CD19CAR and CD33CAR. Higher MOI enhances the level of purity after the unstimulated viral processing. These data suggest compatibility with an unstimulated transduction and multiple CAR formats.

FIG. 22. CD19CAR (PBNP) vs CD19CAR (sinSB). The method disclosed herein is amenable to producing T cells modified with CD19CAR though orthogonal means of vector delivery. CD19CAR harbored a piggyBac nanoplasmid vector (PBNP) plus piggyBac transposase or a vector containing a self-inactivating Sleeping Beauty transposase and a CD19CAR flanked by Sleeping Beauty inverted terminal repeats. These were electroporated into T cells and CAR positive cells were selectively stimulated and expanded using the method disclosed herein. Cells were assessed for EGFRt through flow cytometry to indicate CAR positivity. In two donors EGFRt positive cells were obtained using the self-inactivating Sleeping Beauty transposase system showing this vector architecture is compatible.

FIG. 23. RNAseq analysis of PD-like, viral-modified, and electroporated cells. CD19CAR can be introduced into T cells through several different means. Classically, T cells are stimulated with CD3/28 beads and transduced with CAR vector+ virus (PD-like process). The methods disclosed herein were developed to be conducive to both electroporation (ep) and viral (viral) formats. Here, the production endpoints of these three production methods were compared to understand the functional differences related to the T cell transcriptome. T cells were modified with CD19CAR using the PD-like process (CD3/28 stim), a viral-introduced process or an electroporation-introduced process and cultured to D14. After 14 days, cells were unstimulated or stimulated with TransAct, a soluble CD3/28 stim for 16 hr and cells were processed for RNA seq. Principal Component Analysis shows transcriptional profiles between the PD-like process and a viral-introduced process to be similar while the electroporation-introduced process clustered away from the other two production methods.

FIG. 24. A chemical adaptor can enhance expansion of a solid tumor CAR antigen. CARs targeting solid tumor require their cognate antigen to be expressed on the PBMC population to provide a robust stimulating for subsequent expansion. The aim of this experiment was to show that the methods are not restricted to antigens that are robustly present on PBMCs by providing a chemical adaptor to crosslink the solid tumor CAR to a cell within the PBMCs population. To achieve this, a human Her2-Fc conjugate was used to link the HER2CAR to cells expressing Fc receptors in the PBMC population. The methods described herein were initiated as normal except 10 ug of human Her2-Fc conjugate was added to the “w/adaptor ep” conditions. 24 hours after initiation, another 10ug of adaptor was added. For the Her2 CAR, cell expansion was suboptimal compared to the gold standard CD19CAR. However, when the Her2-Fc intermediate was added, cell expansion of EGFRt+ (CAR positive) cells was greatly enhanced.

FIGS. 25A, 25B. Schematic representing (25A) standard method and the (25B) select method. The select method is a high throughput and selective CAR T Cell expansion protocol for complex CAR and drug resistant gene expressing constructs. The select method is a method of delivering desired payload combinations into CAR T cell systems. Ideally, only the cells that have received both plasmids (dual EP'd) will become the dominant population in culture. The cells are enriched via CAR stimulation (expansion) and must survive MTX selection (selection). Using separate plasmids provides multiple avenues for delivering other payloads, it is highly modular, and can use various combinations of potency enhancers (TgX) and/or CARs.

FIG. 26. The select method was tested with simple plasmids and following the pictured timeline. The control plasmid pJ05591: EF1a (L) huCD19scFv (G01S)-IgG4hinge-CD28tm-4-1BB-zeta-P2A-DHFRdm-T2A-EGFRt_PBNP (5366 bp). The experimental plasmids are: pJ06402: EF1a (L) _DHFRdm_P2A_Her2tG_RBGPolyA_PBNP (3291 bp); pJ06403: EF1a (L) _huCD19scFv (G01S)-IgG4hinge-CD28tm-41BB-zeta-T2A-EGFRt_RGBpolyA_PBNP (4748 bp); and Dual EP (1:1):pJ06402 (5 nM)+pJ06403 (5 nM).

FIGS. 27A-27D. Donor B T1D21. In (27A) Donor B CD4+ T1D21 and (27B) Donor B CD8+ T1D21 cells, constructs related to the select method were subjected to ep, cultured for 21 days and assessed for EGFRt and Her2tG positivity by flow cytometry to determine the number of cells that harbored integrants containing a CD19CAR and the DHFRdm cassette. Cells were subjected to no selection or selection with 50 nM methotrexate (MTX). A higher level of EGFRt and Her2tG positivity was achieved when T cells were subjected to methotrexate selection. Treating cultures with MTX reduces single positive populations and enriches the target dual positive population. (27C, 27D) The yield for dual EP condition containing the CD19CAR and DHFRdm split across two vectors is comparable to “all in one” condition where both cassettes are harbored on one vector for (27C) CD4 and (27D) CD8 cells.

FIG. 28. Cell yield at end of production. The select method as applied to larger, complex construct containing CD19CAR and a CAR inducible promoter S1-61 (iSynPro) driving a marker turboGFP. After expansion, cell yields for dual EP'd cells are comparable or higher than the “all-in-one” construct produced cells in CD4+ and CD8+ CAR T cells.

FIG. 29. CD4 addback and IL-2 supplementation does not increase cell yields. The role of IL2 related to supporting cell expansion was explored in a CD4 or CD8 electroporation-introduced process. CD19CAR was introduced to CD4s or CD8s through the means of piggyBac transposition via electroporation and cultured for 14 days in media with or without IL2. Further, the role of CD4 support (+AB; additional CD4s being added to the culture) for a CD8 specific electroporation-introduced process was explored. No appreciable difference in cell expansion was detected after 14 days in conditions containing IL2 or CD4 addback (+AB) suggesting flexibility in the process when reagents are limited.

FIG. 30. FL CAR expression. The method is compatible when using a hapten specific CAR. To explore the compatibility of using a CAR against a non-native protein, donor nsPBMCs were labeled with 100% or 10% of fluorescein (FL) phospholipid ether (FL-PLE), a hapten-lipid conjugate that can be administered which intercalates into cell membranes and is reactive to FL-targeting CAR T cells. FL-CAR were generated via transposon-based methods as described earlier and incubated with FL-PLE labeled nsPBMCs and tracked via CD19t, a marker of cell modification. Cell counts were determined to assess overall cell expansion. A high purity of cells was achieved after expansion and cell counts suggest using PBMCs with a small fraction (10%) of FL supports higher expansion compared to 100% labeled.

FIG. 31. Biotin 1 CAR expression-Long spacer. The method is compatible when using an anti-biotin CAR that harbors a long spacer. To explore the compatibility of using a CAR against biotin, normal PBMCs were labeled with 100% or 10% biotin and CD4 and CD8 cells were modified with a CAR targeting biotin. EGFRt, a marker of cell modification, was used to track cell purity after expansion. Cell counts were determined to assess overall cell expansion. A high purity of cells was achieved after expansion using nsPBMC addback that was either 100% or 10% labeled with biotin.

FIG. 32. Biotin 1 CAR expression-Short spacer. The method is compatible when using an anti-biotin CAR that harbors a short spacer. To explore the compatibility of using a CAR against biotin, normal PBMCs were labeled with 100% or 10% biotin and CD4 and CD8 cells were modified with a CAR targeting biotin. EGFRt, a marker of cell modification, was used to track cell purity after expansion. Cell counts were determined to assess overall cell expansion. A high purity of cells was achieved after expansion using PBMC addbacks that were either 100% or 10% labeled with biotin.

FIG. 33. A schematic representation of electroporation-based T Cell Interface for CAR-Limited Expansion (EP) with multiple pathways for cell-specific stimulation. Here, rDNA transposon payload and PiggyBac transposase mRNA are transiently delivered via electroporation. Cell-specific stimulation is achieved through addition of (1) autologous or allogenic PBMCs bearing target antigen (incl. CD19⁺ B cells); (2) autologous or allogenic PBMCs that have been preloaded with lipid-anchored hapten (reactive to an anti-hapten CAR; (3) autologous or allogenic PBMCs that have been incubated with ‘chemical adapters’ (e.g. antigen-Fc, anti-scFv, anti-Fab) each bispecific to both CAR and FcγRI or FcγRIIa expressed on monocytes to enable nsPBMC-to-CAR T cell crosslinking and subsequent activation; (4) T cells or autologous/allogenic nsPBMCs pre-transduced with CAR target; (5) virus-like particles (VLPs) that have been preloaded with CAR target.

FIG. 34. Non-transposable CD19CAR, transposable orthogonal CAR for generalized electroporation (EP) method. The CD19CAR has proven to be a robust CAR compatible with the expansion method. While expanding an orthogonal CAR is possible, here a vector format compatible with the process was conceived that allows for the expansion of any orthogonal CAR. Traditionally, CARs have always been housed within two flanking ITRs or the transposable element. To facilitate a CD19CAR stimulation (stim) which would allow for the expansion of an orthogonal CAR, the CD19CAR was moved outside of the ITR to avoid stable integration. This allows for transient expression of the CD19CAR which initiates expansion by providing transient T cell stimulation. Only cells that transpose the orthogonal CAR and accompanying DHFRdm cassette will survive in the presence of MTX selection.

FIG. 35. Self-inactivating PiggyBac or Sleeping Beauty Transposase Transposon vectors. Schematic representation of a self-inactivating piggyBac or Sleep Beauty Transposase system. Self-inactivating Transposase systems are compatible with the system allowing for simple modification of T-cells that are not reliant of sources of RNA to supply transposase protein.

FIG. 36. Self-inactivating & non-transposable CD19CAR PiggyBac or Sleeping Beauty Transposon for generalized electroporation method. A conception utilizing the self-inactivating transpoase system and a transient CD19CAR stim to expand an orthogonal CAR in either an all-in-one format or split across two constructs. Orthogonal CAR can use a self-inactivating transposase system that harbors a CD19CAR outside of the ITRs. The orthogonal CAR can include a non-PBMC scFv, hinge, 4-1BB, and CD3ζ. The CD19CAR can provide a transient stimulation to start the process and the transposase will self-inactivate once transposition is complete, transposing the orthogonal CAR cargo, resulting in the expansion of the orthogonal CAR.

FIG. 37. A schematic to diagram the viral-introduced process. The viral-introduced process is an unstimulated transduction that utilizes a small fraction of T cells competent for lentiviral uptake. It is expected that there is a portion of cells that undergo a pseudo-transduction event with subsequent transient expression of a CAR. Upon CAR engagement with its cognate antigen, stimulated T cells will spur along integration of vRNA and potential additional viral uptake resulting in high cell and vector positive yield.

FIG. 38. CD19CAR placed within the viral producer cells to make virus that packages orthogonal CAR cargo and expresses CD19CAR. Virus expressing CD19CAR but carrying orthogonal CAR cargo could infect T cells, provide a transient stimulation and spur the integration and high yield and purity. This conception is another way to provide a robust but transient CD19CAR stimulation to drive the expansion of orthogonal cargo.

FIG. 39. Sequences supporting the disclosure including: pJ04052—From EF1a to RGB\polyA (SEQ ID NO: 1), pJ04457—from S1-61 to BGH poly A (SEQ ID NO: 2), pJ04831 MNDp_GFP: Ffluc_RBGPolyA_2x(3′HSA)_EF1a(I)_huCD19scFv(G01S)-IgG4hinge-CD28tm-41BB-zeta-P2A-DHFRdm-T2A-EGFRt_BGHPolyA_PBNP (SEQ ID NO: 3), pJ04788, S1-61-IL2mp_PD1(A99L): MYD88-T2A-Her2tG_EF1a(I)_huCD19scFv(G01S)-IgG4hinge-CD28tm-41BB-zeta-P2A-DHFRdm-T2A-EGFRt_PBNP (SEQ ID NO: 4), P2A (SEQ ID NO: 5), T2A (SEQ ID NO: 6), E2A (SEQ ID NO: 7), F2A (SEQ ID NO: 8), 4-1BB (SEQ ID NO: 9), CD3ζ (SEQ ID NO: 10), CD28_Transmembrane domain (SEQ ID NO: 11), GFP: Ffluc (SEQ ID NO: 12), GFP (SEQ ID NO: 13), firefly luciferase reporter vector pGL4.27 [luc2P/minP/Hygro]; GenBank: ABI94448.1 (SEQ ID NO: 14), huCD19scFv(G01S) (SEQ ID NO: 15), IgG4 hinge (SEQ ID NO: 16), CD3ζ (SEQ ID NO: 17), 4-1BB (SEQ ID NO: 18), Her2tG (SEQ ID NO: 19), EGFRt (SEQ ID NO: 20), DHFRdm (SEQ ID NO: 21), PD1TM-MyD88 (SEQ ID NO: 22), EF1a full length promoter (SEQ ID NO: 23), EF1a(L) promoter (SEQ ID NO: 67), EF1a(s) promoter (SEQ ID NO: 68), MND promoter (SEQ ID NO: 24), S1-61 (iSynPro) (SEQ ID NO: 25), IL2 minimal promoter (SEQ ID NO: 26), BGH poly A (SEQ ID NO: 27), RBGPolyA (SEQ ID NO: 28), huCD19 (G01S) scFv (SEQ ID NO: 29), muCD19 (FMC63) scFv (SEQ ID NO: 30), CD20 (Leu 16) scFv (SEQ ID NO: 31), CD22 (m971) scFv (SEQ ID NO: 32), B7H3 (hBRCA84D) scFv (SEQ ID NO: 33), L1CAM (CE7) scFv (SEQ ID NO: 34), EGFR scFv (SEQ ID NO: 35), EGFRVIII (806) scFv (SEQ ID NO: 36), EphA2 (2A4) scFv (SEQ ID NO: 37), EpHA2 (4H5) scFv (SEQ ID NO: 38), FITC (E2) scFv (SEQ ID NO: 39), GD2 (hu3F8) scFv (SEQ ID NO: 40), Her2 (Herceptin) scFv (SEQ ID NO: 41), IL13Ra2 (hu08) VIVh scFv (SEQ ID NO: 42), IL13Ra2 hu08 VhVI scFv (SEQ ID NO: 43), IL13Ra2 (hu07) VhVI scFv (SEQ ID NO: 44), IL13Ra2 (hu07) VhVI scFv (SEQ ID NO: 45), oaGD2 (8B6) VIVh scFv (SEQ ID NO: 46), ROR1 (R12) scFv (SEQ ID NO: 47), CD33 (h2H12) VhVI scFv (SEQ ID NO: 48), CD33 (h2H12) VIVh scFv (SEQ ID NO: 49), Mesothelin (P4) scFv (SEQ ID NO: 50), VAR2CSA (ID1-DBL2Xb) scFv (SEQ ID NO: 51), and Anti-IL13Ra2 (IL13 zetakine) amino acid sequence (SEQ ID NO: 52).

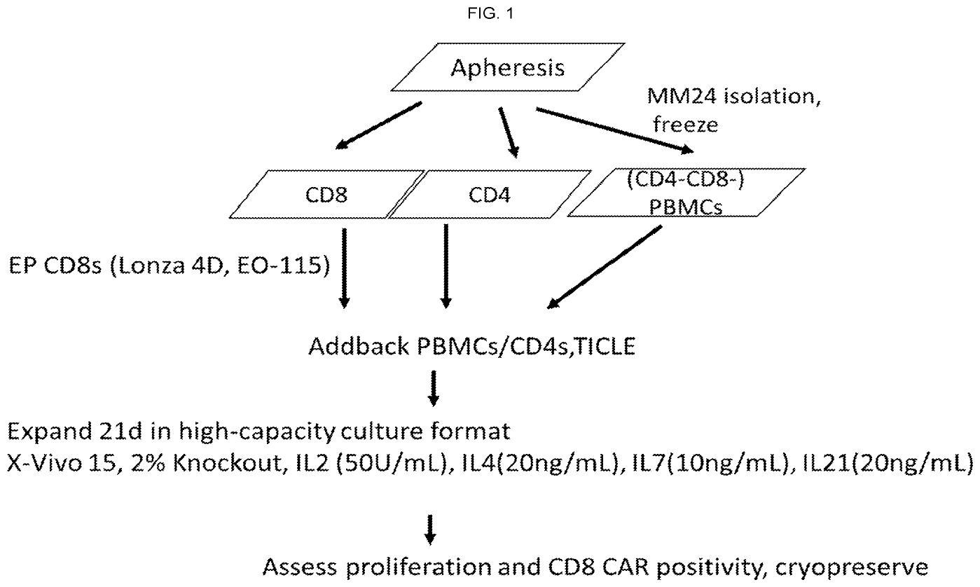

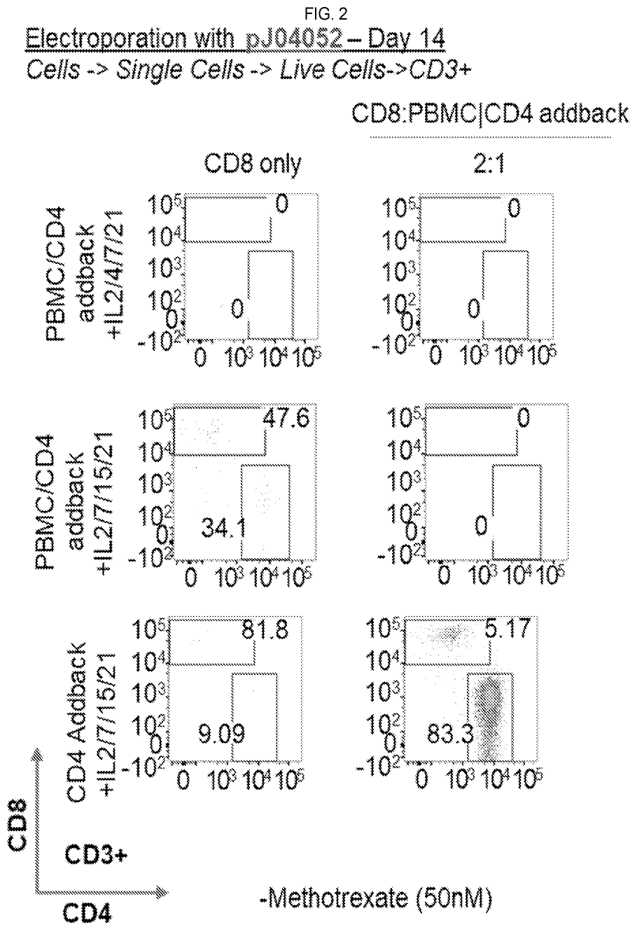

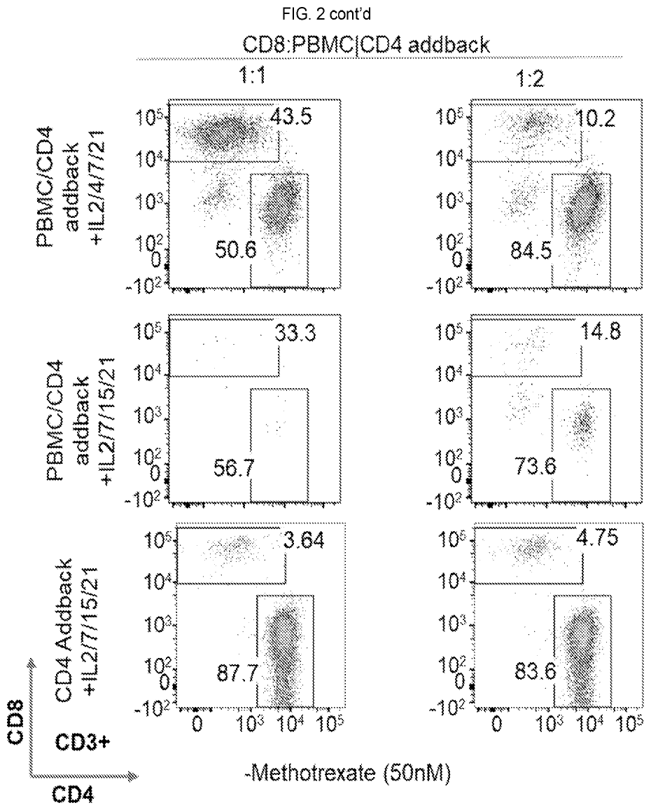

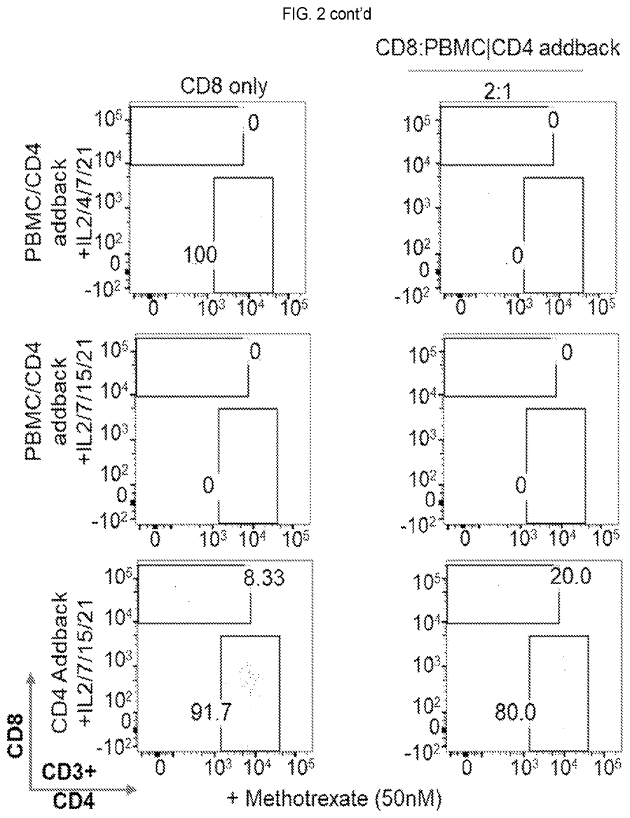

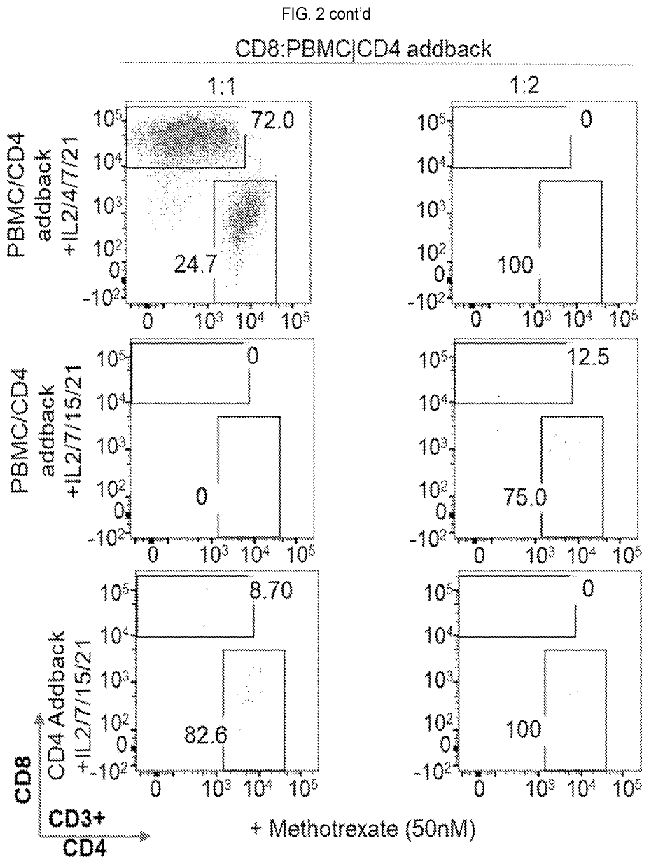

### DETAILED DESCRIPTION

A variety of therapies developed over the last few decades engineer T cells to express a recombinant receptor that binds antigens on unwanted cell types (e.g., cancer cells, virally infected cells). When the recombinant receptor binds the target antigen, the T cell can be activated to destroy the bound cell type. Chimeric antigen receptor (CAR)-T cell therapy is one such therapy.

Chimeric antigen receptor (CAR)-T cell therapy involves the engineering of T cells to express synthetic receptors designed to target cells such as cancer cells or virally infected cells. Currently, the standard manufacture of CAR-T cells may not generate sufficient numbers of CAR-expressing T cells to provide therapeutic benefit, particularly when the starting number of cells is small at the beginning of a manufacturing process. Currently used procedures also use lentiviral or gamma-retroviral transduction of T cells. Although this method has shown remarkable efficacy in clinical trials, the potential for insertional mutagenesis and genotoxicity of viral vectors causes concern. Furthermore, viral transduction is limited by the size of deliverable DNA and larger constructs have reduced transfection efficiency. As such, there is a need for a method of CAR-T manufacture that results in clinical-grade CAR-T cells with high CAR positivity and fold expansion that is amenable to large, complex vector constructs. Such advances would expand the clinical translation of this important therapeutic medicine.

The current disclosure provides methods to produce a population of recombinant receptor-expressing T cells with high cell count and recombinant receptor positivity from limited starting cell counts. In certain examples, the current disclosure provides T cell manufacturing methods that utilize autologous cell types to stimulate T cells during manufacture. In certain examples, the T cells express a binding domain that binds an immune cell activating epitope on the stimulating autologous cell types and/or a multi-specific binding molecule (e.g., a bi-specific antibody).

In certain examples, the T cells express a recombinant receptor (e.g., a CAR) that includes a binding domain that binds an immune cell activating epitope expressed by the stimulating autologous cell types and/or a multi-specific binding molecule (e.g., a bi-specific antibody). The immune cell activating epitope can be naturally expressed by the stimulating autologous cell type or the stimulating autologous cell type can be genetically modified to express the immune cell activating epitope. In each of these approaches, the methods rely on more natural cell activation signals such as costimulatory molecules and avoid the need to rely on commonly used artificial stimulation reagents, such as CD3 and/or CD28 stimulating reagents (e.g., Dynabeads® (Invitrogen, Waltham, MA) or T Cell TransAct™, (Miltenyi, Bergisch Gladbach, Germany)).

In certain examples, the immune cell activating epitope is a B cell ligand. Exemplary B cell ligands include CD19, CD20, CD34, CD38, and/or CD45R. In other examples, the immune cell activating epitope is present on the myeloid cell population, such as CD33.

In particular embodiments, the recombinant receptor-expressing T cells are CD8+ and/or CD4+ T cells.

In particular embodiments, the methods include obtaining T cells positive for a marker from a sample, retaining the negative fraction of the sample, modifying obtained T cells to express a recombinant receptor, and culturing the recombinant receptor-expressing T cells within the negative fraction, wherein a binding domain expressed by the recombinant receptor-expressing T cells binds an immune cell activating epitope. In particular embodiments, the recombinant receptor binds the immune cell activating epitope. In particular embodiments, the immune cell activating epitope is expressed by an immune cell within the negative fraction and/or is a binding domain on a multi-specific binding molecule.

In particular embodiments, examples of these methods include:
obtaining CD8+ cells isolated from a sample;
obtaining CD4+ cells isolated from the same sample;
retaining the sample following the CD8+ cell isolation and the CD4+ cell isolation (the negative fraction);
introducing a chimeric antigen receptor (CAR)-encoding genetic construct into the isolated CD8+ T cells, wherein the CAR-encoding genetic construct results in expression of a CAR by the CD8+ T cells, and wherein the CD8+ T cells express a binding domain that binds an immune cell activating epitope; and
co-culturing the CAR-expressing CD8+ cells with the negative fraction within a cytokine-supplemented media.

In particular embodiments, the co-culturing includes co-culturing the CAR-expressing CD8+ cells with the isolated CD4+ cells and the negative fraction within a cytokine-supplemented media.

In other embodiments, the methods include:
obtaining CD8+ cells isolated from a sample;
obtaining CD4+ cells isolated from the same sample;
retaining the sample following the CD8+ cell isolation and the CD4+ cell isolation (the negative fraction);
introducing a CAR-encoding genetic construct into the isolated CD4+ T cells, wherein the CAR-encoding genetic construct results in expression of a CAR by the CD4+ T cells, and wherein the CD4+ T cells express a binding domain that binds an immune cell activating epitope; and
co-culturing the CAR-expressing CD4+ cells with the negative fraction within a cytokine-supplemented media.

In particular embodiments, the co-culturing includes co-culturing the CAR-expressing CD4+ cells with the isolated CD8+ cells and the negative fraction within a cytokine-supplemented media.

In other embodiments, the methods include:
obtaining CD8+ cells isolated from a sample;
retaining the sample following the CD8+ cell isolation (the negative fraction);
introducing a CAR-encoding genetic construct into the isolated CD8+ T cells, wherein the CAR-encoding genetic construct results in expression of a CAR by the CD8+ T cells, and wherein the CD8+ T cells express a binding domain that binds an immune cell activating epitope; and
co-culturing the CAR-expressing CD8+ cells with the negative fraction within a cytokine-supplemented media.

In other embodiments, the methods include:
obtaining CD4+ cells isolated from a sample;
retaining the sample following the CD4+ cell isolation (the negative fraction);
introducing a CAR-encoding genetic construct into the isolated CD4+ T cells, wherein the CAR-encoding genetic construct results in expression of a CAR by the CD4+ T cells, and wherein the CD4+ T cells express a binding domain that binds an immune cell activating epitope; and
co-culturing the CAR-expressing CD4+ cells with the negative fraction within a cytokine-supplemented media.

In particular embodiments, the immune cell activating epitope is a portion of a molecule expressed by an immune cell within the negative fraction and/or a binding domain on a multi-specific binding molecule (e.g., bi-specific antibody). The binding domain that binds the immune cell activating epitope can be the binding domain of the CAR or recombinant receptor.

The early separation of T cells (e.g., CD8+ or CD4+ cells) from stimulating autologous cell types (e.g., negative fraction) before introducing a recombinant receptor-encoding genetic construct into the isolated T cells provides the benefit of modification of desired cell types. The introduction of a recombinant receptor-encoding genetic construct into unwanted cell types could have dangerous effects in vivo. Another benefit of the methods described herein is that the fraction of stimulating autologous cell types (i.e., negative fraction) remain in the cell population long enough to provide stimulation but yield to the T cell population after 3-5 days.

In certain examples, the ratio of CD8+ T cells to negative fraction during a co-culture is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 3:2, or 3:4. In certain examples, the ratio of CD8+ T cells to CD4+ T cells to negative fraction during a co-culture is 1:1:1, 1:1:2, 1:1:3 1:1:4, 1:1:5, 1:1:6, 1:1:7, 1:1:8, 1:1:9, 1:1:10, 1:1:11, 1:1:12, 1:1:13, 1:1:14, 1:1:15, 1:1:16, 1:1:17, 1:1:18, 1:1:19, 1:1:20, 2:1:1, 3:1:2, or 3:2:4. Ratios of 1:1:2, 1:1:3 1:1:4, 1:1:5, 1:1:6 are beneficial because they preserve more of the negative fraction for additional rounds of addback and co-culture.

In certain examples, cytokines within the cytokine-supplemented media include interleukin (IL)-4. Additional examples include media supplemented with IL4, IL7, and IL21. In certain examples, these cytokines can be present in concentrations of, for example, 5-40 ng/ml IL4, 5-20 ng/ml IL7, and 1-40 ng/ml IL21. In more particular examples, these cytokines can be present in concentrations of, for example, 20 ng/ml IL4, 10 ng/ml IL7, and 20 ng/mL IL21. Additional examples include IL-2, IL4, IL7, and IL21. In certain examples, these cytokines can be present in concentrations of, for example, 5-150 U/mL IL2, 5-40 ng/ml IL4, 5-20 ng/ml IL7, and 1-40 ng/mL IL21. In more particular examples, these cytokines can be present in concentrations of, for example, 50 U/mL IL2, 20 ng/ml IL4, 10 ng/ml IL7, and 20 ng/ml IL21. The media can be a serum-free media supplemented with serum replacement.

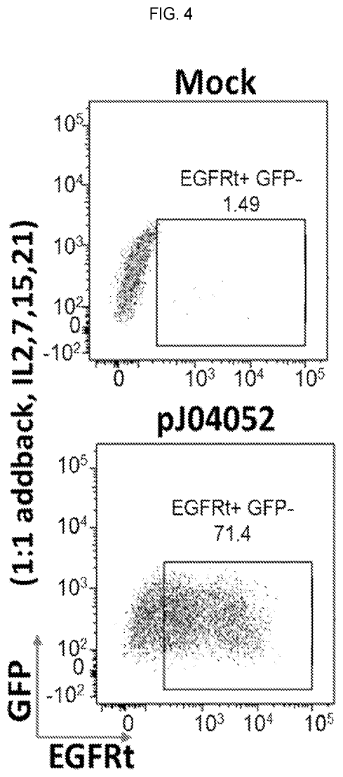

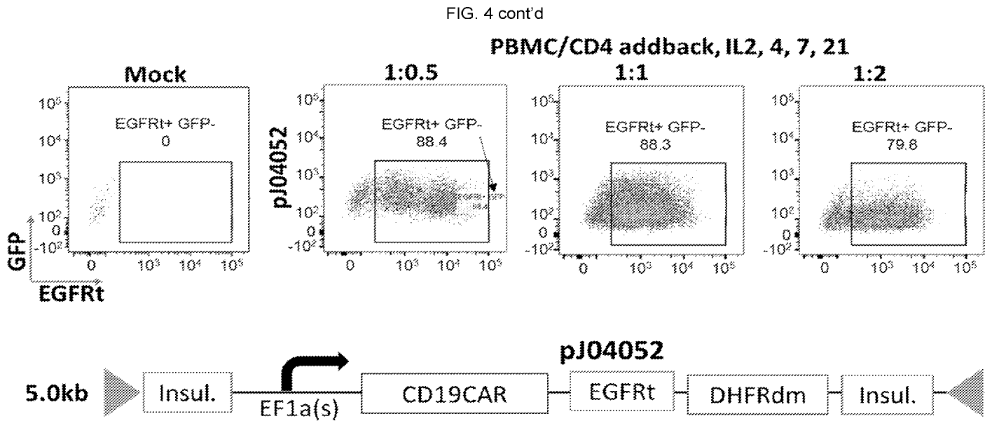

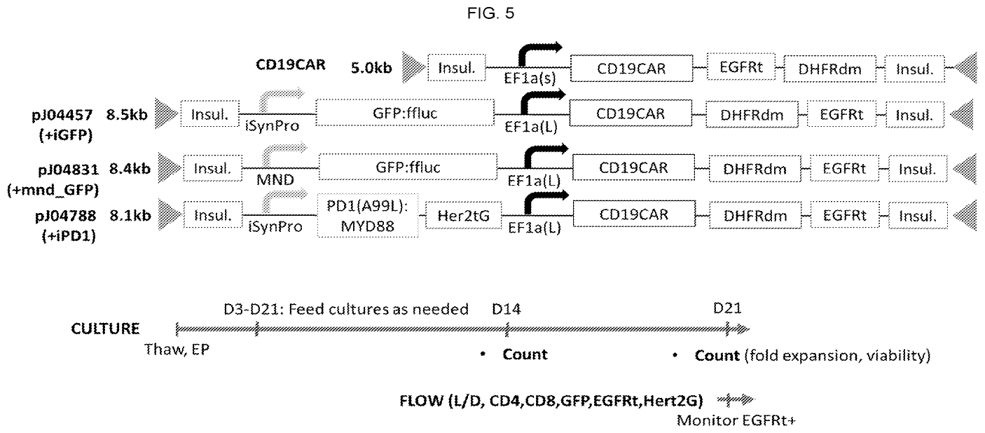

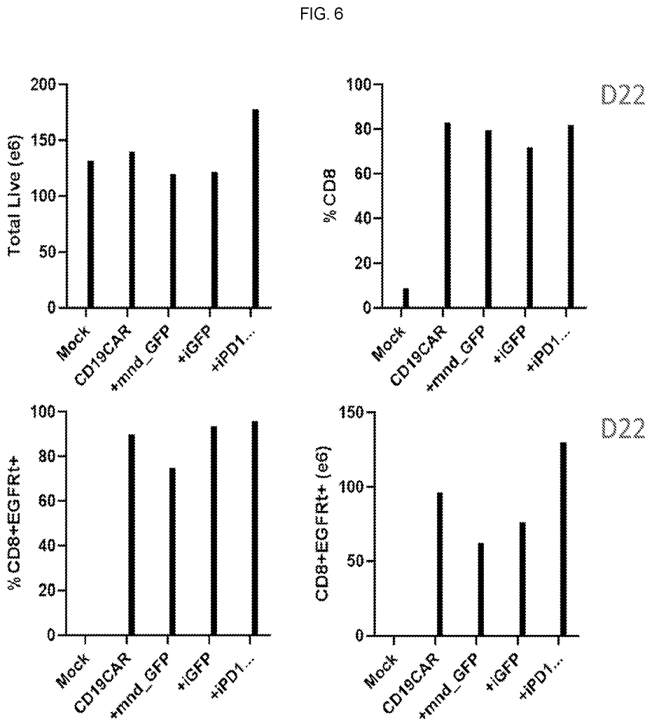

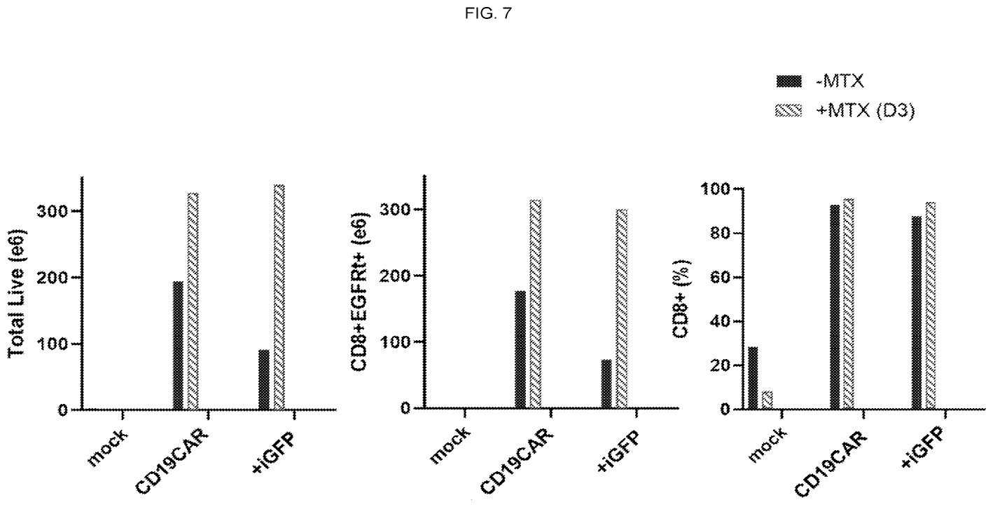

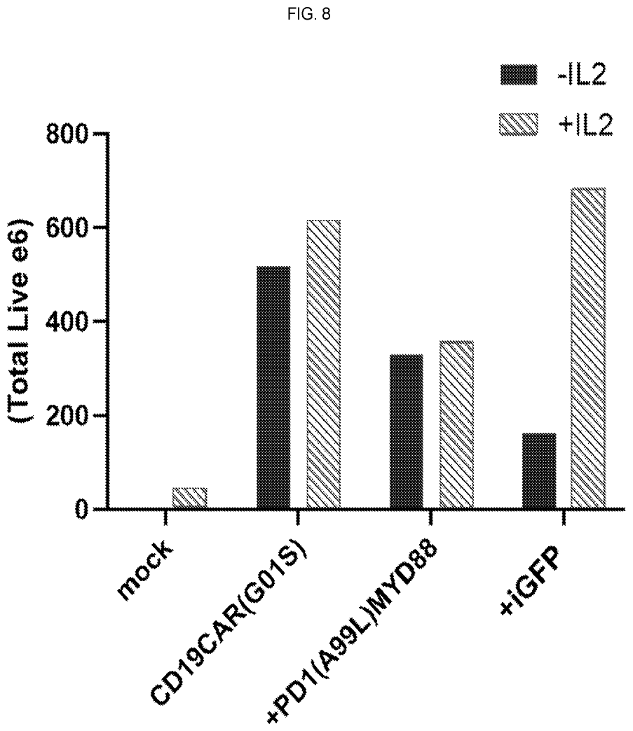

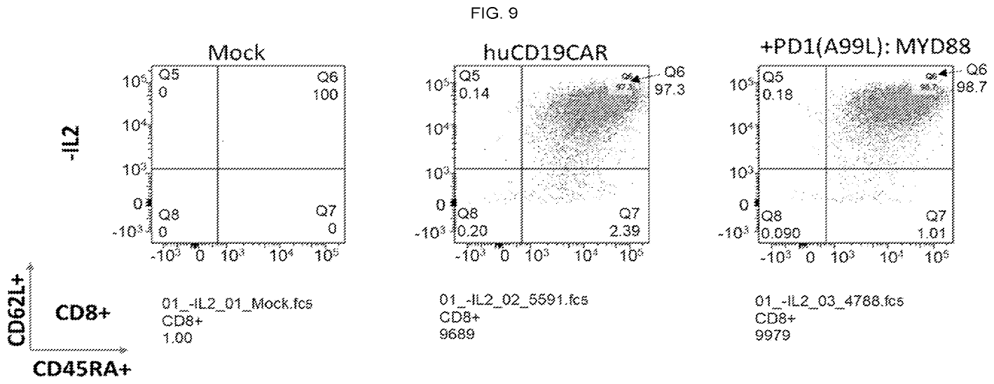

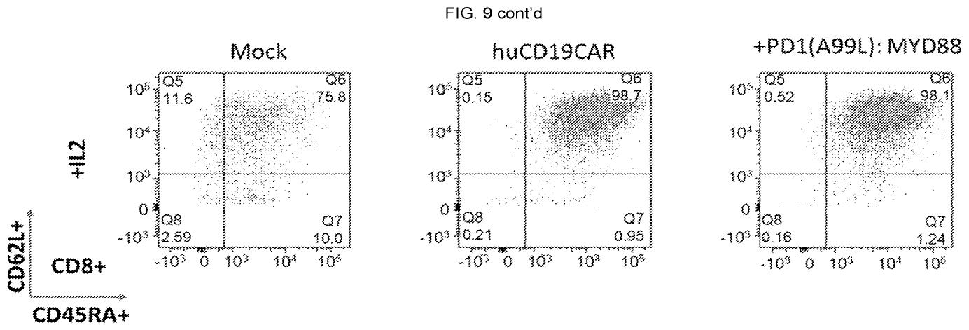

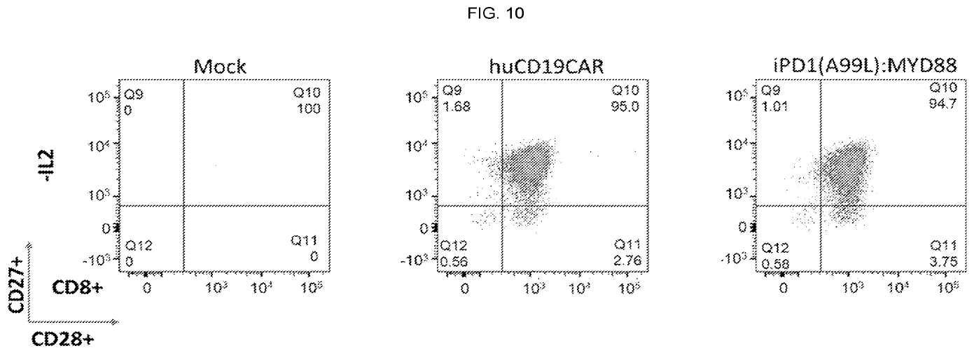

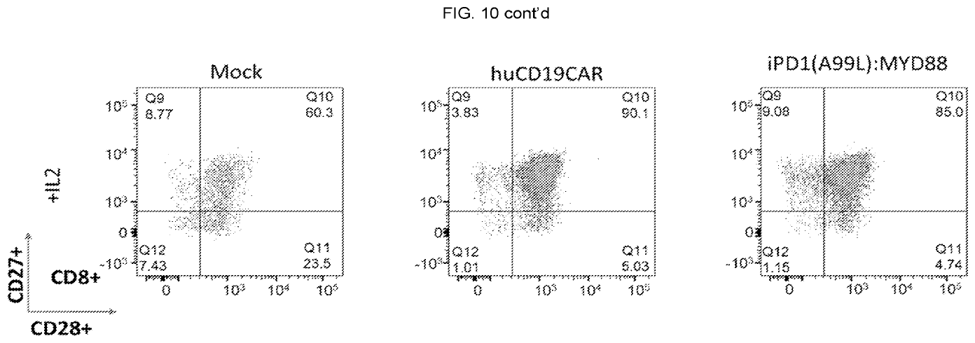

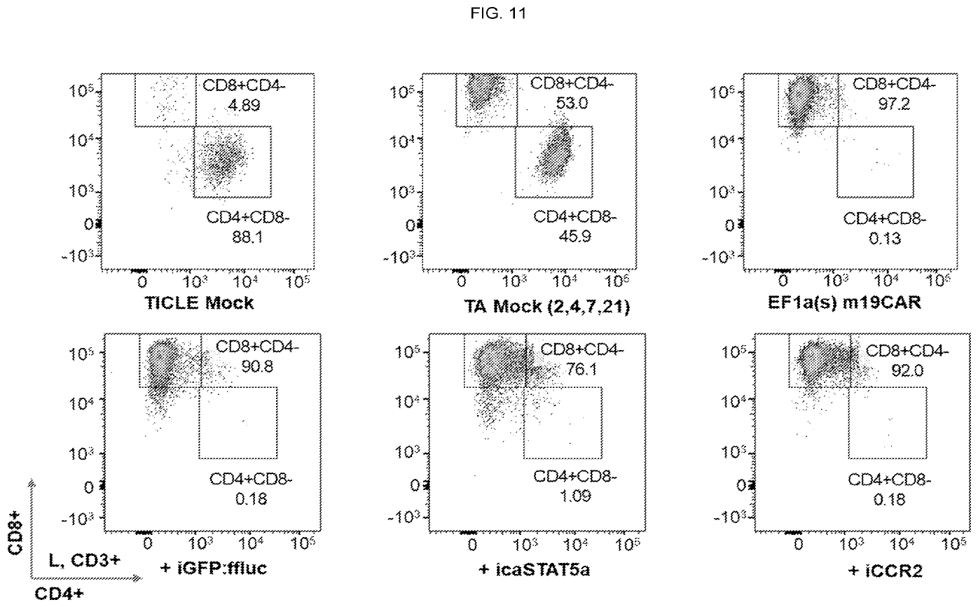

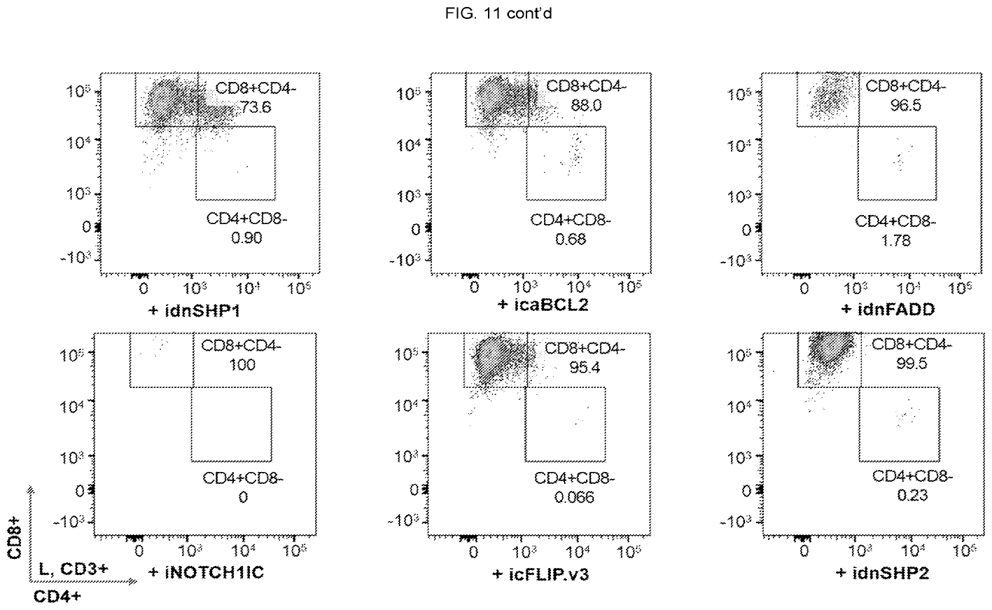

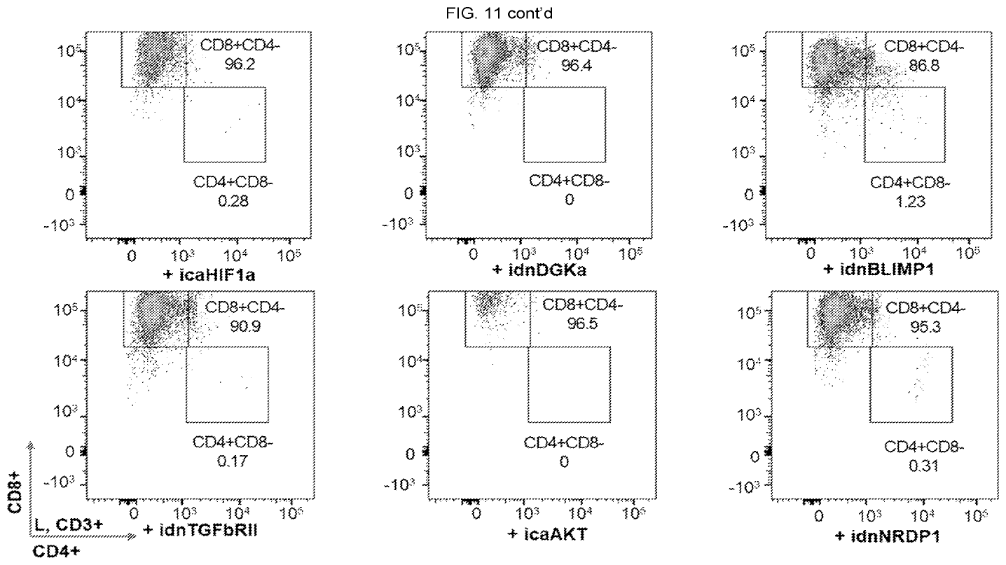

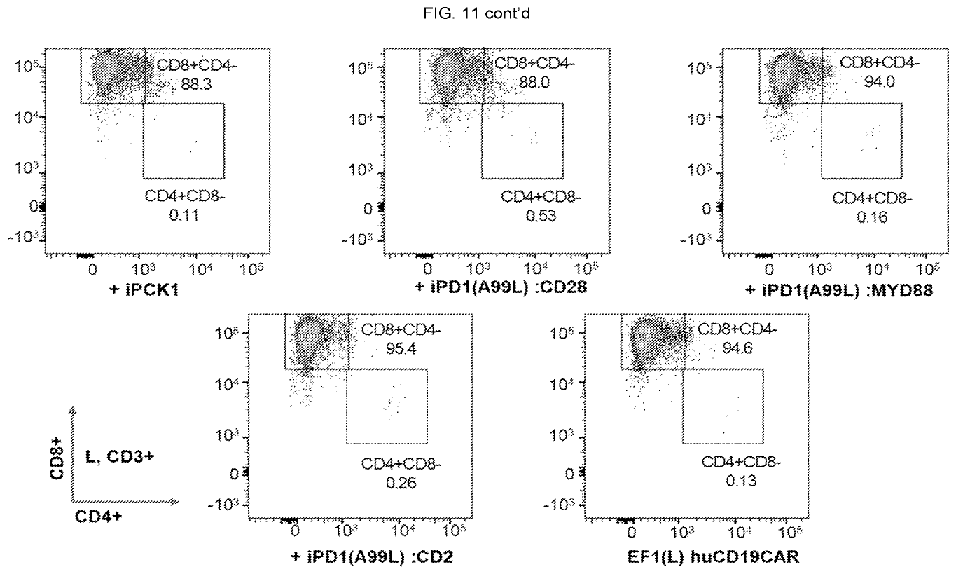

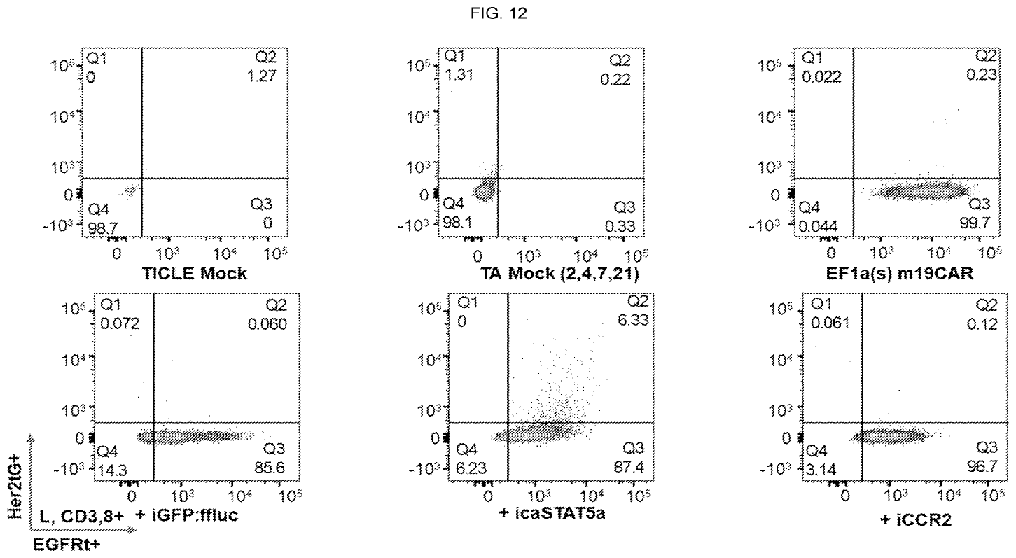

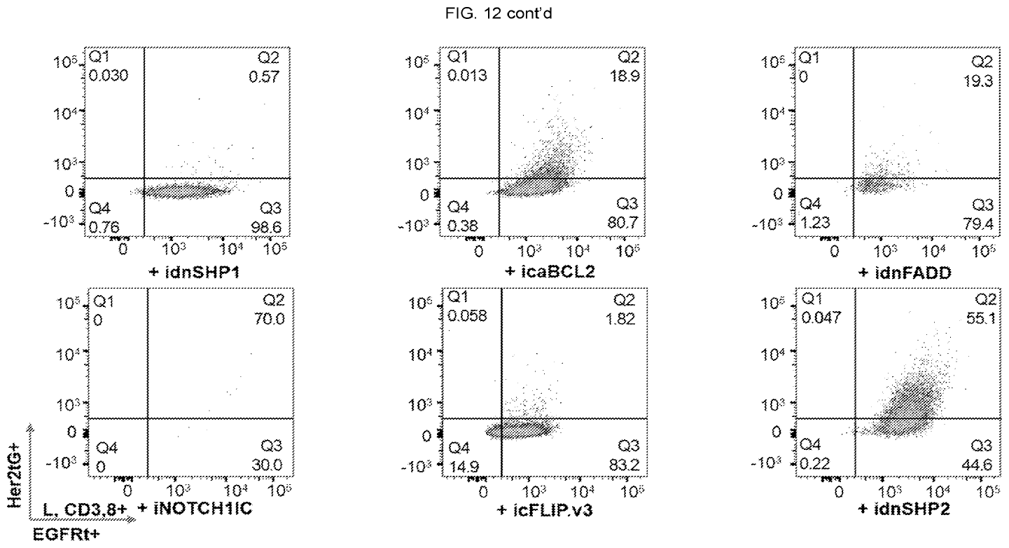

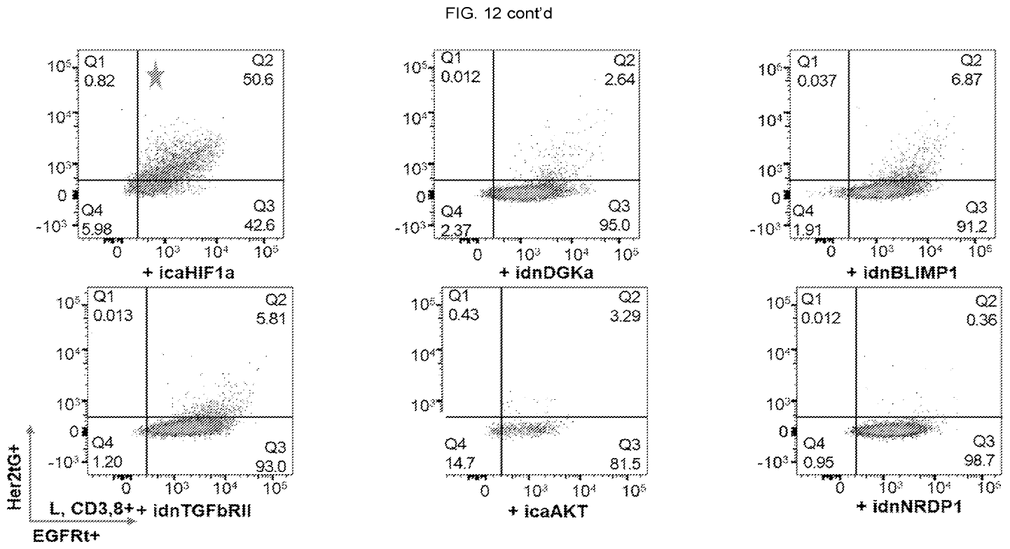

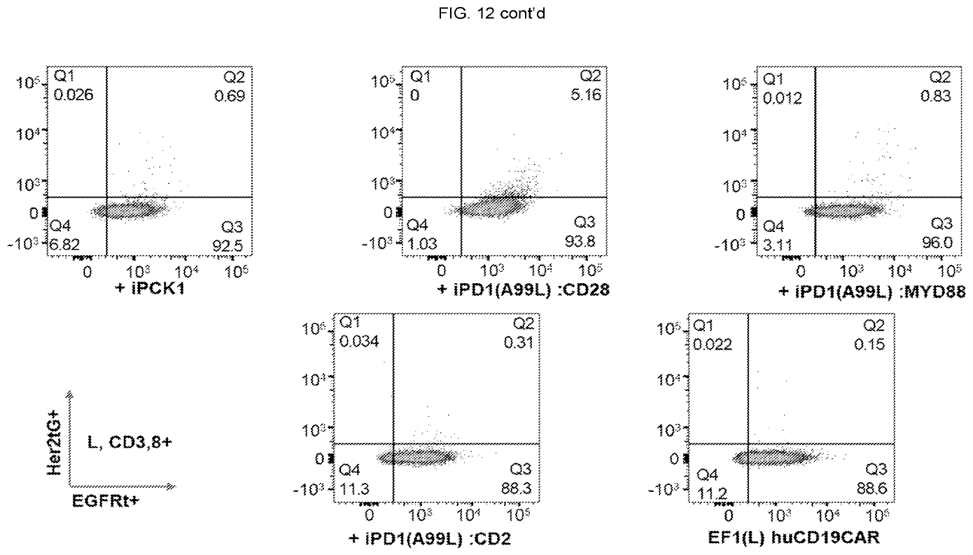

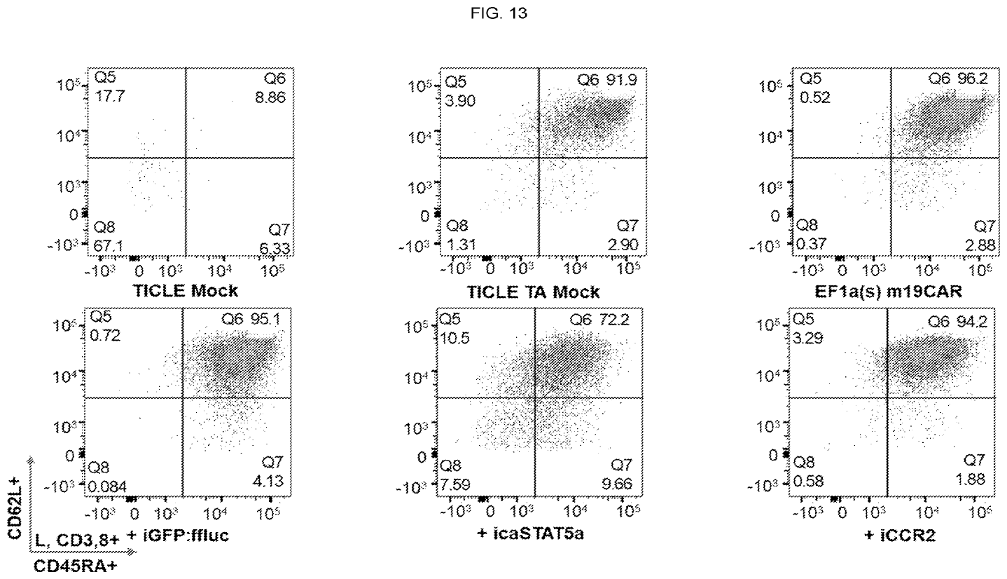

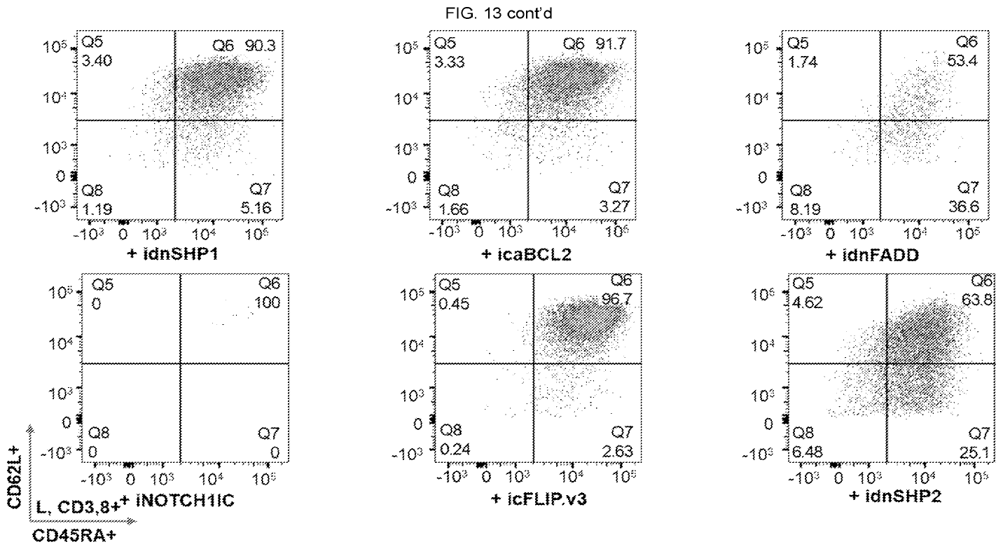

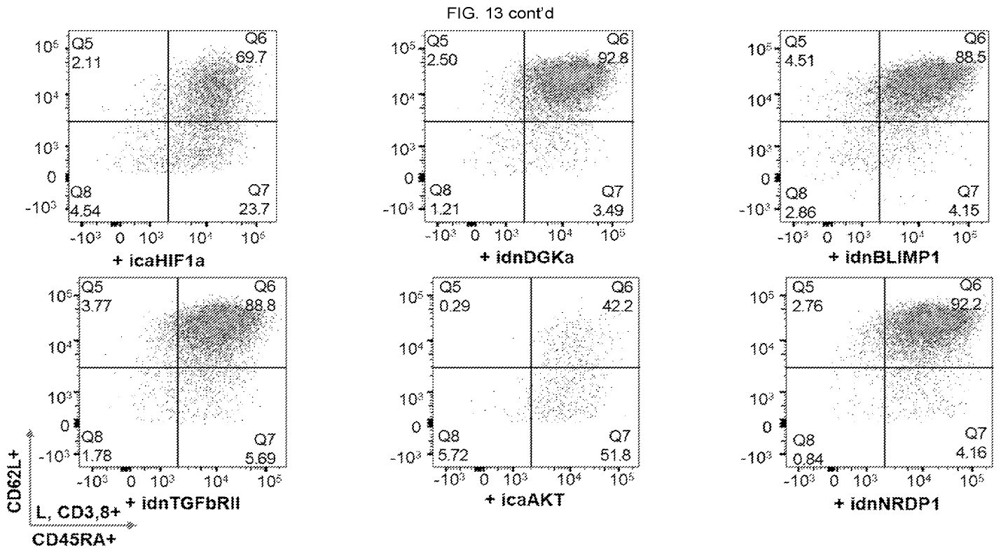

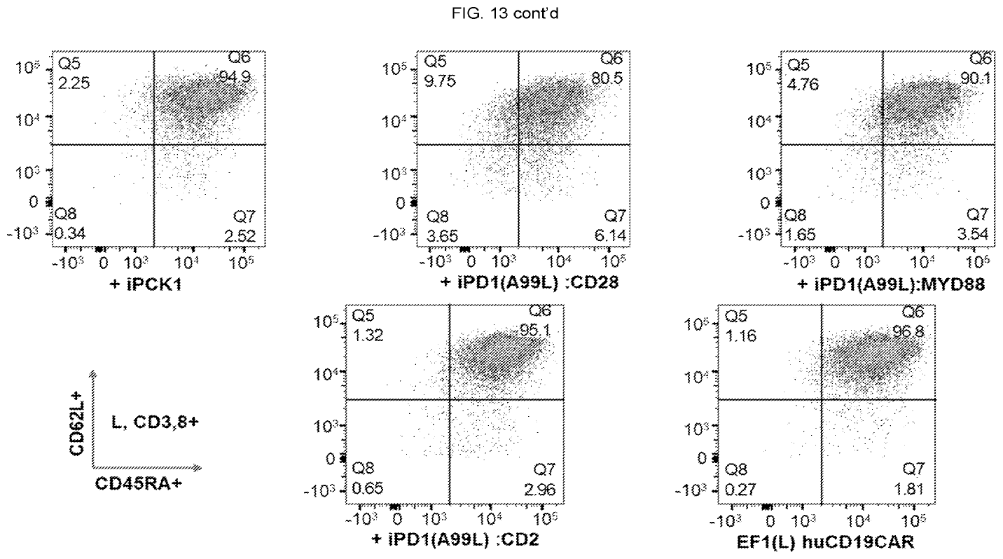

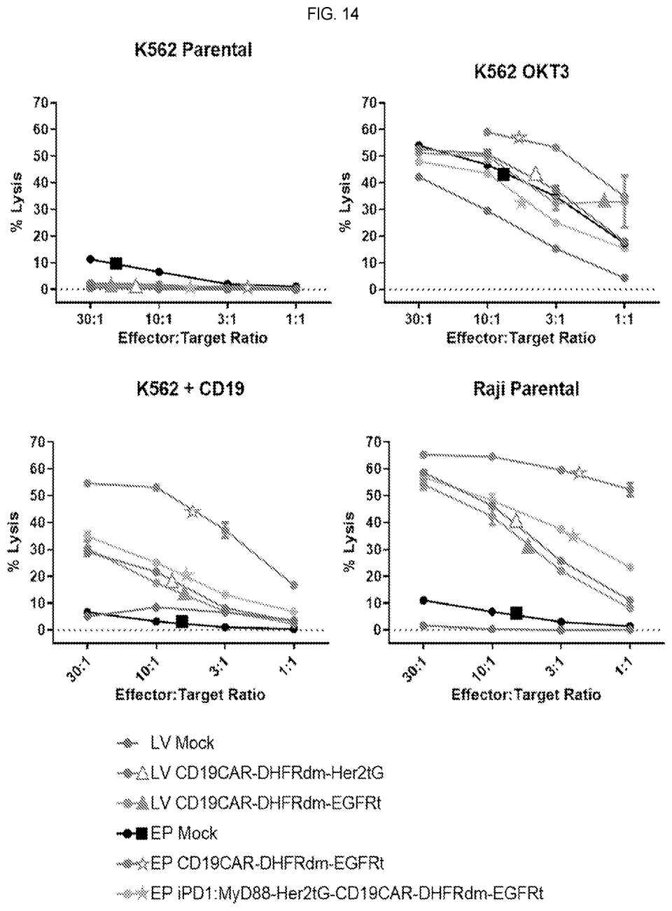

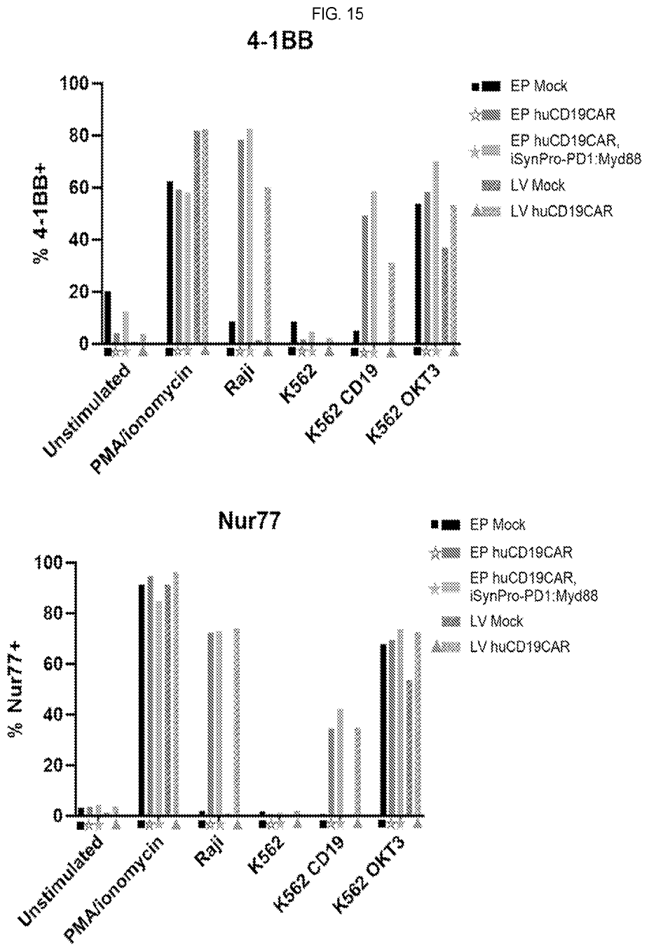

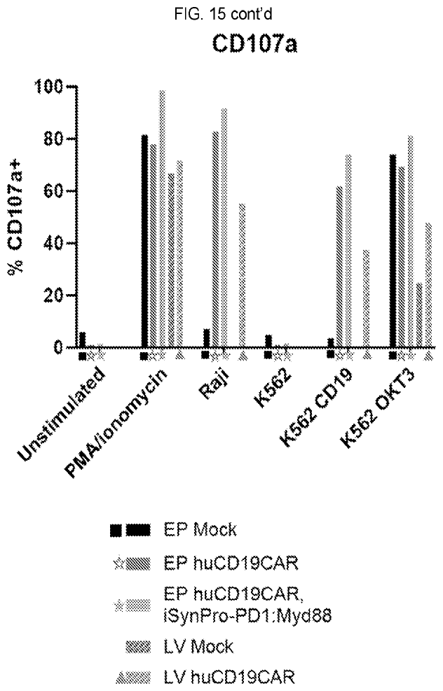

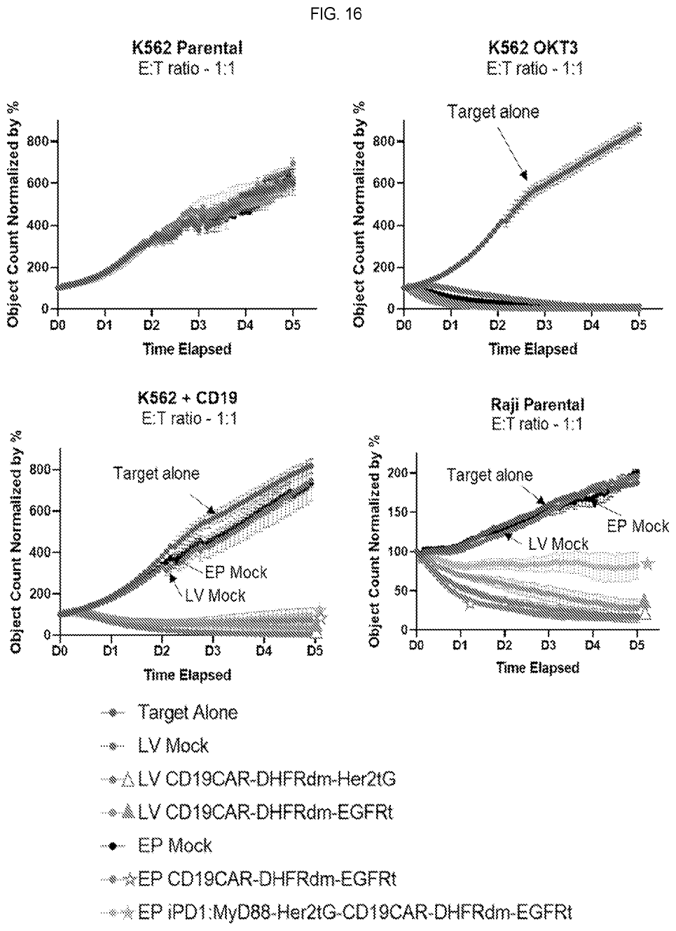

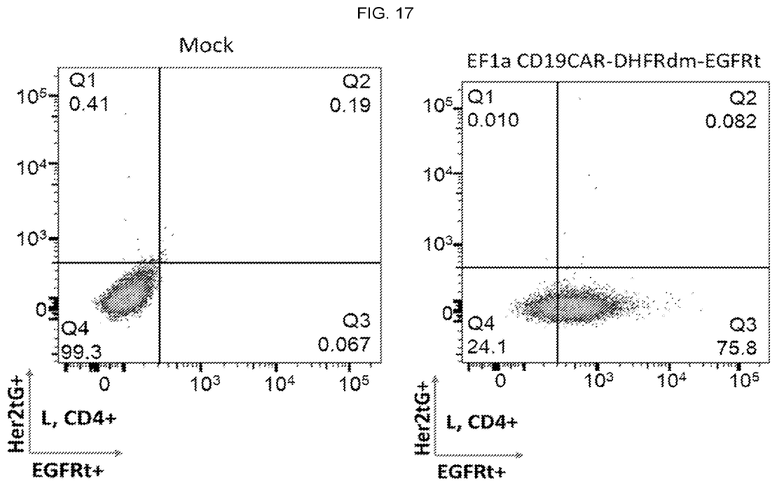

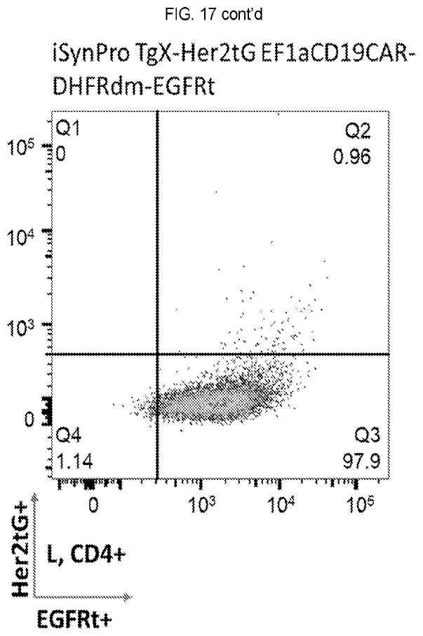

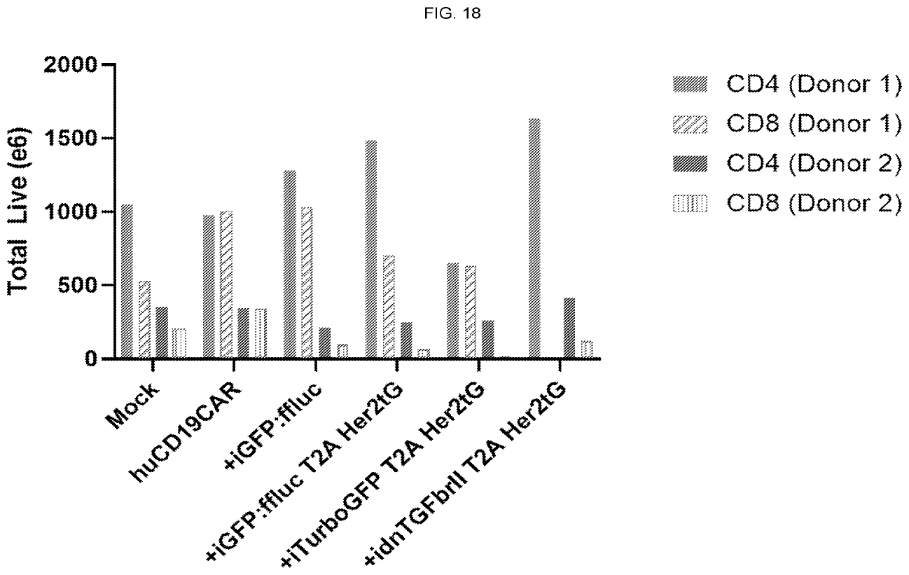

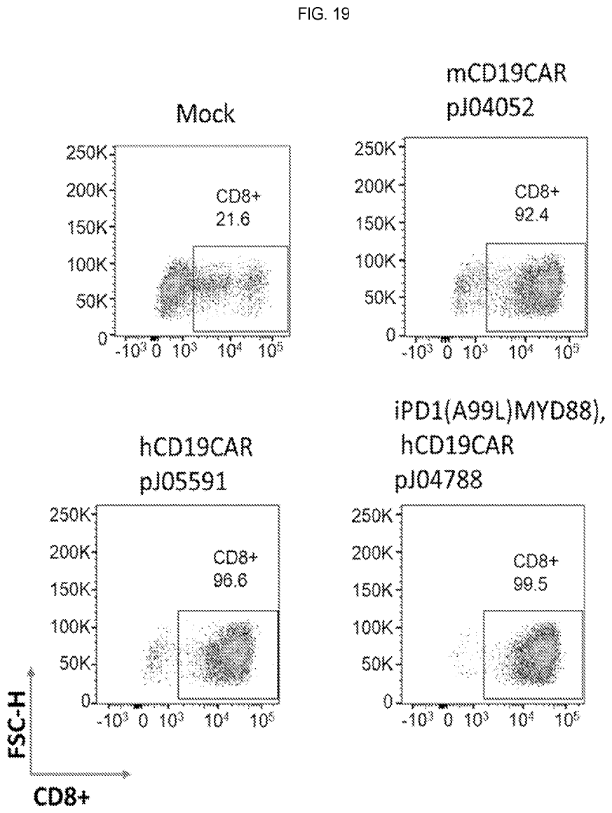

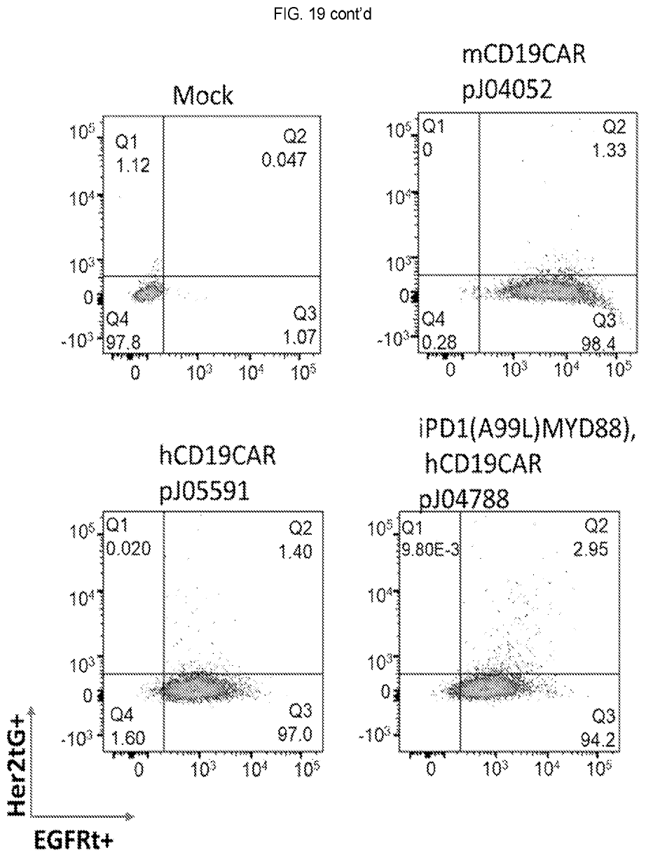

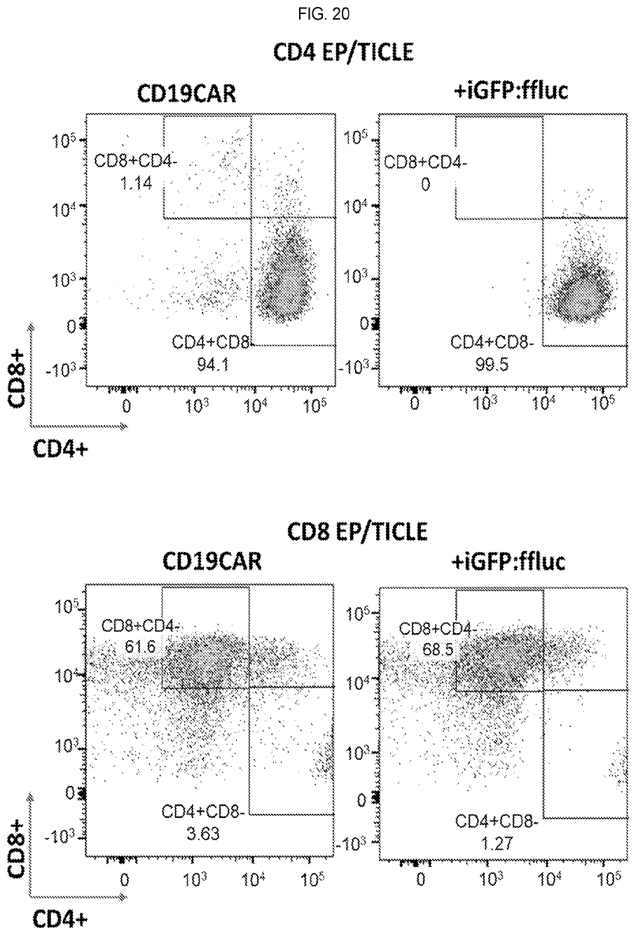

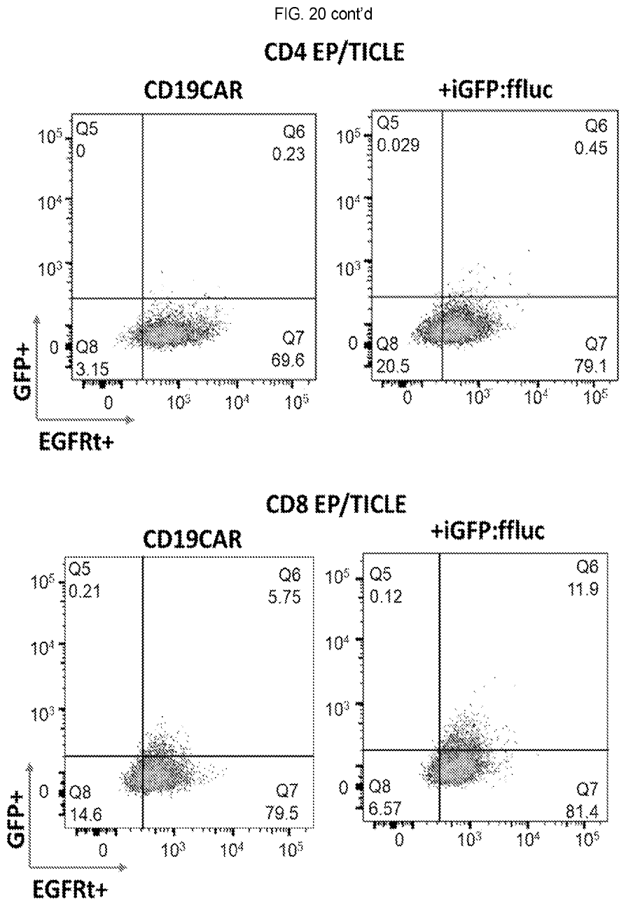

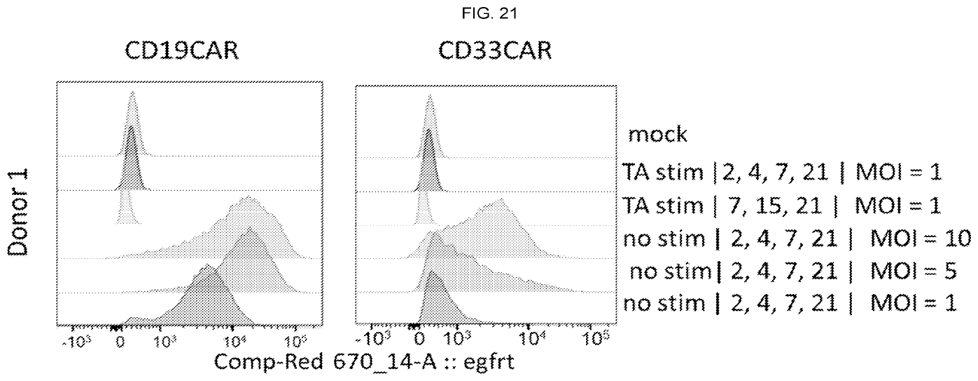

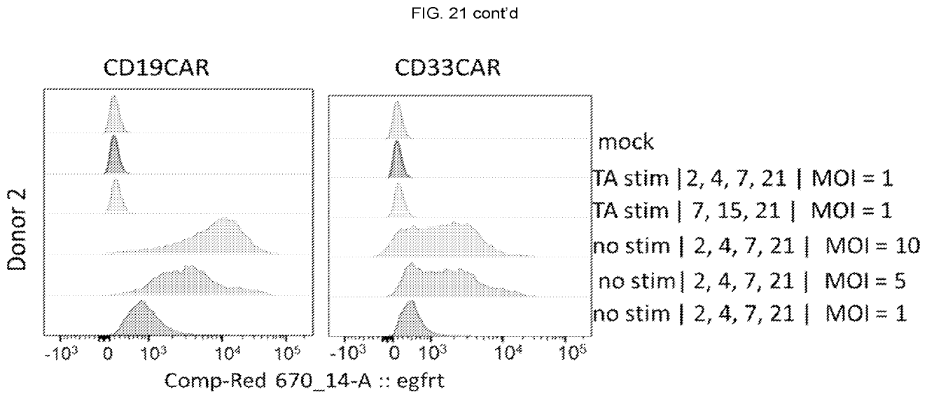

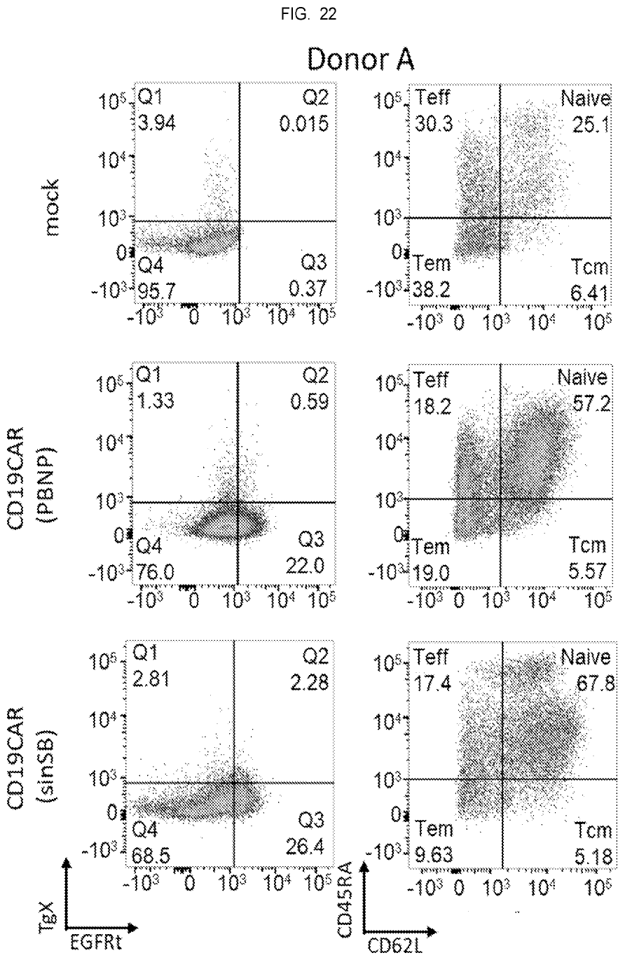

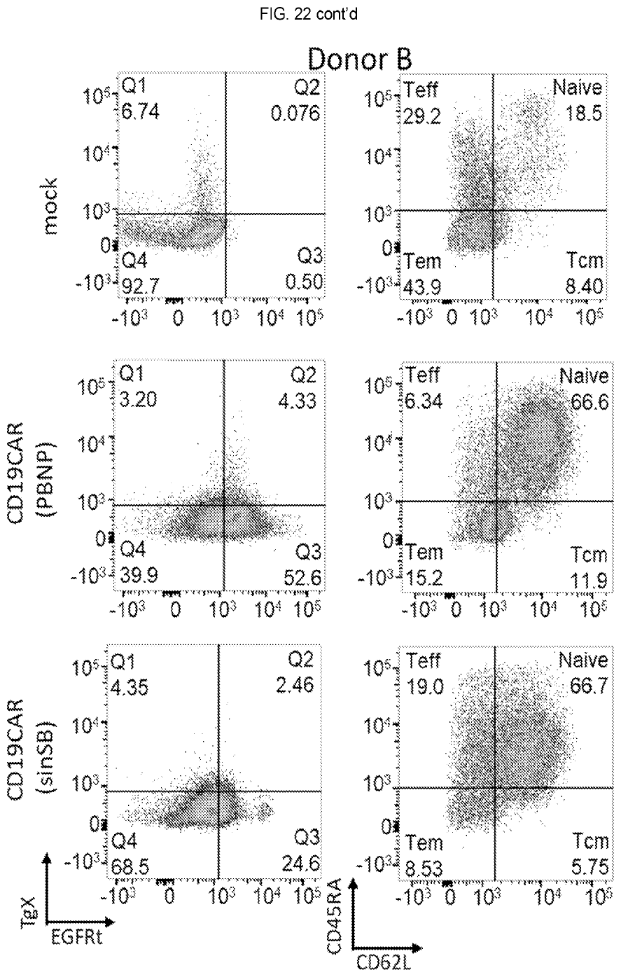

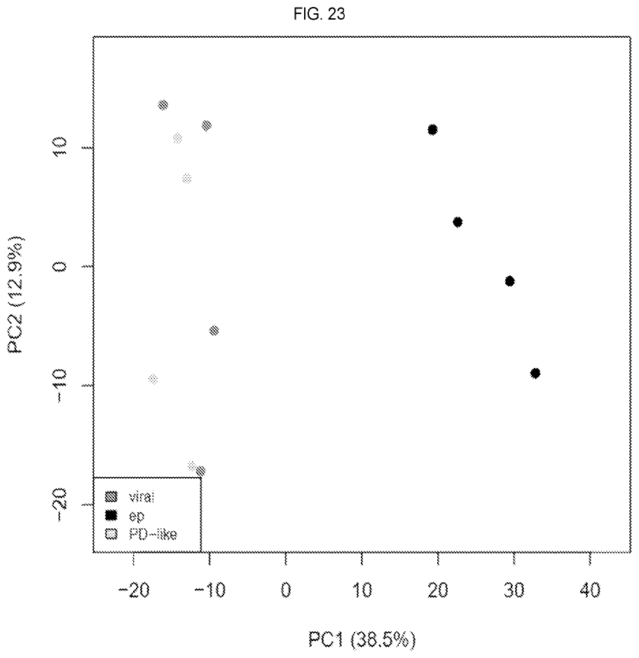

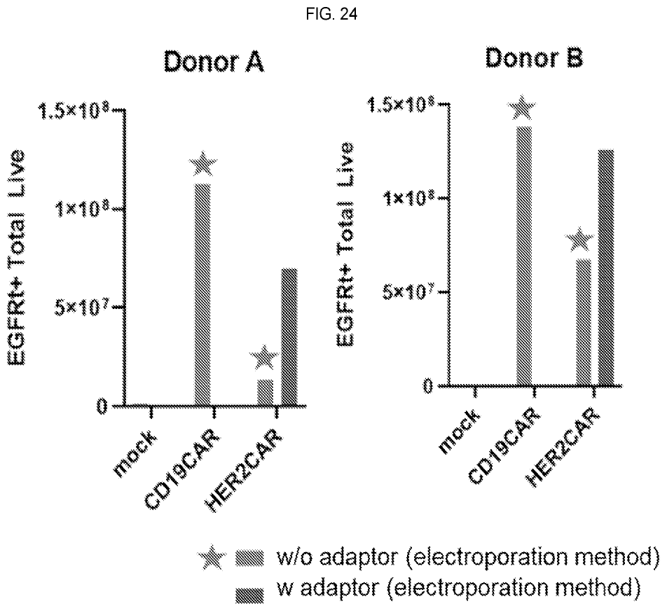

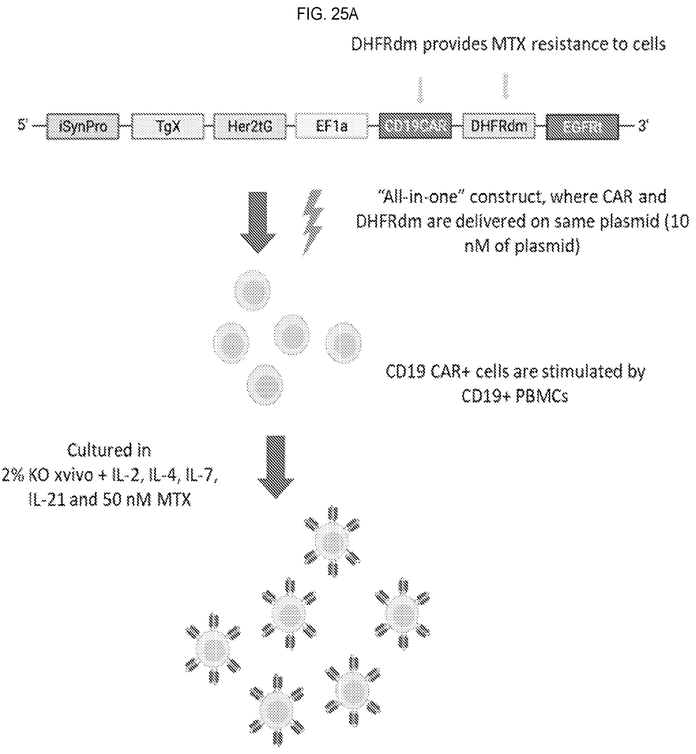

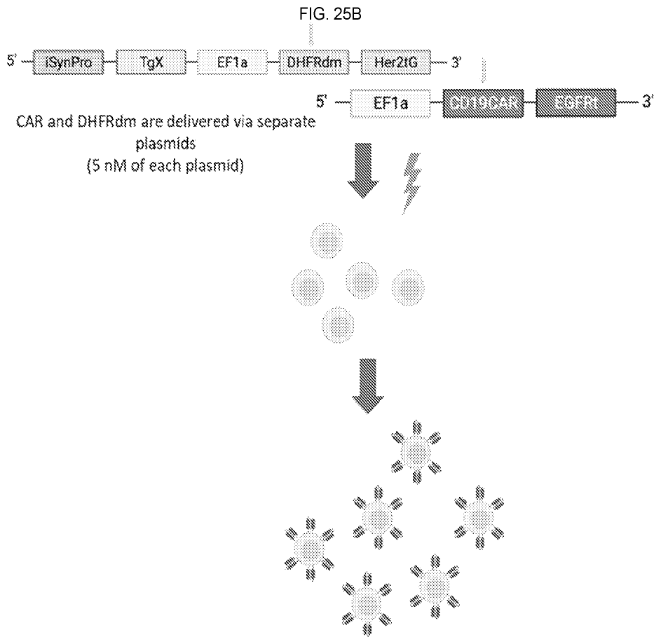

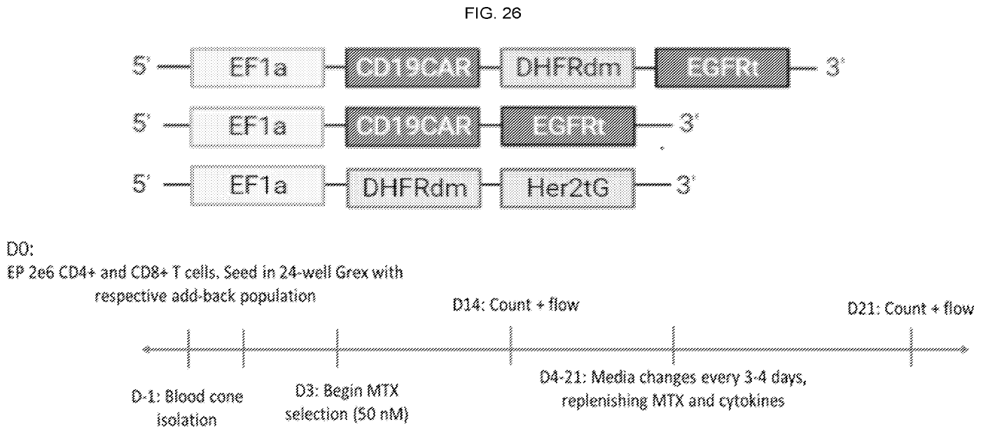

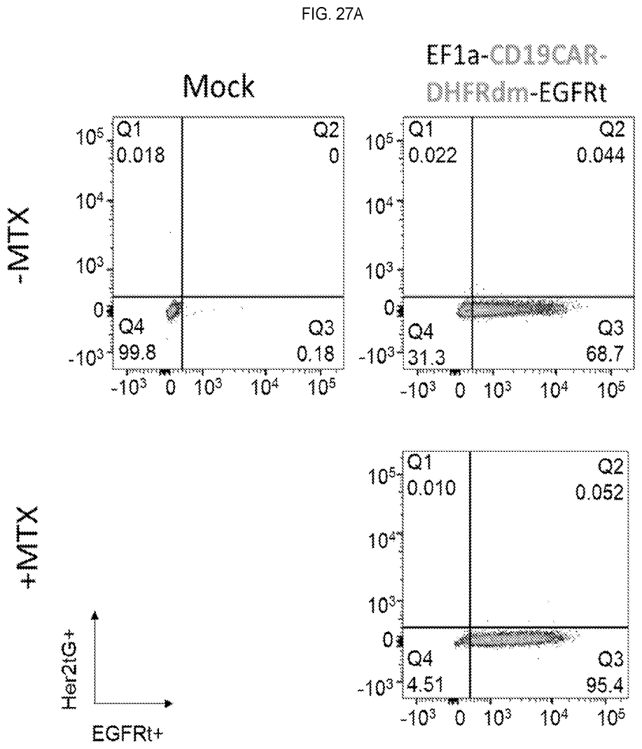

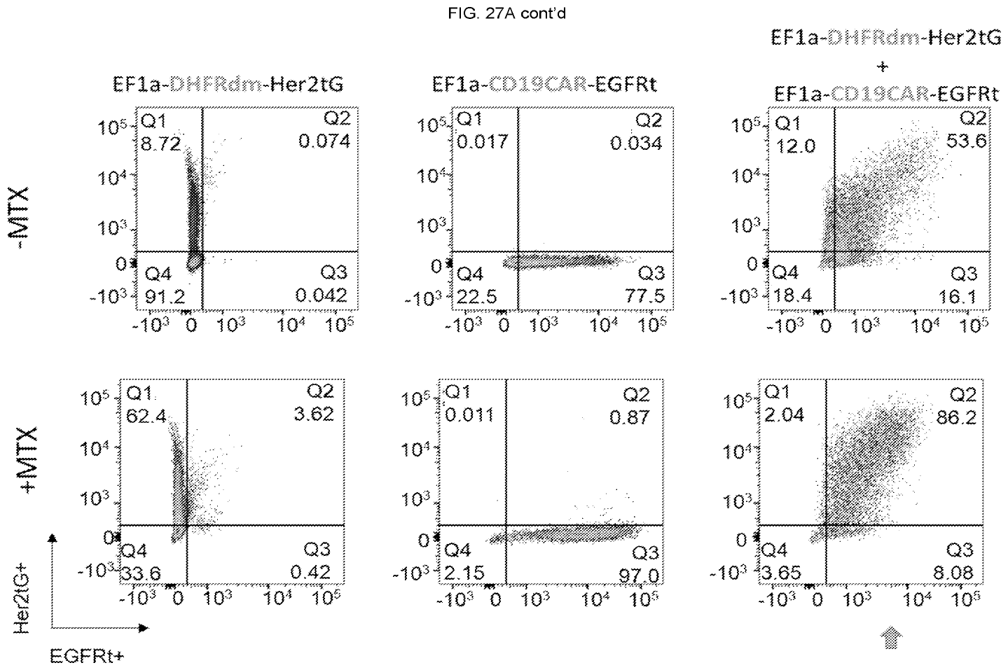

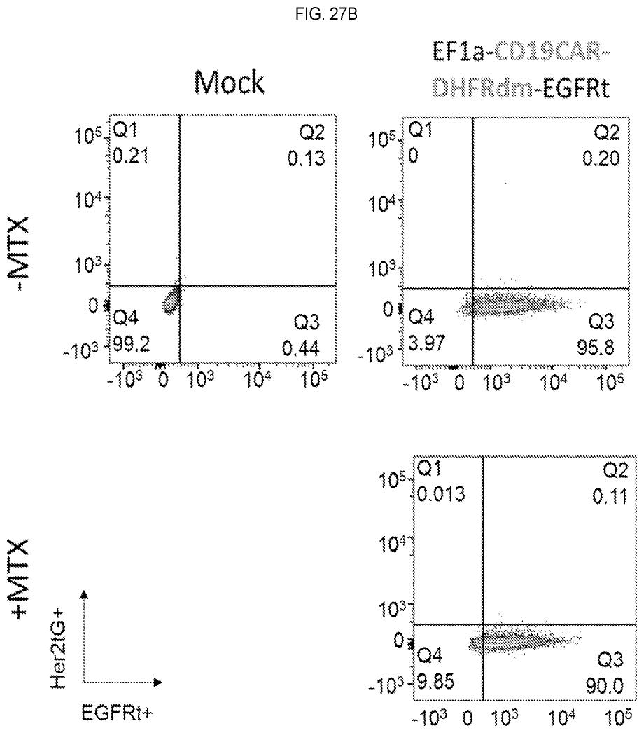

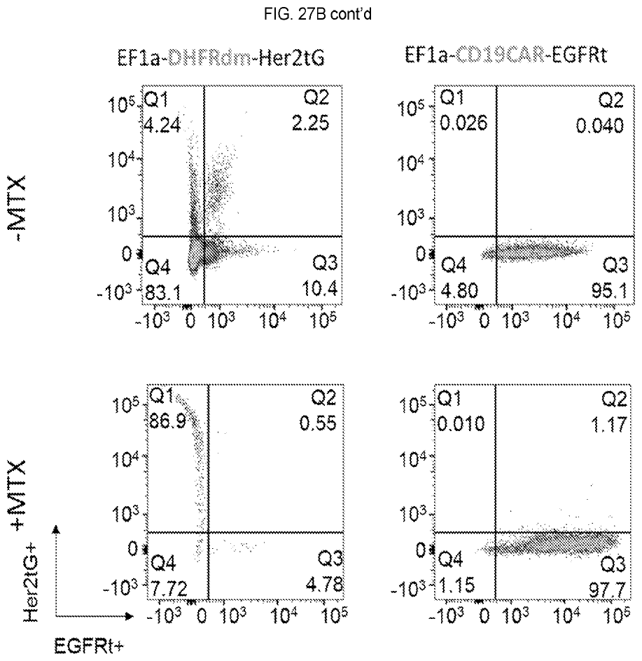

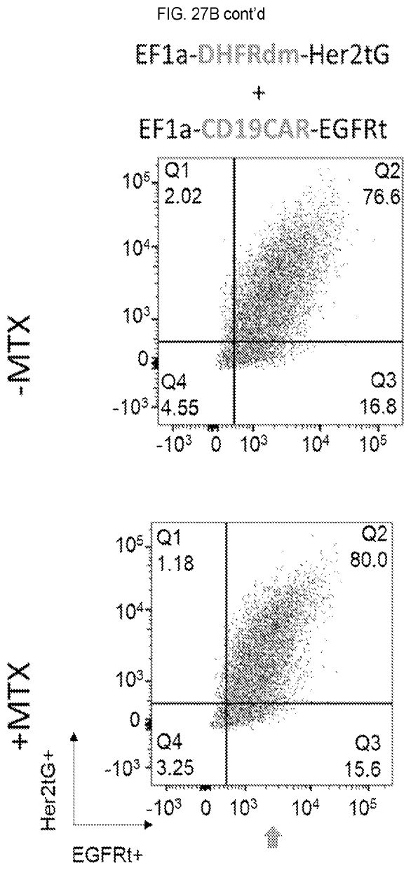

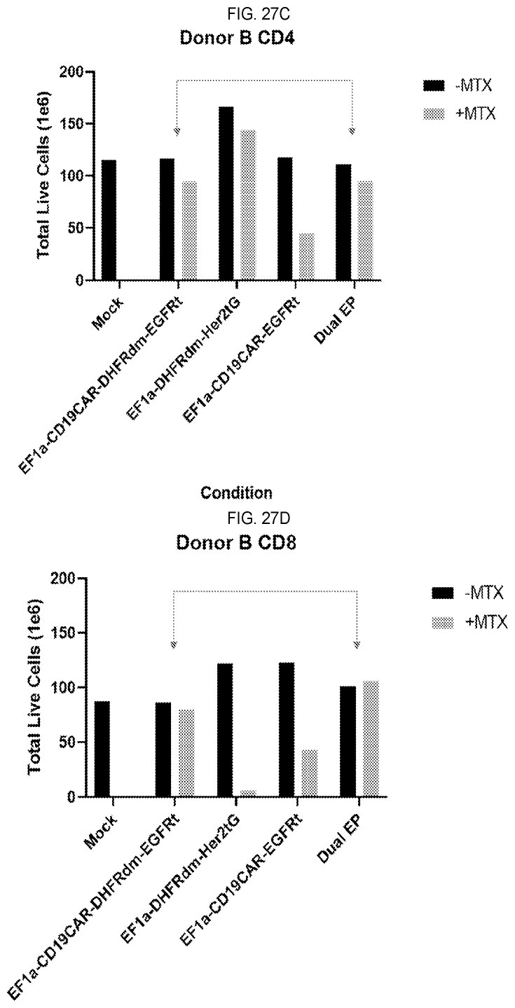

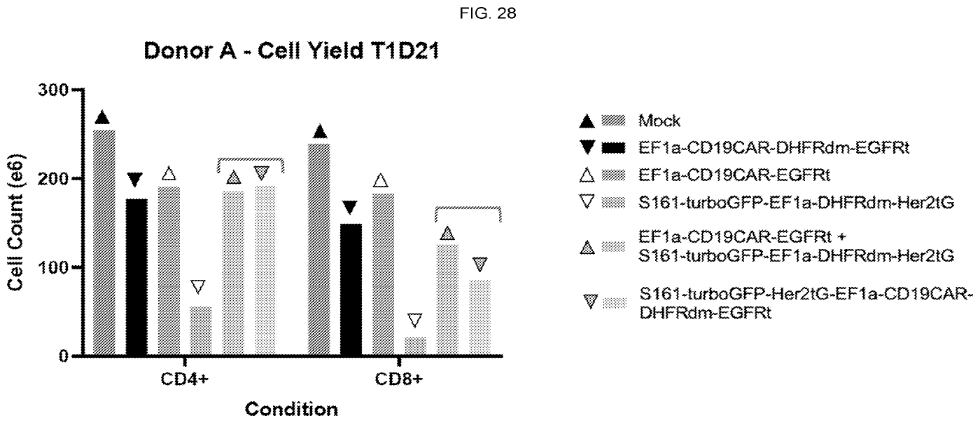

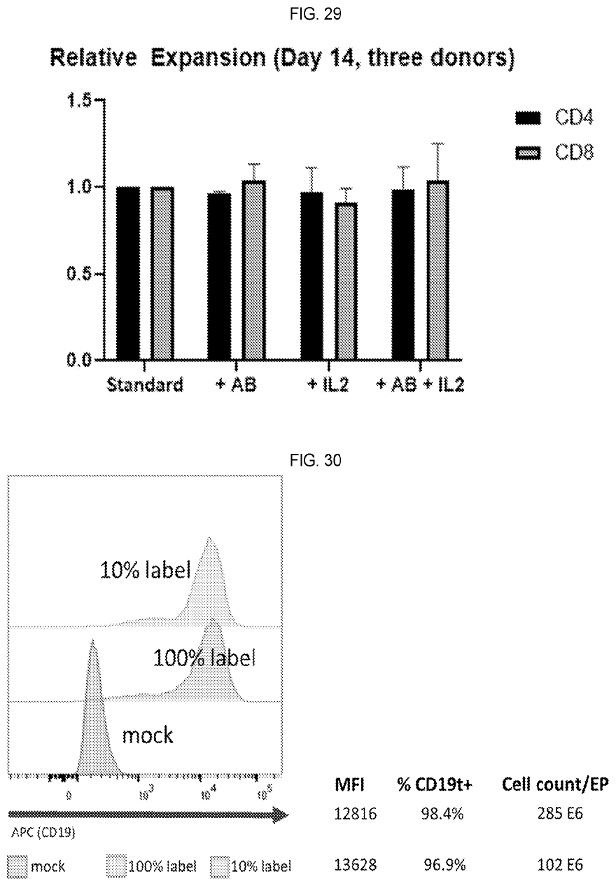

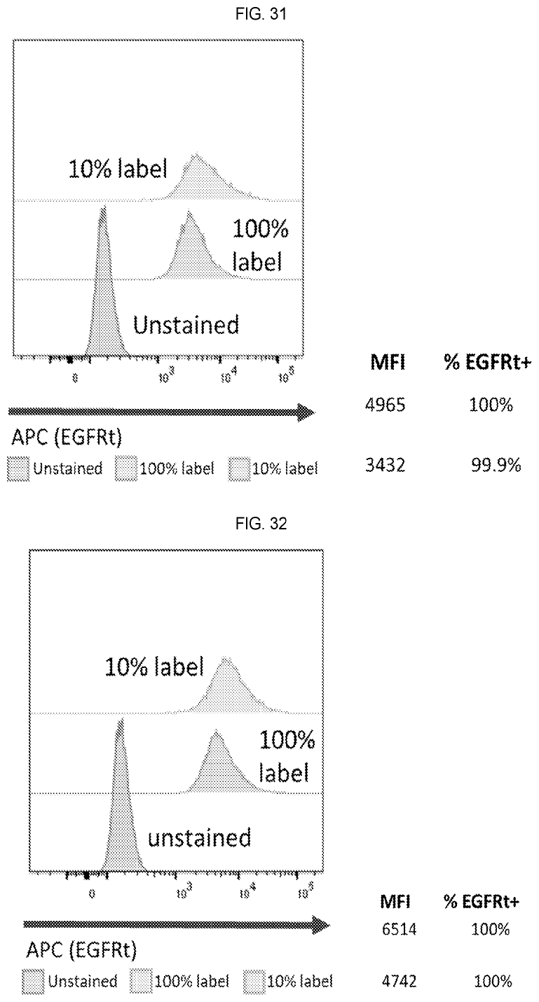

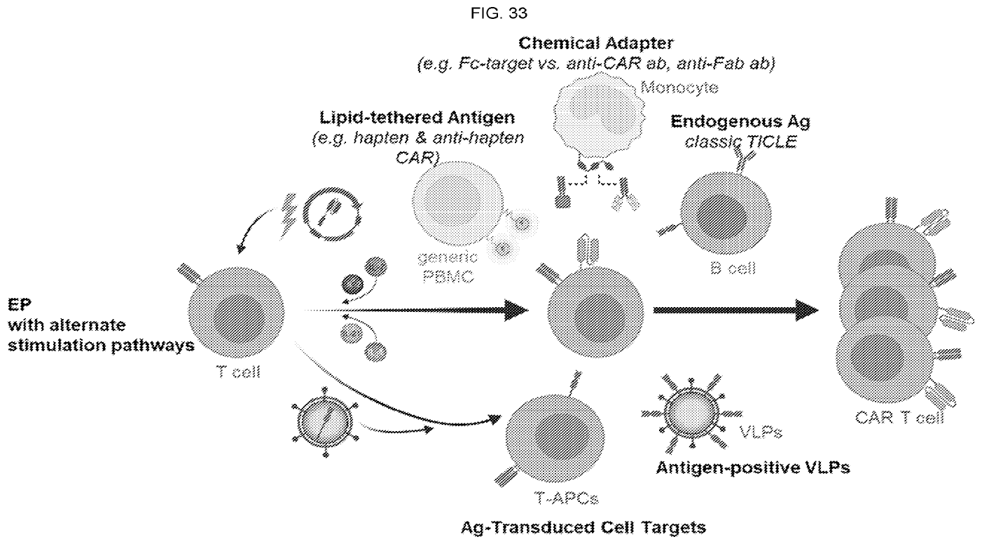

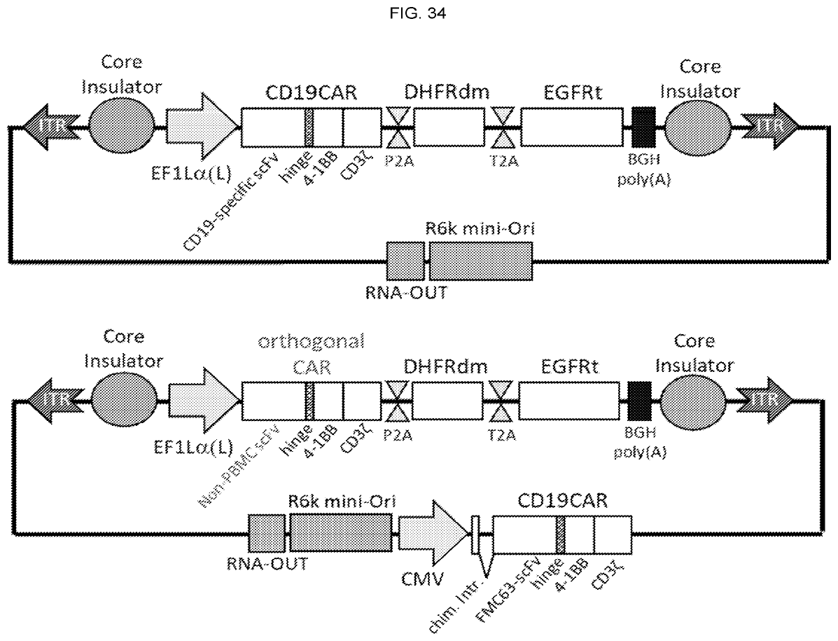

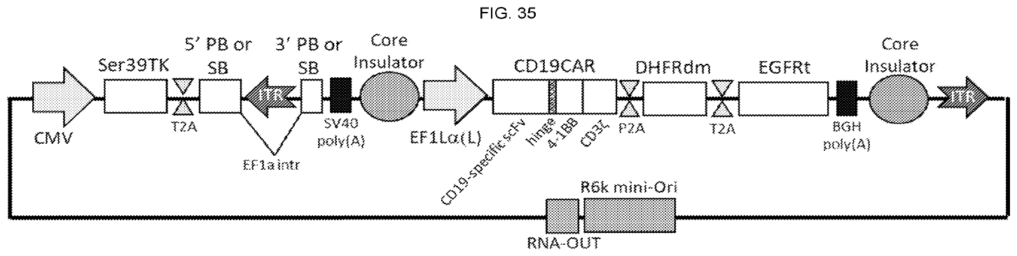

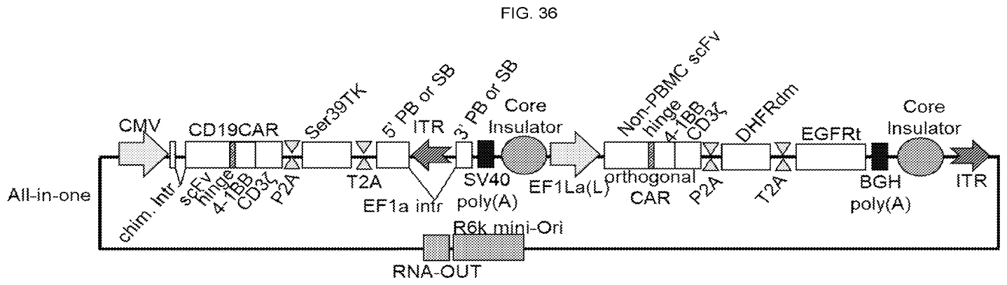

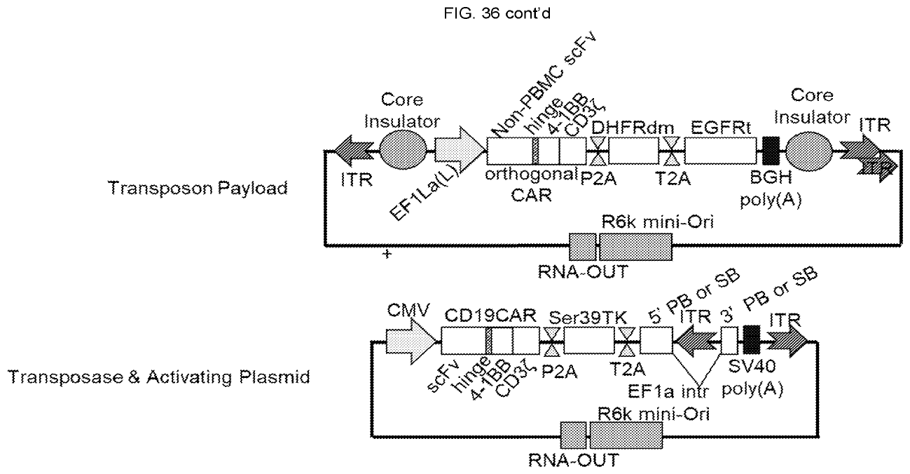

In certain examples, the introducing of the recombinant receptor-encoding construct into a T cell is through electroporation. Introduction of recombinant receptor-encoding constructs can include introduction of plasmid DNA and a transposase. In certain examples, the recombinant receptor is a CAR, and the CAR-encoding construct can be 15 kb in size. In other examples, the CAR-encoding construct can be 3.4-20 kb in size. As shown in FIG. 4, a CD19 CAR-encoding construct is 5.0 kb and includes or encodes an insulator, promoter, CAR-encoding sequence, transduction marker, and selection marker. Delivery and expression of larger CAR-encoding constructs, such as those shown in FIG. 5, were also achieved with the currently disclosed methods. These recombinant receptor-encoding constructs include at least two promoters, each promoter linked to expression of a different transgene, wherein at least one transgene encodes a recombinant receptor. Each of the promoters can be constitutive and/or inducible. Particularly useful promoters include iSynPro (e.g., SEQ ID NO: 25 of the current disclosure or SEQ ID NOs: 1-33 of WO2018/213332), MND, and EF1a (long or short).

Cell culture can be conducted in a gas-permeable vessel that supports cell expansion of concentrations of 10×10⁶ cells/mL or higher.

The methods can further include a recombinant receptor-expressing T cell enrichment step to purify the cell population. For example, certain recombinant receptor-encoding constructs include a drug-selection marker (also referred to herein as a selection cassette), allowing further cell purification within the population of cells. Exemplary drug selection markers include the DHFR (dihydrofolate reductase) gene or DHFR double mutant (DHFRdm) gene providing resistance to methotrexate (MTX).

Practicing the presently-disclosed methods, recombinant receptor-expressing T cell populations with greater than 80% and greater than 90% of T cells expressing the recombinant receptor were obtained. Further, over 50 million recombinant receptor-expressing T cells were obtained following introduction of the genetic constructs into only 2 million cells. In particular examples, over 75 million recombinant receptor-expressing T cells were obtained following introduction of the genetic constructs into only 2 million cells. In certain examples, over 100 million recombinant receptor-expressing T cells were obtained following introduction of the genetic constructs into only 2 million cells. More particularly, electroporation of 10×10⁶ CD8+ or CD4+ cells yielded 0.5×10⁹ or 10×10⁶ cells, respectively. Further, the expanded recombinant receptor-expressing T cell populations retained favorable, more naïve cell marker profiles, such as CD62L+/CD45RA+and CD27+/CD28+.

The methods disclosed herein allow selective manipulation of CD8+ or CD4+ cells with large, complex vectors, such as those depicted in FIG. 5. Such vectors can encode recombinant receptor-enhancing transgenes specific for the particular T cell lineage. Together the methods create beneficial recombinant receptor T cell products.

Aspects of the current disclosure are now described with additional details and options as follows: (i) Cell Types, Sources, and Isolation Techniques; (ii) Recombinant Receptors; (iii) Recombinant Receptor Encoding Genetic Constructs and Methods For Genetically Modifying Cells; (iv) Culture Conditions; (v) Ex Vivo Manufactured Cell Formulations; (vi) Methods of Use; (vii) Exemplary Embodiments; and (viii) Closing Paragraphs. These headings are provided for organizational purposes only do not limit the scope or interpretation of the disclosure.

### (i) Cell Types, Sources, and Isolation Techniques

The present disclosure provides methods for collecting, enriching for, culturing, and modifying cells to express a recombinant receptor.

Any source of T cells can be used within the methods of the disclosure. In some embodiments, T cells are derived or isolated from samples such as whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. In some aspects, the T cells are derived or isolated from blood or a blood-derived sample, or is derived from an apheresis product, a leukapheresis product, or a blood cone. The blood cone typically contains white blood cell components collected after plateletpheresis. It is similar to a leukapheresis product but collected and processed on a smaller scale. In relation to a particular subject, T cells can be autologous or allogeneic.

Autologous refers to any material (e.g., cells) derived from a subject wherein the material or a modified form thereof is later re-introduced to the same subject (i.e., the material is self-derived). Allogeneic refers to a material (e.g., cells) derived from one subject that is later administered (in original or modified form) to a different subject of the same species (i.e., the material is not self-derived).

In particular embodiments, lymphocytes are isolated from a sample such as blood or a blood-derived sample, an apheresis or a leukapheresis product. Exemplary samples include whole blood, peripheral blood mononuclear cells (PBMCs), bone marrow, thymus, cancer tissue, lymphoid tissue, spleen, or other appropriate sources. PBMCs refer to blood cells with round nuclei, such as monocytes, lymphocytes and macrophages. In humans, the majority of the PBMC population includes lymphocytes such as T cells, B cells, and NK cells.

In particular embodiments, cells from the circulating blood of an individual are obtained by apheresis. The apheresis product typically contains lymphocytes, including T cells, monocytes, granulocyte, B cells, other nucleated white blood cells, red blood cells, and platelets.

In particular embodiments, collected cells are washed, separated, enriched and/or cultured, for example, to remove unwanted components, enrich for desired components, or lyse or remove cells sensitive to particular reagents. In one embodiment, the cells collected by apheresis may be washed to remove the plasma fraction and to place the cells in an appropriate buffer or media for subsequent processing. The cells can be washed with phosphate buffered saline (PBS) or with another suitable solution that lacks calcium, magnesium, and most, if not all other, divalent cations. As would be appreciated by those of ordinary skill in the art, a washing step may be accomplished by methods known to those in the art, such as by using a semiautomated flow through centrifuge. For example, the Cobe 2991 cell processor, the Baxter Cyto Mate, or the like. After washing, the cells may be resuspended in a variety of biocompatible buffers or other saline solution with or without buffer.

The isolation can include one or more of various cell preparation and separation steps, including separation based on one or more properties, such as size, density, sensitivity or resistance to particular reagents, and/or affinity, e.g., immunoaffinity, to antibodies or other binding partners.

In particular embodiments, a sample can be enriched for T cells by using density-based cell separation methods and related methods. For example, white blood cells can be separated from other cell types in the peripheral blood by lysing red blood cells and centrifuging the sample through a Percoll or Ficoll gradient.

In particular embodiments, a bulk T cell population can be used that has not been enriched for a particular T cell type. In particular embodiments, one or more of the cell populations enriched, isolated and/or selected from a sample by the provided methods are cells that are positive for (marker+) or express high levels (marker^{hi}) of one or more particular markers, such as surface markers, or that are negative for (marker−) or express relatively low levels (marker^{lo}) of one or more markers. In positive selection, cells having bound cellular markers are retained for further use. In negative selection, cells not bound by a capture agent, such as an antibody to a cellular marker are retained for further use. In some examples, both fractions can be retained for a further use.

Several different subsets of T-cells have been discovered, each with a distinct function. For example, a majority of T-cells have a T-cell receptor (TCR) existing as a complex of several proteins. The actual T-cell receptor is composed of two separate peptide chains, which are produced from the independent T-cell receptor alpha and beta (TCRα and TCRβ) genes and are called α- and β-TCR chains.

γδT-cells represent a small subset of T-cells that possess a distinct T-cell receptor (TCR) on their surface. In γδT-cells, the TCR is made up of one γ-chain and one δ-chain. This group of T-cells is much less common (2% of total T-cells) than the αβ T-cells.

CD3 is expressed on all mature T cells. Activated T-cells express 4-1BB (CD137), CD69, and CD25.

T-cells can further be classified into helper cells (CD4⁺ T-cells) and cytotoxic T-cells (CTLs, CD8⁺ T-cells), which include cytolytic T-cells. T helper cells assist other white blood cells in immunologic processes, including maturation of B cells into plasma cells and activation of cytotoxic T-cells and macrophages, among other functions. These cells are also known as CD4⁺ T-cells because they express the CD4 protein on their surface. Helper T-cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T-cells destroy virally infected cells and tumor cells, and are also implicated in transplant rejection. These cells are also known as CD8+ T-cells because they express the CD8 glycoprotein on their surface. These cells recognize their targets by binding to antigen associated with MHC class I, which is present on the surface of nearly every cell of the body.

“Central memory” T-cells (or “TCM”) refers to an antigen experienced CTL that expresses CD62L or CCR7 and CD45RO on the surface thereof, and does not express or has decreased expression of CD45RA as compared to naive cells. In particular embodiments, central memory cells are positive for expression of CD62L, CCR7, CD25, CD127, CD45RO, and CD95, and have decreased expression of CD45RA as compared to naive cells.

“Effector memory” T-cell (or “TEM”) refers to an antigen experienced T-cell that does not express or has decreased expression of CD62L on the surface thereof as compared to central memory cells and does not express or has decreased expression of CD45RA as compared to a naive cell. In particular embodiments, effector memory cells are negative for expression of CD62L and CCR7, compared to naive cells or central memory cells, and have variable expression of CD28 and CD45RA. Effector T-cells are positive for granzyme B and perforin as compared to memory or naive T-cells.

“Naive” T-cells as refer to a non-antigen experienced T cell that expresses CD62L and CD45RA and does not express CD45RO as compared to central or effector memory cells. In particular embodiments, naive CD8+ T lymphocytes are characterized by the expression of phenotypic markers of naive T-cells including CD62L, CCR7, CD28, CD27, CD127, and CD45RA.

In particular embodiments, memory T cells show up-regulated gene expression of TCF7, LEF1, and CD27. In particular embodiments, memory T cells show down-regulated gene expression of NOTCH1, PRDM1, GZMB, PRF1, and EOMES.

B cells are mediators of the humoral response and are responsible for production and release of antibodies specific to an antigen. Several types of B cells exist which can be characterized by key markers. In general, immature B cells express CD19, CD20, CD34, CD38, and CD45R, and as they mature the key expressed markers are CD19 and IgM.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Herein, the “negative fraction” refers to the population of cells within a sample that remain after at least one positive selection has been performed. In particular embodiments, if a positive selection of CD8+ T cells was performed and a positive selection of CD4+ T cells was performed on a sample of PBMCs, the negative fraction includes the remaining PBMCs. In particular embodiments, if the positive selection of CD8+ T cells was performed on a sample of PBMCs, the negative fraction includes the remaining PBMCs (including CD4+ T cells). In particular embodiments, if the positive selection of CD4+ T cells was performed on a sample of PBMCs, the negative fraction includes the remaining PBMCs (including CD8+ T cells).

In particular embodiments, a CD8⁺ or CD4⁺ selection step is used to separate CD8⁺ cytotoxic T cells and CD4⁺ helper cells. Such CD8⁺ and CD4⁺ populations can be further sorted into sub-populations by positive or negative selection for markers expressed or expressed to a relatively higher degree on one or more naive, memory, and/or effector T cell subpopulations.

In some embodiments, enrichment for central memory T (T_{CM}) cells is carried out. In particular embodiments, memory T cells are present in both CD62L subsets of CD8⁺ peripheral blood lymphocytes. PBMC can be enriched for or depleted of CD62L, CD8 and/or CD62L⁺CD8⁺ fractions, such as by using anti-CD8 and anti-CD62L antibodies.

In some embodiments, the enrichment for central memory T (T_{CM}) cells is based on positive or high surface expression of CCR7, CD45RO, CD27, CD62L, CD28, CD3, and/or CD127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CCR7, CD45RO, and/or CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained, optionally following one or more further positive or negative selection steps.

In a particular example, a sample of PBMCs or other white blood cell sample is subjected to selection of CD8+ and/or CD4+ cells, where both the negative and positive fractions are retained.

Cell-markers for different T cell subpopulations are described above. In particular embodiments, specific subpopulations of T cells, such as cells positive or expressing high levels of one or more surface markers, e.g., CCR7, CD45RO, CD8, CD27, CD28, CD62L, CD127, CD4, and/or CD45RA T cells, are isolated by positive or negative selection techniques.

The separation need not result in 100% enrichment or removal of a particular cell population or cells expressing a particular marker. For example, positive selection of or enrichment for cells of a particular type refers to increasing the number or percentage of such cells but need not result in a complete absence of cells not expressing the marker. Likewise, negative selection, removal, or depletion of cells of a particular type refers to decreasing the number or percentage of such cells but need not result in a complete removal of all such cells.

In some examples, multiple rounds of separation steps are carried out, where the positively or negatively selected fraction from one step is subjected to another separation step, such as a subsequent positive or negative selection.

Any appropriate cell separation/isolation/purification method can be used. Exemplary techniques include using antibodies, flow cytometry, fluorescence activated cell sorting, filtration, gradient-based centrifugation, elution, microfluidics, immunomagnetic separation, multiple size immuno-beads filtration, fluorescent-magnetic separation, magnetic beads, nanostructure, quantum dots, high throughput microscope-based platform, or a combination thereof.

In some embodiments, an antibody or binding domain for a cellular marker is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher@ Humana Press Inc., Totowa, NJ); see also U.S. Pat. Nos. 4,452,773; 4,795,698; 5,200,084; and EP 452342.

In some embodiments, affinity-based selection is via magnetic-activated cell sorting (MACS) (Miltenyi Biotec, Auburn, CA). MACS systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1 (5): 355-376). In both cases, cells can be labeled with multiple markers, allowing for the isolation of well-defined T cell subsets at high purity.

### (ii) Recombinant Receptors

In particular embodiments, a recombinant receptor is or includes a binding domain that binds a target antigen, wherein the recombinant receptor is expressed by a cell following the artificial introduction of nucleic acid encoding the recombinant receptor into the cell. The recombinant receptor can be, e.g., a CAR, a T-cell receptor (TCR), or a CAR/TCR hybrid.

CAR, for example, include several distinct subcomponents that allow genetically modified cells (e.g., T cells) to recognize and kill target cells. The subcomponents include at least an extracellular component and an intracellular component. The extracellular component includes a binding domain that binds a target antigen. In particular embodiments, the target antigen can be present on the surface of target cells. When the binding domain binds such antigens, the intracellular component activates the cell to destroy the bound cell. CAR additionally include a transmembrane domain that directly or indirectly links the extracellular component to the intracellular component, and other subcomponents that can increase the CAR's function. For example, the inclusion of a spacer region and/or one or more linker sequences can allow the CAR to have additional conformational flexibility, often increasing the binding domain's ability to bind the targeted antigen. In particular embodiments, an extracellular binding domain is any molecule capable of specifically binding a target antigen. Exemplary binding domains include antibodies or binding fragments thereof, receptors (e.g., T cell receptors), and receptor ligands (e.g., a cytokine or chemokine).

As is understood by those of ordinary skill in the art, a complete antibody includes two heavy chains and two light chains. Each heavy chain consists of a variable region and a first, second, and third constant region, while each light chain consists of a variable region and a constant region. Mammalian heavy chains are classified as α, δ, ε, γ, and μ, and mammalian light chains are classified as λ or κ. Immunoglobulins including the α, δ, ε, γ, and μ heavy chains are classified as immunoglobulin (Ig) A, IgD, IgE, IgG, and IgM. The complete antibody forms a “Y” shape. The stem of the Y consists of the second and third constant regions (and for IgE and IgM, the fourth constant region) of two heavy chains bound together and disulfide bonds (inter-chain) are formed in the hinge. Heavy chains γ, α and δ have a constant region composed of three tandem (in a line) Ig domains, and a hinge region for added flexibility; heavy chains μ and ε have a constant region composed of four immunoglobulin domains. The second and third constant regions are referred to as “CH2 domain” and “CH3 domain”, respectively. Each arm of the Y includes the variable region and first constant region of a single heavy chain bound to the variable and constant regions of a single light chain. The variable regions of the light and heavy chains are responsible for antigen binding.

Light and heavy chain variable regions contain a “framework” region interrupted by three hypervariable regions, also called “complementarity determining regions” or “CDRs”.

CDR sets can be based on, for example, Kabat numbering (Kabat et al. (1991) “Sequences of Proteins of Immunological Interest,” 5th Ed. Public Health Service, National Institutes of Health, Bethesda, Md. (“Kabat” numbering scheme)); Chothia (Al-Lazikani et al. (1997) JMB 273:927-948 (“Chothia” numbering scheme)); Martin (Abinandan et al. (2008) Mol Immunol. 45:3832-3839 (“Martin” numbering scheme)); Gelfand (Gelfand and Kister (1995) Proc Natl Acad Sci USA. 92:10884-10888; Gelfand et al. (1998) Protein Eng. 11:1015-1025; Gelfand et al. (1996) Proc Natl Acad Sci USA. 93:3675-3678; Gelfand et al. (1998) J Comput Biol. 5:467-477 (“Gelfand” numbering scheme)); Contact (MacCallum et al. (1996) J. Mol. Biol. 262:732-745 (Contact numbering scheme)); IMGT (Lefranc et al. (2003) Dev Comp Immunol 27 (1): 55-77 (“IMGT” numbering scheme)); AHo (Honegger and Plückthun (2001) J Mol Biol 309 (3): 657-670 (“AHo” numbering scheme)); North (North et al. (2011) J Mol Biol. 406 (2): 228-256 (“North” numbering scheme)); or other numbering schemes.

Software programs and bioinformatical tools, such as ABodyBuilder (Leem et al. (2016) MAbs 8 (7): 1259-1268), PIGSPro (Lepore et al. (2017) Nucleic Acids Res 45 (W1): W17-W23), Kotai Antibody Builder (Yamashita et al. (2014) Bioinformatics 30 (22): 3279-3280), Rosetta Antibody (Weitzner et al. (2017) Nature Protocols 12:401-416), Paratome (Kunik et al. (2012) Nucleic Acids Res 40: W521-W524), Antibody i-Patch (Krawczyk et al. (2013) Protein Eng Des Sel 26 (10): 621-629), and proABC-2 (Ambrosetti et al. (2020) Bioinformatics 36 (20): 5107-5108 can also be used to determine CDR sequences.

The sequences of the framework regions of different light or heavy chains are relatively conserved within a species, such as humans. The framework region of an antibody, that is the combined framework regions of the constituent light and heavy chains, serves to position and align the CDRs in three-dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, the CDRs located in the variable domain of the heavy chain of the antibody are referred to as CDRH1, CDRH2, and CDRH3, whereas the CDRs located in the variable domain of the light chain of the antibody are referred to as CDRL1, CDRL2, and CDRL3. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain. References to “V_{L}” or “VL” refer to the variable region of an immunoglobulin light chain.

Antibodies that specifically bind an antigen can be prepared using methods of obtaining monoclonal antibodies, methods of phage display, methods to generate human or humanized antibodies, or methods using a transgenic animal or plant engineered to produce human antibodies. Phage display libraries of partially or fully synthetic antibodies are available and can be screened for an antibody or fragment thereof that can bind to the target antigen. Phage display libraries of human antibodies are also available. Once identified, the amino acid sequence or polynucleotide sequence coding for the antibody can be isolated and/or determined. Many relevant antibodies are also publicly known and commercially available.

In some alternatives, antibodies specifically bind to a cancer cell or virally-infected cell surface molecule and do not cross react with nonspecific components such as bovine serum albumin or other unrelated antigens.

“Antibody fragment” refers to at least one portion of an antibody, that retains the ability to specifically binding an antigen. Examples of antibody fragments include Fab, Fab′, F(ab′)₂, Fv fragments, single chain variable (scFv) antibody fragments, disulfide-linked Fvs (sdFv), a Fd fragment including VH and constant CH1 domains, linear antibodies, single domain antibodies such as sdAb (either VL or VH), camelid variable heavy only (VHH) domains, multi-specific antibodies formed from antibody fragments such as a bivalent fragment including two Fab fragments linked by a disulfide bridge at the hinge region, and an isolated CDR or other epitope binding fragments of an antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson (2005) Nature Biotechnology 23:1126-1136).

In particular embodiments, a binding domain can include humanized forms of non-human (e.g., murine) antibodies or antigen binding fragments thereof. A humanized antibody includes an antibody in which the constant and variable framework region of one or more human immunoglobulins is fused with the binding region, e.g., the CDR, of an animal (non-human) immunoglobulin. Such humanized antibodies are designed to maintain the binding specificity of the non-human antibody from which the binding regions are derived but avoid an immune reaction against the non-human antibody. In particular embodiments, a binding domain can include a fully human antibody or antibody fragment thereof, where the whole molecule is of human origin or includes an amino acid sequence identical to a human form of the antibody or immunoglobulin.

“scFv” refers to an engineered fusion protein including the VH and VL of an antibody linked via a linker and capable of being expressed as a single chain polypeptide. The scFv retains the specificity of the intact antibody from which it is derived. In particular embodiments, a linker connecting the variable regions can include glycine-serine linkers, including, for example, those shown as SEQ ID NOs: 53-66 or described elsewhere herein. In particular embodiments, an scFv may have the VL and VH variable regions in either order, e.g., with respect to the N-terminal and C-terminal ends of the polypeptide, the scFv may include VL-linker-VH or may include VH-linker-VL.

Recombinant receptors also include TCR that can be used independently or as a binding domain within a CAR (e.g., CAR/TCR hybrid). For example, the sequences of numerous TCR that bind particular antigen fragments are known and publicly available.

TCR can also be identified for use with a particular antigen by, for example, isolating T cells that bind a particular antigen/MHC complex and sequencing the TCR chains binding the complex. TCR genes encoding TCR can be readily cloned by, for example, the 5′ RACE procedure using primers corresponding to the sequences specific to the TCR α-chain gene and the TCR β-chain gene.

In particular embodiments, it may be necessary to pair TCR chains following sequencing (i.e., to perform paired chain analysis). Various methods can be utilized to pair chains, when necessary. For example, chain pairing may be assisted in silico by computer methods, such as immunology gene alignment software available from IMGT, JOINSOLVER, VDJSolver, SoDA, iHMMune-align, or other similar tools for annotating VDJ gene segments. Assays such as PairSEQ® (Adaptive Biotechnologies Corp., Seattle, WA) have also been developed.

In particular embodiments, an engineered TCR includes a single chain T cell receptor (scTCR) including Va/β and Ca/β chains (e.g., Va-Cα, Vβ-Cβ, Vα-Vβ) or including Vα-Cα, Vβ-Cβ, Vα-Vβ pair specific for a target of interest (e.g., peptide-MHC complex).

In particular embodiments, a CAR/TCR hybrid includes components of a CAR and components of a TCR. For example, a CAR/TCR hybrid can include a TCR binding domain and intracellular signaling domains specific to CAR. This allows for antigen recognition similar to that of a TCR but use of intracellular signaling similar to a CAR.

Cancer antigens are proteins that are produced by cancer cells and viral antigens are proteins produced by virally-infected cells. Binding domains of recombinant receptors disclosed herein can be selected to bind cancer antigens or viral antigens. In some alternatives, cancer or viral antigens are selectively expressed or overexpressed on the cancerous or infected cells as compared to other cells of the same tissue type. In some alternatives, a cancer or viral antigen is a cell surface molecule that is found on cancer cells or virally-infected cells and is not substantially found on normal tissues, or restricted in its expression to non-vital normal tissues.

In particular embodiments, cancer antigens or viral antigens are preferentially expressed by cancer cells or virally-infected cells, respectively. “Preferentially expressed” means that the antigen is found on the targeted cell type at least 25%, 35%, 45%, 55%, 65%, 75%, 85%, 95%, 96%, 97%, 98%, 99%, or 100% more than on non-targeted cells.

Exemplary cancer antigens include carcinoembryonic antigen (CEA), prostate specific antigen, Prostate Stem Cell antigen (PSCA), PSMA, Her2/neu, estrogen receptor, progesterone receptor, ephrinB2, CD19, CD20, CD22, CD23, CD123, CS-1, CE7, ROR1, mesothelin, c-Met, GD-2, MAGE A3 TCR, EGFR, EGFRvIII, EphA2, IL13Ra2, L1CAM, oaGD2, GD2, B7H3, CD33, FITC, VAR2CSA, MUC16, PD-L1, ERBB2, folate receptor (FOLR), CD56; glypican-2, disialoganglioside, EpCam, L1-CAM, Lewis Y, WT-1, Tyrosinase related protein 1 (TYRP1/gp75); GD2, B-cell maturation antigen (BCMA), CD24, SV40 T, carboxy-anhydrase-IX (CAIX); and CD133. Other examples are known to those of ordinary skill in the art. Particular embodiments utilize binding domains that specifically bind CD19.

In particular embodiments, a binding domain that binds a cancer antigen includes an scFv. In particular embodiments, an scFv includes an huCD19 (G01S) scFv, a muCD19 (FMC63) scFv, a CD20 (Leu 16) scFv, a CD22 (m971) scFv, a B7H3 (hBRCA84D) scFv, an L1CAM (CE7) scFv, an EGFR scFv, an EGFRVIII (806) scFv, an EphA2 (2A4) scFv, an EpHA2 (4H5) scFv, an FITC (E2) scFv, a GD2 (hu3F8) scFv, a Her2 (Herceptin) scFv, an IL13Ra2 (hu08) VIVh scFv, an IL13Ra2 hu08 VhV1 scFv, an IL13Ra2 (hu07) VhV1 scFv, an IL13Ra2 (hu07) VhVI scFv, an oaGD2 (8B6) VIVh, a ROR1 (R12) scFv, a CD33 (h2H12) VhVI scFv, a CD33 (h2H12) VIVh scFv, a mesothelin (P4) scFv, a VAR2CSA (ID1-DBL2Xb) scFv, or an IL13Ra2 (IL13 zetakine) amino acid sequence.

In particular embodiments, binding domains that bind CD20 can be based on the binding domains of SP32 (ab64088), EP459Y (ab78237), rIGEL/773 (ab219329), ocrelizumab, rituximab, ofatumumab, obinutuzumab), ibritumomab, or tositumomab.

Exemplary viral antigens include coronaviral antigens: the spike(S) protein; cytomegaloviral antigens: envelope glycoprotein B and CMV pp65; Epstein-Barr antigens: EBV EBNAI, EBV P18, and EBV P23; hepatitis antigens: the S, M, and L proteins of hepatitis B virus, the pre-S antigen of hepatitis B virus, HBCAG DELTA, HBV HBE, hepatitis C viral RNA, HCV NS3 and HCV NS4; herpes simplex viral antigens: immediate early proteins and glycoprotein D; HIV antigens: gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; influenza antigens: hemagglutinin and neuraminidase; Japanese encephalitis viral antigens: proteins E, M-E, M-E-NS1, NS1, NS1-NS2A and 80% E; measles antigens: the measles virus fusion protein; rabies antigens: rabies glycoprotein and rabies nucleoprotein; respiratory syncytial viral antigens: the RSV fusion protein and the M2 protein; rotaviral antigens: VP7sc; rubella antigens: proteins E1 and E2; and varicella zoster viral antigens: gpI and gpII. See Fundamental Virology, Second Edition, eds. Fields, B. N. and Knipe, D. M. (Raven Press, New York, 1991) for additional examples of viral antigens. In particular embodiments, binding domains that bind viral antigens can be used.

In particular embodiments, a bacterial antigen includes antigens expressed by bacteria. In particular embodiments, a fungal antigen includes antigens expressed by fungi. In particular embodiments, an arthropod antigen includes antigens expressed by an arthropod.

In particular embodiments, a binding domain binds an immune cell activating epitope. In particular embodiments, an immune cell activating epitope is part of an antigen. In particular embodiments, the immune cell activating epitope can be expressed by an immune cell within the negative fraction of a sample and/or can be a binding domain on a multi-specific binding molecule (also referred to as a chemical adapter). An immune cell activating epitope is a portion of a molecule (e.g., portion of a protein) that activates an immune cell when bound by a binding domain expressed by the immune cell. An immune cell activating epitope can be on anything that binds the recombinant receptor and links the bound T cell to an activating cell (e.g., PBMCs). In certain examples, a chemical adapter is an antibody.

In particular embodiments, the immune cell activating epitope can be a B-cell ligand, wherein the B-cell ligand is CD1d, CD5, CD19, CD20, CD21, CD22, CD23/Fc epsilon RII, CD24, CD25/IL-2 R alphaCD27/TNFRSF7, CD32, CD34, CD35, CD38, CD40 (TNFRSF5), CD44, CD45, CD45.1, CD45.2, CD54 (ICAM-1), CD69, CD72, CD79, CD80, CD84/SLAMF5, LFA-1, CALLA, BCMA, B-cell receptor (BCR), IgMs, IgD, B220/CD45R, C1q R1/CD93, CD84/SLAMF5, BAFF R/TNFRSF13C, B220/CD45R, B7-1/CD80, B7-2/CD86, TNFSF7, TNFRSF5, ENPP-1, HVEM/TNFRSF14, BLIMP 1/PRDM1, CXCR4, DEP-1/CD148 or EMMPRIN/CD 147.

Other immune cell activating epitopes can be found on, e.g., natural killer T (NKT) cells, natural killer cells (also known as K cells and killer cells), tumor-infiltrating lymphocytes (TILs), marrow-infiltrating lymphocytes (MILs), MAIT cells, macrophages, monocytes, and/or dendritic cells. These cells and exemplary cell surface antigens are described elsewhere herein.

In particular embodiments, the immune cell activating epitope is a hapten. Haptens include any small molecule which, when combined with a larger carrier such as a protein, elicits the production of antibodies which bind specifically to it (in the free or combined state). Haptens can include peptides, other larger chemicals, and aptamers. In some embodiments, a hapten can be any hapten provided in the hapten database accessible on the World Wide Web under the URL crdd.osdd.net/raghava/haptendb/. In particular embodiments, the hapten is tethered to an activating cell. In particular embodiments, if the immune cell activating epitope is a hapten, the cell expressing the recombinant receptor includes a binding domain that binds the hapten. In particular embodiments, the binding domain that binds the hapten can be the binding domain of the recombinant receptor. In particular embodiments, the binding domain that binds the hapten can be a binding domain not on the recombinant receptor.

In particular embodiments, the immune cell activating epitope can be a binding domain of a multi-specific binding molecule (also referred to as a chemical adapter). In particular embodiments, a multi-specific binding molecule includes at least two binding domains wherein at least one binding domain is the immune cell activating epitope that binds the T cell expressing a recombinant receptor and at least one binding domain binds an immune cell within the negative fraction. Multi-specific binding molecules that are useful for T cell activation are described elsewhere herein. In particular embodiments, multi-specific binding molecules include bispecific antibodies. In particular embodiments, multi-specific binding molecules include antibodies. An antibody can be considered a multi-specific binding molecule because the antigen binding domain binds an antigen while the Fc interacts with immune cells within the negative fraction.

An intracellular component of a recombinant receptor can include one or more intracellular signaling domains. In particular embodiments, the intracellular signaling domain generates a signal that promotes an immune effector function of a recombinant receptor-modified cell. In particular embodiments, the intracellular signaling domain generates a stimulatory and/or co-stimulatory signal based on binding to a target antigen. Examples of immune effector function include cytolytic activity and helper activity, including the secretion of cytokines. Intracellular signaling domain signals can also lead to immune cell proliferation, activation, differentiation, and the like.

A signaling domain refers to the functional portion of a protein which acts by transmitting information within the cell to regulate cellular activity via defined signaling pathways by generating second messengers or functioning as effectors by responding to such messengers. Stimulation refers to a primary response induced by binding of a stimulatory molecule (e.g., a CAR) or co-stimulatory molecule with its cognate ligand, thereby mediating a signal transduction event, such as signal transduction via appropriate signaling domains of the recombinant receptor. Stimulation can mediate altered expression of certain molecules.

An intracellular signaling domain can include the entire intracellular portion of the signaling domain or a functional fragment thereof. In particular embodiments, an intracellular signaling domain can include a primary intracellular signaling domain. In particular embodiments, primary intracellular signaling domains include those derived from the molecules responsible for primary stimulation, or antigen dependent stimulation. In particular embodiments, the intracellular signaling domain can include a costimulatory intracellular domain.

A primary intracellular signaling domain can include a signaling motif which is known as an immunoreceptor tyrosine-based activation motif or ITAM. Examples of ITAM containing primary cytoplasmic signaling sequences include those derived from CD3ζ, common FcR gamma (FCER1G), Fc gamma RIIa, FcR beta (Fc Epsilon R1b), CD3 gamma, CD3 delta, CD3 epsilon, CD79a, CD79b, DAP10, and DAP12. In particular embodiments, variants of CD3ζ retain at least one, two, three, or all ITAM regions.

In particular embodiments, a CD3ζ (CD247) stimulatory domain can include amino acid residues from the cytoplasmic domain of the T cell receptor zeta chain, or functional fragments thereof, that are sufficient to functionally transmit an initial signal necessary for cell activation. In particular embodiments, a CD3ζ stimulatory domain can include a human CD3ζ stimulatory domain or functional fragments thereof. In particular embodiments, a CD3ζ stimulatory domain includes SEQ ID NO: 10. In particular embodiments, a CD3ζ stimulatory domain includes SEQ ID NO: 17. In particular embodiments, in the case of an intracellular signaling domain that is derived from a CD3ζ molecule, the intracellular signaling domain retains sufficient CD3ζ structure such that it can generate a signal under appropriate conditions.

In particular embodiments, the intracellular signaling domain can include a costimulatory intracellular domain. In particular embodiments, costimulatory intracellular signaling domains include those derived from molecules responsible for costimulatory signals, or antigen independent stimulation. In particular embodiments, a costimulatory intracellular signaling domain can be the intracellular portion of a costimulatory molecule. A costimulatory molecule refers to a cognate binding partner on an immune cell that binds with a costimulatory ligand, thereby mediating a costimulatory response by the immune cell, such as proliferation. Costimulatory molecules include cell surface molecules other than antigen receptors or their ligands that contribute to an efficient immune response. A costimulatory molecule can be represented in the following protein families: TNF receptor proteins, immunoglobulin-like proteins, cytokine receptors, integrins, signaling lymphocytic activation molecules (SLAM proteins), and activating NK cell receptors. Examples of such molecules include: an MHC class I molecule, B and T cell lymphocyte attenuator (BTLA, CD272), a Toll ligand receptor, CD27, CD28, 4-1BB (CD137), OX40, GITR, CD30, CD40, ICOS (CD278), BAFFR, HVEM (LIGHTR), ICAM-1, lymphocyte function-associated antigen-1 (LFA-1; CD11a/CD18), CD2, CDS, CD7, CD287, LIGHT, NKG2C, NKG2D, SLAMF7, NKp80 (KLRF1), NKp30, NKp44, NKp46, CD160 (BY55), B7-H3 (CD276), CD19, CD4, CD8α, CD8β, IL2Rβ, IL2Rγ, IL7Rα, ITGA4, VLA1, CD49a, IA4, CD49d, ITGA6, VLA-6, CD49f, ITGAD, CD11d, ITGAE, CD103, ITGAL, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that binds with CD83, and the like.

In particular embodiments, a costimulatory intracellular signaling domain includes CD27, CD28, 4-1BB (CD 137), OX40, PD-1, ICOS, lymphocyte function-associated antigen-1 (LFA-1), NKG2C, and a ligand that binds with CD83.

In particular embodiments, the amino acid sequence of the intracellular signaling component includes a variant of CD3ζ (SEQ ID NO: 10) and a portion of the 4-1BB (SEQ ID NO: 9) intracellular signaling component.

Recombinant receptors can be designed to include a transmembrane domain that links an extracellular component of the recombinant receptor to an intracellular component of the recombinant receptor when expressed. A transmembrane domain can anchor a recombinant receptor to a cell membrane. A transmembrane domain can include one or more additional amino acids adjacent to the transmembrane region, e.g., one or more amino acids associated with the extracellular region of the protein from which the transmembrane was derived (e.g., 1, 2, 3, 4, 5, 6, 7, 8, 9, 10, 11, 12, 13, 14, 15 amino acids, or more of the extracellular region) and/or one or more additional amino acids associated with the intracellular region of the protein from which the transmembrane protein is derived (e.g., 1, 2, 3, 4, 5, 6, 7, 8, 9, 10, 11, 12, 13, 14, 15 amino acids, or more of the intracellular region). In particular embodiments, the transmembrane domain may be from the same protein that the signaling domain, costimulatory domain, or hinge domain is derived from. In particular embodiments, the transmembrane domain is not derived from the same protein that any other domain of a fusion protein is derived from. In particular embodiments, the transmembrane domain can be selected or modified by amino acid substitution to avoid binding of or to minimize interactions with other domains in the fusion protein.

Transmembrane domains typically have a three-dimensional structure that is thermodynamically stable in a cell membrane, and generally ranges in length from 15 to 30 amino acids. The structure of a transmembrane domain can include an a helix, a β barrel, a β sheet, a β helix, or any combination thereof.

A transmembrane domain can include one or more additional amino acids adjacent to the transmembrane region, e.g., one or more amino acid within the extracellular region of the recombinant receptor (e.g., up to 15 amino acids of the extracellular region) and/or one or more additional amino acids within the intracellular region of the recombinant receptor (e.g., up to 15 amino acids of the intracellular components).

The transmembrane domain can be derived either from a natural and/or a synthetic source. When the source is natural, the transmembrane domain can be derived from any membrane-bound or transmembrane protein. Transmembrane domains can include at least the transmembrane region(s) of the α, β or ζ chain of a T-cell receptor, CD28, CD27, CD3 epsilon, CD45, CD4, CD5, CD8, CD9, CD16, CD22; CD33, CD37, CD64, CD80, CD86, CD134, CD137 and CD154. In particular embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7R a, ITGA1, VLA1, CD49a, ITGA4, IA4, CD49D, ITGA6, VLA-6, CD49f, ITGAD, CDI Id, ITGAE, CD103, ITGAL, CDI Ia, ITGAM, CDI Ib, ITGAX, CDI Ic, ITGB1, CD29, ITGB2, CD18, ITGB7, TNFR2, DNAM1 (CD226), SLAMF4 (CD244, 2B4), CD84, CD96 (Tactile), CEACAM1, CRT AM, Ly9 (CD229), PSGL1, CD100 (SEMA4D), SLAMF6 (NTB-A, Lyl08), SLAM (SLAMF1, CD150, IPO-3), BLAME (SLAMF8), SELPLG (CD162), LTBR, PAG/Cbp, NKG2D, or NKG2C. In particular embodiments, the transmembrane domains can include at least the transmembrane region(s) of the α, β or ζ chain of a T-cell receptor, CD28, CD27, CD3, CD45, CD4, CD5, CD8, CD9, CD16, CD22; CD33, CD37, CD64, CD80, CD86, CD134, CD137 or CD154.

In particular embodiments, a CD28 transmembrane domain is set forth in SEQ ID NO: 11.

In particular embodiments, the transmembrane domain can include predominantly hydrophobic residues such as leucine and valine. In particular embodiments, the transmembrane domain can include a triplet of phenylalanine, tryptophan and valine found at each end of the transmembrane domain. In particular embodiments, a CD28 or CD8 hinge is juxtaposed on the extracellular side of the transmembrane domain.

A linker within a recombinant receptor can be any portion of a recombinant receptor that serves to connect two subcomponents or domains of the recombinant receptor. In particular embodiments, linkers can provide flexibility for different components of the recombinant receptor. Linkers can also include spacer regions and junction amino acids. In certain examples, when a more rigid linker is required, proline-rich linkers can be used.

Spacer regions are used to create appropriate distances and/or flexibility from other recombinant receptor sub-components. Spacer regions typically include 10 to 250 amino acids, 10 to 200 amino acids, 10 to 150 amino acids, 10 to 100 amino acids, 10 to 50 amino acids, or 10 to 25 amino acids. Exemplary spacer regions include all or a portion of an immunoglobulin hinge region.

Exemplary spacer regions include all or a portion of an immunoglobulin hinge region. An immunoglobulin hinge region may be a wild-type immunoglobulin hinge region or an altered wild-type immunoglobulin hinge region. In certain embodiments, an immunoglobulin hinge region is a human immunoglobulin hinge region. A “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

An immunoglobulin hinge region may be an IgG, IgA, IgD, IgE, or IgM hinge region. An IgG hinge region may be an IgG1, IgG2, IgG3, or IgG4 hinge region. Sequences from IgG1, IgG2, IgG3, IgG4 or IgD can be used alone or in combination with all or a portion of a CH2 region; all or a portion of a CH3 region; or all or a portion of a CH2 region and all or a portion of a CH3 region. In particular embodiments, an IgG4 hinge region is set forth in SEQ ID NO: 16.

Other examples of hinge regions used in fusion binding proteins described herein include the hinge region present in the extracellular regions of type 1 membrane proteins, such as CD8α, CD4, CD28 and CD7, which may be wild-type or variants thereof.

In particular embodiments, a spacer region includes a hinge region that includes a type II C-lectin interdomain (stalk) region or a cluster of differentiation (CD) molecule stalk region. A “stalk region” of a type II C-lectin or CD molecule refers to the portion of the extracellular domain of the type II C-lectin or CD molecule that is located between the C-type lectin-like domain (CTLD; e.g., similar to CTLD of natural killer cell receptors) and the hydrophobic portion (transmembrane domain). For example, the extracellular domain of human CD94 (GenBank Accession No. AAC50291.1) corresponds to amino acid residues 34-179, but the CTLD corresponds to amino acid residues 61-176, so the stalk region of the human CD94 molecule includes amino acid residues 34-60, which are located between the hydrophobic portion (transmembrane domain) and CTLD (see Boyington et al., Immunity 10:15, 1999; for descriptions of other stalk regions, see also Beavil et al., Proc. Nat'l. Acad. Sci. USA 89:153, 1992; and Figdor et al., Nat. Rev. Immunol. 2:11, 2002). These type II C-lectin or CD molecules may also have junction amino acids (described below) between the stalk region and the transmembrane region or the CTLD. In another example, the 233 amino acid human NKG2A protein (GenBank Accession No. P26715.1) has a hydrophobic portion (transmembrane domain) ranging from amino acids 71-93 and an extracellular domain ranging from amino acids 94-233. The CTLD includes amino acids 119-231 and the stalk region includes amino acids 99-116, which may be flanked by additional junction amino acids. Other type II C-lectin or CD molecules, as well as their extracellular ligand-binding domains, stalk regions, and CTLDs are known in the art (see, e.g., GenBank Accession Nos. NP 001993.2; AAH07037.1; NP 001773.1; AAL65234.1; CAA04925.1; for the sequences of human CD23, CD69, CD72, NKG2A, and NKG2D and their descriptions, respectively).

Linkers may also provide flexibility and room for conformational movement between different components of a recombinant receptor. Commonly used flexible linkers include linker sequence with the amino acids glycine and serine (Gly-Ser linkers). In particular embodiments, the linker sequence includes sets of glycine and serine repeats such as from one to ten repeats of (GlyₓSer_{y})ₙ, wherein x and y are independently an integer from 0 to 10 provided that x and y are not both 0 and wherein n is an integer of 1, 2, 3, 4, 5, 6, 7, 8, 9 or 10). Particular examples include (Gly₄Ser)ₙ (SEQ ID NO: 53), (Gly₃Ser)ₙ(Gly₄Ser)₄ (SEQ ID NO: 54), (Gly₃Ser)ₙ(Gly₂Ser)ₙ (SEQ ID NO: 55), or (Gly₃Ser)₄(Gly₄Ser)₁ (SEQ ID NO: 56). In particular embodiments, the linker is (Gly₄Ser)₄ (SEQ ID NO: 57), (Gly₄Ser)₃ (SEQ ID NO: 58), (Gly₄Ser)₂ (SEQ ID NO: 59), (Gly₄Ser)₁ (SEQ ID NO: 60), (Gly₃Ser)₂ (SEQ ID NO: 61), (Gly₃Ser)₁ (SEQ ID NO: 62), (Gly₂Ser)₂ (SEQ ID NO: 63) or (Gly₂Ser)₁, GGSGGGSGGSG (SEQ ID NO: 64), GGSGGGSGSG (SEQ ID NO: 65), or GGSGGGSG (SEQ ID NO: 66).

Junction amino acids can be used to connect components of a recombinant receptor when the distance, space, or flexibility provided by a spacer or linker is not needed and/or wanted. In particular embodiments, junction amino acids are 9 amino acids or less (e.g., 2, 3, 4, 5, 6, 7, 8, or 9 amino acids). In particular embodiments, a glycine-serine doublet can be used as a suitable junction amino acid linker. In particular embodiments, a single amino acid, e.g., an alanine or a glycine, can be used as a junction amino acid.

In particular embodiments, the recombinant receptor is a CAR. In particular embodiments, the CAR includes an anti-CD19 CAR or an anti-CD33 CAR. In particular embodiments, an anti-CD19 CAR includes an anti-huCD19 scFV (e.g., G01S), an IgG4 hinge, a CD28 transmembrane domain, a 4-1BB intracellular signaling domain and a CD3ζ intracellular signaling domain. In particular embodiments, an anti-CD19 CAR includes an anti-muCD19 scFV (e.g., FMC63), an IgG4 hinge, a CD28 transmembrane domain, a 4-1BB intracellular signaling domain and a CD3ζ intracellular signaling domain. In particular embodiments, an anti-CD33 CAR includes an anti-CD33 scFV (e.g., h2H12), an IgG4 hinge, a CD28 transmembrane domain, a 4-1BB intracellular signaling domain and a CD3ζ intracellular signaling domain.

In particular embodiments, the recombinant receptor is an orthogonal CAR. In particular embodiments, an orthogonal CAR is a CAR with nonstandard amino acids.

### (iii) Recombinant Receptor Encoding Genetic Constructs and Methods For Genetically Modifying Cells

Recombinant receptor-encoding genetic constructs (or genetic constructs) encode a recombinant receptor. In particular embodiments, a gene encoding a recombinant receptor can be introduced into cells as part of a vector. A “vector” is a nucleic acid molecule that is capable of transporting another nucleic acid. Vectors may be, e.g., plasmids (DNA plasmids or RNA plasmids), transposons, cosmids, bacterial artificial chromosomes, viruses, or phage. An “expression vector” is a vector that is capable of directing the expression of a protein encoded by one or more genes carried by the vector when it is present in the appropriate environment. In particular embodiments, the recombinant receptor-encoding construct can be in the form of plasmid DNA. In particular embodiments, all components required to express a recombinant receptor and other components of the method can be expressed by a single vector. In particular embodiments, all components required to express a recombinant receptor and other components of the method can be split across two or more vectors. In particular embodiments, the vector can be a self-inactivating vector. In particular embodiments, a self-inactivating (SIN) vector is a vector including a deletion within the 3′ long terminal repeat (LTR).

Beyond encoding a recombinant receptor, recombinant receptor-encoding constructs can include or encode numerous other components, such as regulatory components, transposable elements, selection cassettes, transduction markers, reporters, enhancing transgenes, or other useful components (see, for example, FIG. 5).

In particular embodiments, the recombinant receptor-encoding construct includes regulatory components. Regulatory components can include promoters, enhancers, insulators, and other sequences that regulate gene expression.

Promoters can include general promoters, tissue-specific promoters, cell-specific promoters, and/or promoters specific for the cytoplasm. Promoters may include strong promoters, weak promoters, constitutive expression promoters, and/or inducible promoters. Constitutive promoters are promoters that allow for the continual transcription of its associated gene or genes. Inducible promoters direct expression in response to certain conditions, signals or cellular events. For example, the promoter may be an inducible promoter that requires a particular ligand, small molecule, transcription factor or hormone protein in order to effect transcription from the promoter.

Particular examples of constitutive promoters include human elongation factor Iα promoter (EFIα, including EFIα(s) and EFIα (L)), myeloproliferative sarcoma virus (MND), cytomegalovirus (CMV), simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as the actin promoter, the myosin promoter, the hemoglobin promoter, and the creatine kinase promoter.

The promoter can alternatively be an inducible promoter. Examples of inducible promoters include an inducible synthetic promoter (iSynPro), a metallothionine promoter, a glucocorticoid promoter, a progesterone promoter, and a tetracycline promoter.

Additional regulatory elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. Examples of enhancers include SV40 early gene enhancer, enhancers from human actin, myosin, hemoglobin, muscle creatine kinase, and those derived from polyoma vims, human or murine cytomegalovirus (CMV), the long-term repeat from various retroviruses such as murine leukemia vims, murine or Rous sarcoma vims, EBV, and HIV. See, Enhancers and Eukaryotic Expression, Cold Spring Harbor Press, Cold Spring Harbor, N.Y. 1983 for additional enhancers.

In particular embodiments, an insulator flanks each side of the transgene to reduce the potential for silencing. An insulator is a cis-regulatory element that has enhancer-blocking or barrier function. Enhancer-blocker insulators block enhancers from acting on the promoter of nearby genes. Barrier insulators prevent euchromatin silencing. Examples of insulators include the chicken hypersensitivity site 4 insulator (cHS4), CTCF insulator, HS5 insulator, gypsy insulator, b-globin locus insulator, Kaiso insulator, A1 insulator, and A2 insulator.

Particular embodiments disclosed herein utilize transposable elements to deliver large transgenes such as recombinant receptor-encoding constructs. In particular embodiments, the efficiency of integration, the size of the DNA sequence that can be integrated, and the number of copies of a DNA sequence that can be integrated into a genome can be improved by using transposable elements. Transposable elements include transposons and transposases. A transposon refers to a discrete DNA fragment containing a transposon gene. The flanking sequences are terminal inverted repeats (TIRs) containing transposase-binding sites. Transposases bind the TIR and catalyze the movement of the transposon to another portion of the genome. Particular embodiments include a vector carrying the recombinant receptor-encoding construct including a transposon and a vector carrying the transposase. In particular embodiments, the vector carrying the recombinant receptor-encoding construct including a transposon is a plasmid. In particular embodiments, the vector carrying the transposase is a plasmid.

The piggyBac™ (PB) transposase (Poseida Therapeutics, San Diego, CA) is a compact functional transposase protein that is described in, for example, Fraser et al., Insect Mol. Biol., 5:141-51, 1996; Mitra et al., EMBO J. 27:1097-1109, 2008; Ding et al., Cell, 122:473-83, 2005; and U.S. Pat. Nos. 6,218, 185; 6,551,825; 6,962,810; 7,105,343; and 7,932,088. Hyperactive piggyBac™ transposases are described in U.S. Pat. No. 10, 131,885.

In addition to PB, a number of transposases have been described in the art that facilitate insertion of nucleic acids into a genome. Examples of such transposases include Sleeping Beauty (e.g., derived from salmonid fish); hyperactive Sleeping Beauty (SB100X); mariner (e.g., derived from Drosophila); frog prince (e.g., derived from Rana pipiens); Tol1; Tol2 (e.g., derived from medaka fish); TcBuster™ (e.g., derived from the red flour beetle Tribolium castaneum), Helraiser, Himar1, Passport, Minos, Ac/Ds, PIF, Harbinger, Harbinger3-DR, HSmar1, and spinON.

In particular embodiments, PB transposase has the sequence as set forth in GenBank ABS12111.1. In particular embodiments, the SB has the sequence as set forth in PDB: 5UNK_A. In particular embodiments, SB100X has the sequence as set forth in PDB: 5CR4_A and PDB: 5CR4_B. In particular embodiments, a Frog Prince transposase has the sequence as set forth in GenBank: AAP49009.1. See also US2005/0241007. In particular embodiments, a TcBuster transposase has the sequence as set forth in GenBank: ABF20545.1. In particular embodiments, a Tol2 transposase has the sequence set forth in GenBank: BAA87039.1. Additional information on DNA transposons can be found, for instance, in Muñoz-López & García Pérez, Curr Genomics, 11(2):115-128, 2010.

In particular embodiments, a selection cassette provides for positive selection or negative selection of a desired cell population. Negative selection is when several cell types are removed, leaving the cell type of interest. Positive selection involves targeting the desired cell population to only retain desired cells.

A selection cassette can encode proteins that (a) confer resistance to antibiotics or other toxins, (b) complement auxotrophic deficiencies, or (c) supply critical nutrients not available from complex media, e.g., the gene encoding D-alanine racemase for Bacilli. Any number of selection systems may be used to recover transformed cells. In particular embodiments, a positive selection cassette includes resistance genes to neomycin, hygromycin, ampicillin, puromycin, phleomycin, zeomycin, blasticidin, or viomycin. In particular embodiments, a selection cassette includes the DHFR (dihydrofolate reductase) gene or DHFR double mutant (DHFRdm) gene providing resistance to methotrexate (MTX), the MGMT P140K gene responsible for the resistance to O⁶BG/BCNU, the HPRT (Hypoxanthine phosphoribosyl transferase) gene responsible for the transformation of specific bases present in the HAT selection medium (aminopterin, hypoxanthine, thymidine) or other genes for detoxification with respect to some drugs. In particular embodiments, the selection agent includes neomycin, hygromycin, puromycin, phleomycin, zeomycin, blasticidin, viomycin, ampicillin, O⁶BG/BCNU, MTX, tetracycline, aminopterin, hypoxanthine, thymidine kinase, DHFR, Gln synthetase, or ADA.

In particular embodiments, the selection cassette includes DHFRdm and the selection agent includes MTX. In particular embodiments, the method does not require a selection cassette to acquire highly purified cell populations.

In particular embodiments, negative selection cassettes include a gene for transformation of a substrate present in the culture medium into a toxic substance for the cell that expresses the gene. These molecules include detoxification genes of diptheria toxin (DTA) (Yagi et al., Anal Biochem. 214(1):77-86, 1993; Yanagawa et al., Transgenic Res. 8(3):215-221, 1999), the kinase thymidine gene of the Herpes virus (HSV TK) sensitive to the presence of ganciclovir or FIAU. The HPRT gene may also be used as a negative selection by addition of 6-thioguanine (6TG) into the medium. and for all positive and negative selections, a poly A transcription termination sequence from different origins, the most classical being derived from SV40 poly A, or a eukaryotic gene poly A (bovine growth hormone, rabbit β-globin, etc.).

Transduction markers may be selected from at least one of a truncated human EGFR (EGFR; see Wang et al., Blood 118:1255, 2011); a truncated human epidermal growth factor receptor 2 (Her2tG); a truncated CD19 (tCD19; see Budde et al., Blood 122:1660, 2013); an extracellular domain of human CD34; and/or RQR8 which combines target epitopes from CD34 (see Fehse et al, Mol. Therapy 1(5 Pt 1); 448-456, 2000) and CD20 antigens (see Philip et al, Blood 124:1277-1278).

In particular embodiments, reporters include sequences encoding fluorescent markers or bioluminescence markers. In particular embodiments, reporting elements include fusion genes wherein the fusion gene includes a fluorescent marker and a bioluminescence marker.

Fluorescent markers can be useful in identifying, tracking, isolating, or assessing the activity of modified cells. Fluorescent markers include fluorescent proteins including blue fluorescent proteins (e.g. eBFP, eBFP2, Azurite, mKalama1, GFPuv, Sapphire, T-sapphire); cyan fluorescent proteins (e.g. eCFP, Cerulean, CyPet, AmCyanI, Midoriishi-Cyan, mTurquoise); green fluorescent proteins (e.g. GFP, GFP-2, tagGFP, turboGFP, EGFP, Emerald, Azami Green, Monomeric Azami Green (mAzamigreen)), CopGFP, AceGFP, avGFP, ZsGreenI, Oregon Green™ (Thermo Fisher Scientific)); Luciferase; orange fluorescent proteins (mOrange, mKO, Kusabira-Orange, Monomeric Kusabira-Orange, mTangerine, tdTomato); red fluorescent proteins (mKate, mKate2, mPlum, DsRed monomer, mCherry, mRuby, mRFP1, DsRed-Express, DsRed2, DsRed-Monomer, HcRed-Tandem, HcRedI, AsRed2, eqFP611, mRaspberry, mStrawberry, Jred, Texas Red™ (Thermo Fisher Scientific)); far red fluorescent proteins (e.g., mPlum and mNeptune); yellow fluorescent proteins (e.g., YFP, eYFP, Citrine, SYFP2, Venus, YPet, PhiYFP, ZsYellowI); and tandem conjugates.

Bioluminescent markers refer to reporters that cause chemiluminescence using an enzyme-catalyzed process in a living organism. Bioluminescent markers include firefly luciferase (ffluc), Renilla luciferase, Click beetle luciferase, Railroad worm luciferase, Rluc8 (mutant of Renilla luciferase), Gaussia luciferase, Gaussia-Dura luciferase, Cypridina luciferase, Metridia luciferase, and Oluc.

In particular embodiments, a reporter can be encoded by a fusion gene encoding a bioluminescent marker and a fluorescent marker (e.g., GFP: ffLuc).

An enhancing transgene is a gene that facilitates better cytotoxicity by a recombinant receptor. In particular embodiments, the enhancing transgene is expressed when a recombinant receptor is active. In particular embodiments the enhancing transgene includes PD1 (A99L): MYD88. Additional examples of enhancing transgenes include PD1 (A99L): CD28, PD1 (A99L): CD2, caSTAT5a, CCR2, dnSHP1, caBCL2, dnFADD, NOTCH1Ic, cFLIP.v3, dnSHP2, caHIF1a, dnDGKa, dnBLIMP1, dnTGFbRII, caAKT, dnNRDP1, and PCK1.

A cell can be genetically modified to express a recombinant receptor-encoding construct according to the present disclosure by any methods known in the art.

In particular embodiments, a recombinant receptor-encoding construct is produced using recombinant DNA techniques. A nucleic acid encoding the several regions of the recombinant receptor can be prepared and assembled into a complete coding sequence by standard techniques of molecular cloning. The resulting coding regions can be inserted into an expression vector and used to transform a cell or a population of cells.

In particular embodiments, a gene encoding a recombinant receptor can be introduced into cells using electroporation. Electroporation has been used in both in vitro and in vivo procedures to introduce foreign material into living cells. With in vitro applications, a sample of live cells is generally first mixed with an agent of interest (e.g., a genetic construct) to be incorporated into the cell and placed between electrodes such as parallel plates. Then, the electrodes apply an electrical field to the cell/agent of interest mixture. Examples of systems that perform in vitro electroporation include the 4D-Nucleofector™ System (Lonza, Basel, Switzerland), Electro Cell Manipulator ECM600 and the Electro Square Porator T820, both made by the BTX Division of Genetronics, Inc (see U.S. Pat. No. 5,869,326), and Gene Pulser Xcell Electroporation System (Bio-RAD, Hercules).

Electroporation techniques (both in vitro and in vivo) function by applying a brief high voltage pulse to electrodes positioned around the electroporation region. The electric field generated between electrodes causes the cell membranes to temporarily become porous, whereupon molecules the construct of genetic enter the cells. In known electroporation applications, this electric field can include a single square wave pulse on the order of 1000 V/cm, of 100 μs duration.

In certain examples, the electric field has a strength of from 1 V/cm to 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, or 10 kV/cm. In certain examples from 0.5 kV/cm to 4.0 kV/cm is used under in vitro conditions. However, the electric field strengths may be lowered where the number of pulses delivered to the target site are increased. Thus, pulsatile delivery of electric fields at lower field strengths can be used.

In certain examples, the application of the electric field is in the form of multiple pulses such as double pulses of the same strength and capacitance or sequential pulses of varying strength and/or capacitance. “Pulse” includes one or more electric pulses at variable capacitance and voltage and including exponential and/or square wave and/or modulated wave/square wave forms.

In certain examples, the electric pulse is delivered as a waveform selected from an exponential wave form, a square wave form, a modulated wave form and a modulated square wave form.

Certain examples include use of an electric field applied to cells at a field strength of between 1 V/cm and 10 kV/cm, for a period of 100 milliseconds or more (e.g., 15 minutes or more).

Certain examples may utilize targeted genetic engineering approaches. The CRISPR (Clustered Regularly Interspaced Short Palindromic Repeats)/Cas (CRISPR-associated protein) nuclease system is an engineered nuclease system used for genetic engineering that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Particular embodiments utilize zinc finger nucleases (ZFNs) as gene editing agents. ZFNs are a class of site-specific nucleases engineered to bind and cleave DNA at specific positions. For additional information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; No. 7,585,849; 7,595,376; 6,903,185; 6,479,626; US 2003/0232410 and US 2009/0203140 as well as Gaj et al., Nat Methods, 2012, 9(8):805-7; Ramirez et al., Nucl Acids Res, 2012, 40(12):5560-8; Kim et al., Genome Res, 2012, 22(7):1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. TALENs refer to fusion proteins including a transcription activator-like effector (TALE) DNA binding protein and a DNA cleavage domain. For additional information regarding TALENs, see U.S. Pat. Nos. 8,440,431; 8,440,432; 8,450,471; 8,586,363; and 8,697,853; as well as Joung and Sander, Nat Rev Mol Cell Biol, 2013, 14(I):49-55; Beurdeley et al., Nat Commun, 2013, 4:1762; Scharenberg et al., Curr Gene Ther, 2013, 13(4):291-303; Gaj et al., Nat Methods, 2012, 9(8):805-7; Miller, et al. Nature biotechnology 29, 143-148 (2011); Christian, et al. Genetics 186, 757-761 (2010); Boch, et al. Science 326, 1509-1512 (2009); and Moscou, & Bogdanove, Science 326, 1501 (2009).

Cells that have been successfully genetically modified to express a recombinant receptor ex vivo can be sorted based on, for example, expression of a transduction marker, and further processed.

Methods disclosed herein can also use viral vectors to genetically modify cells to express a recombinant receptor-encoding construct.

Viral vectors can be derived from numerous viruses. “Lentivirus” refers to a genus of retroviruses that are capable of infecting dividing and non-dividing cells and typically produce high viral titers. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

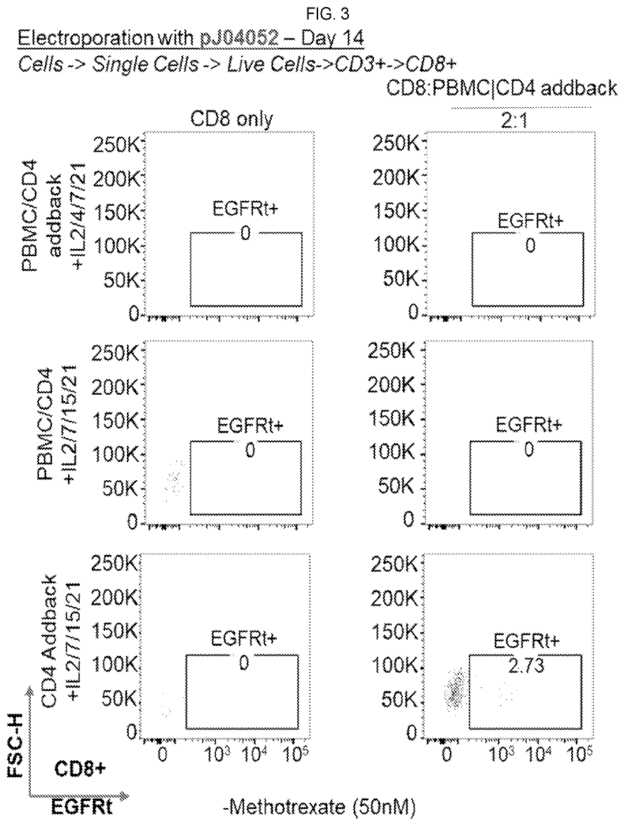

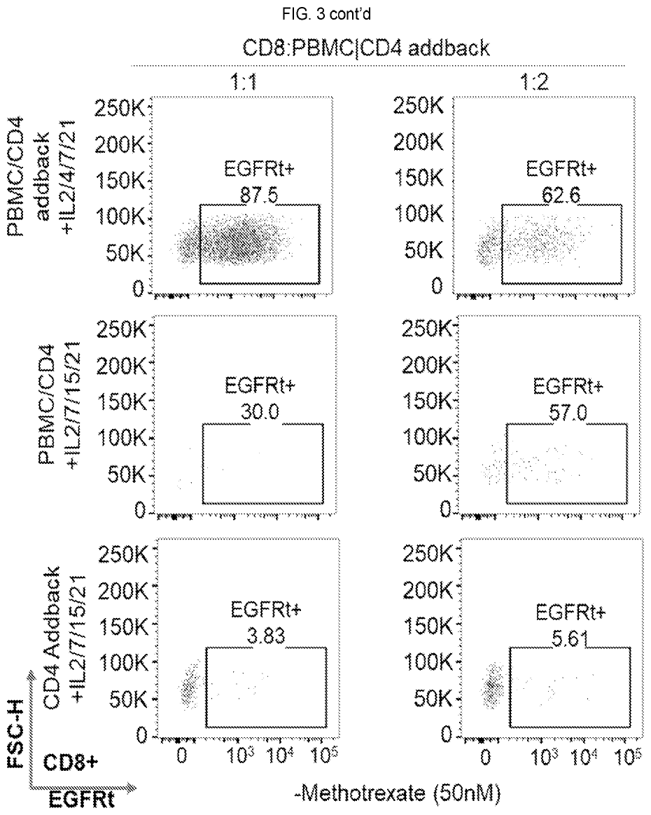

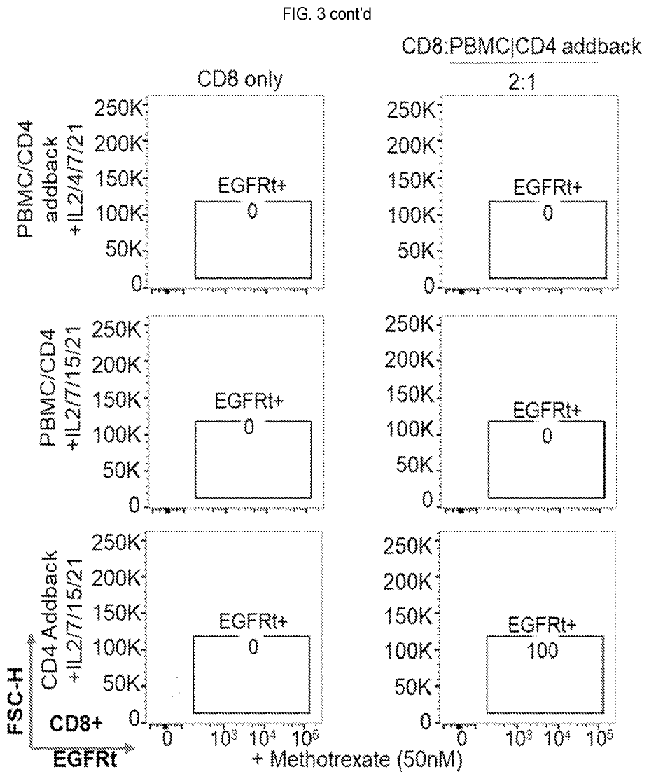

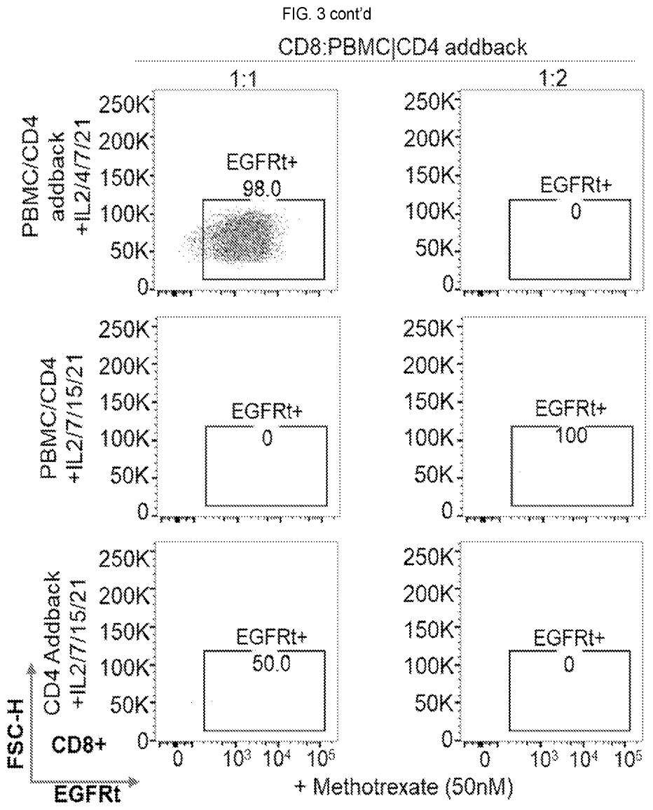

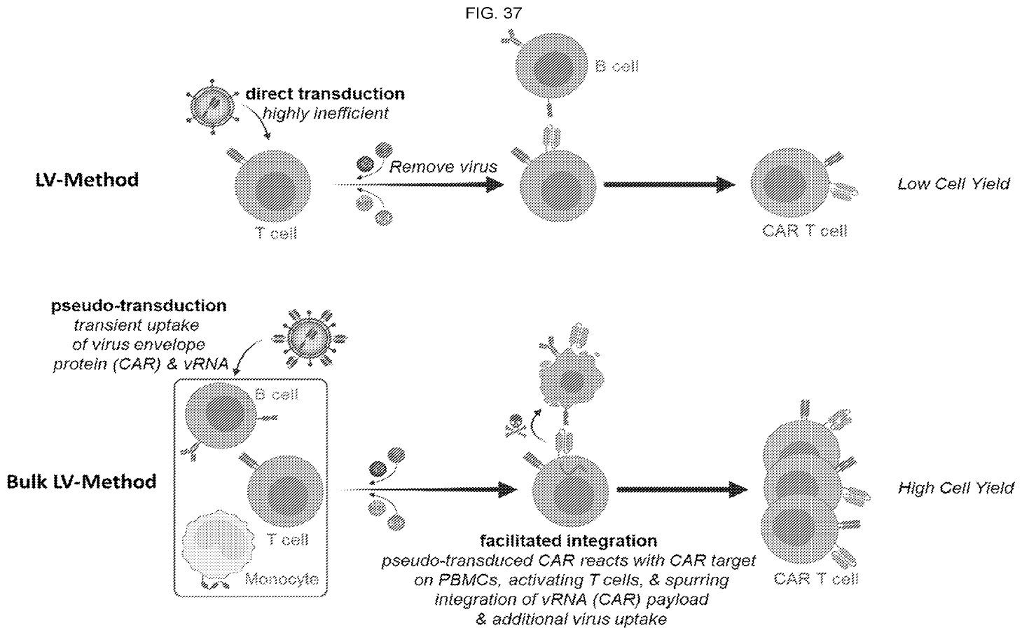

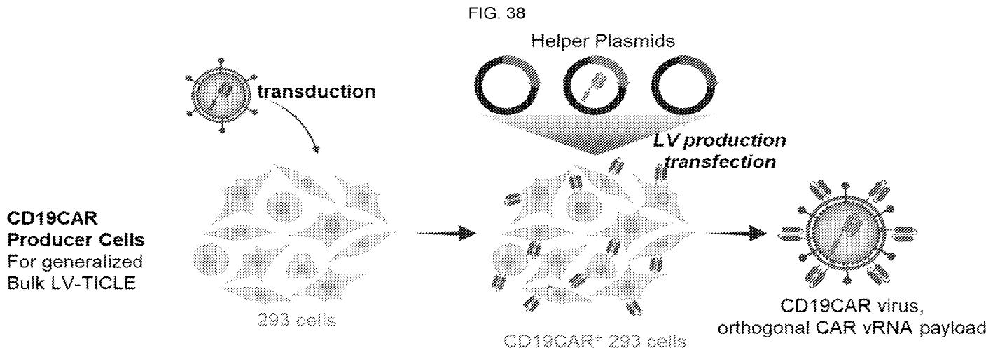

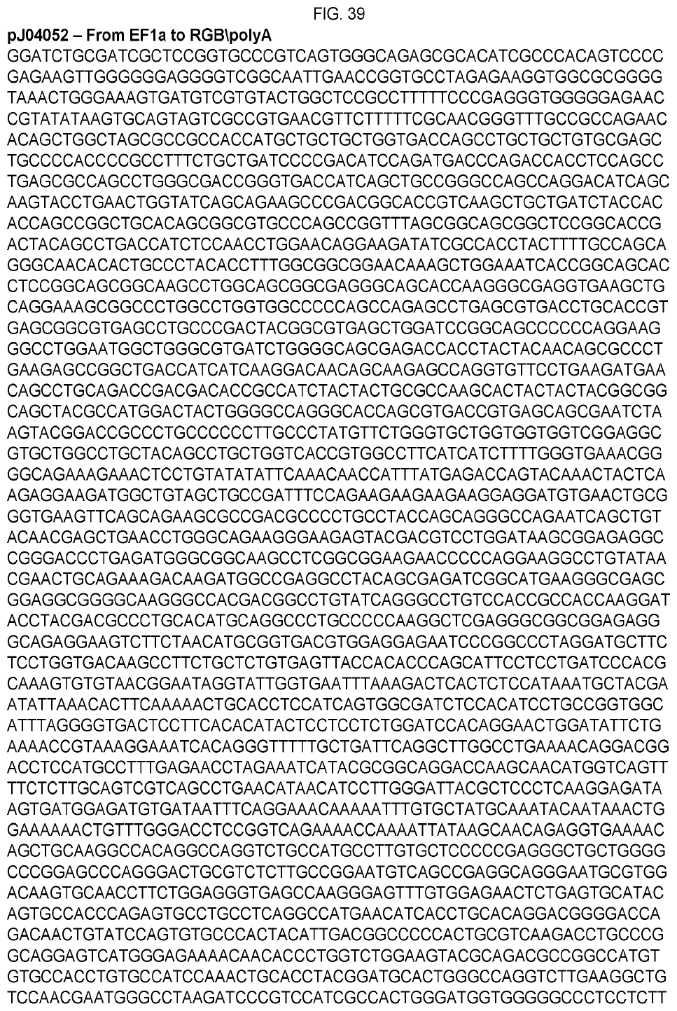

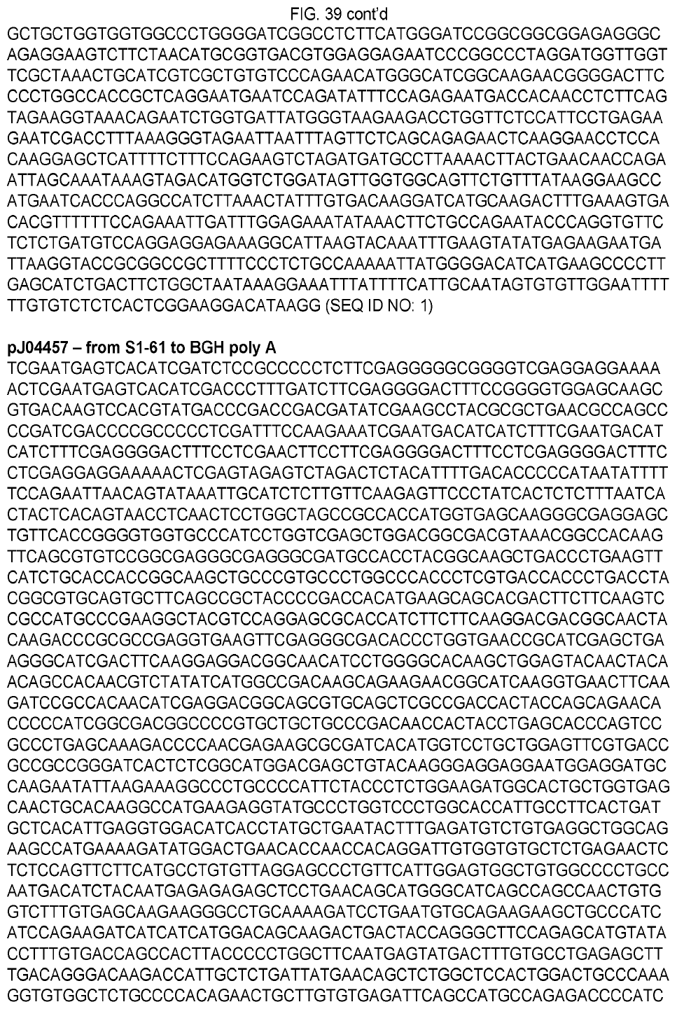

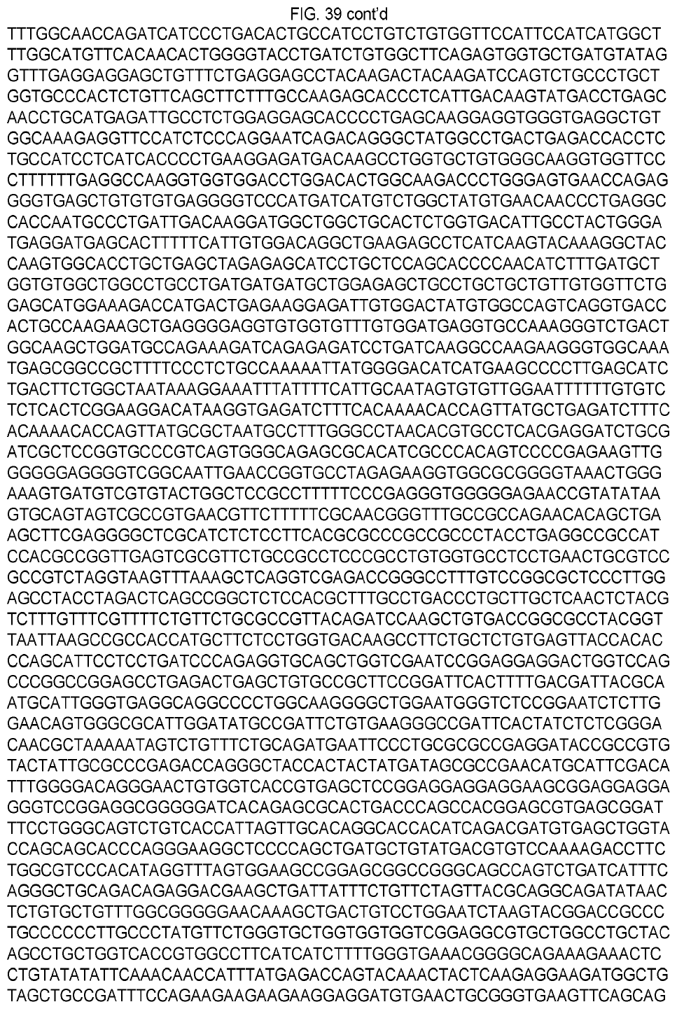

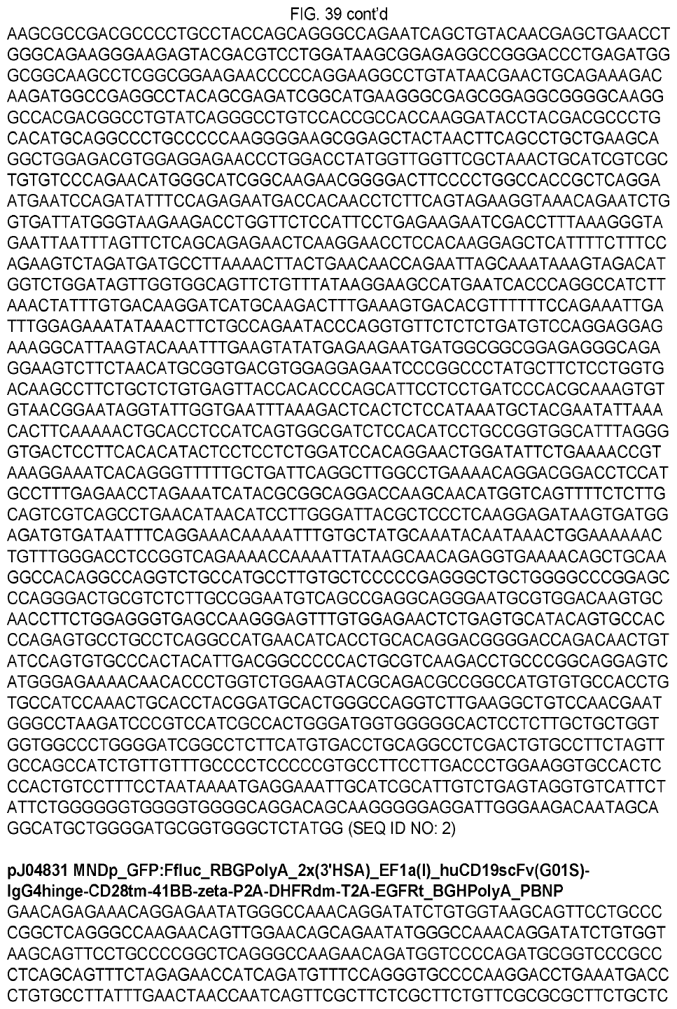

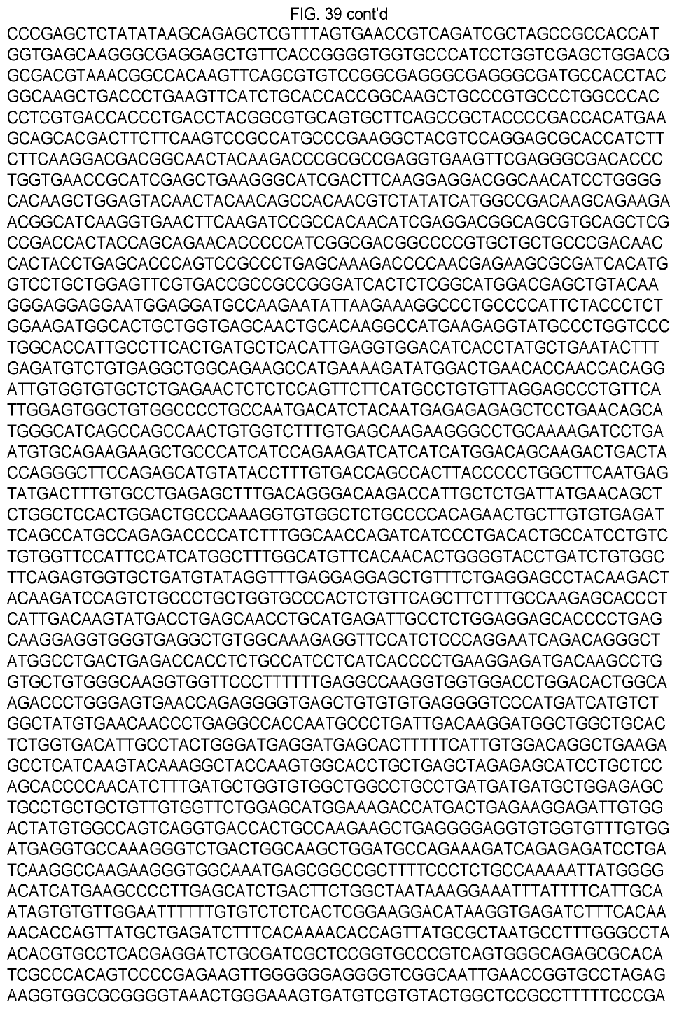

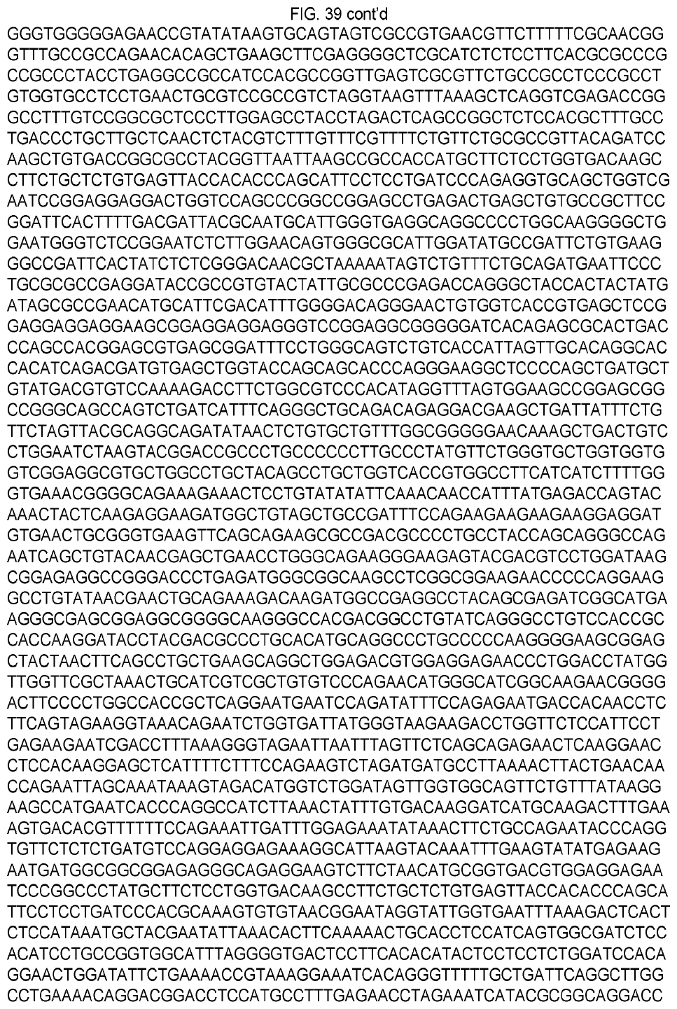

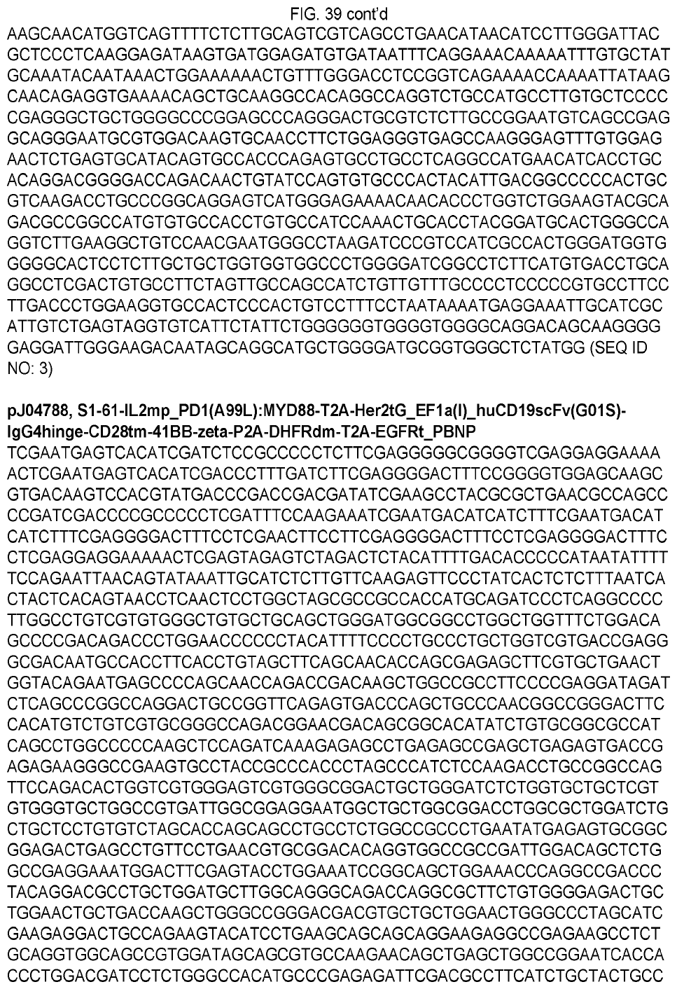

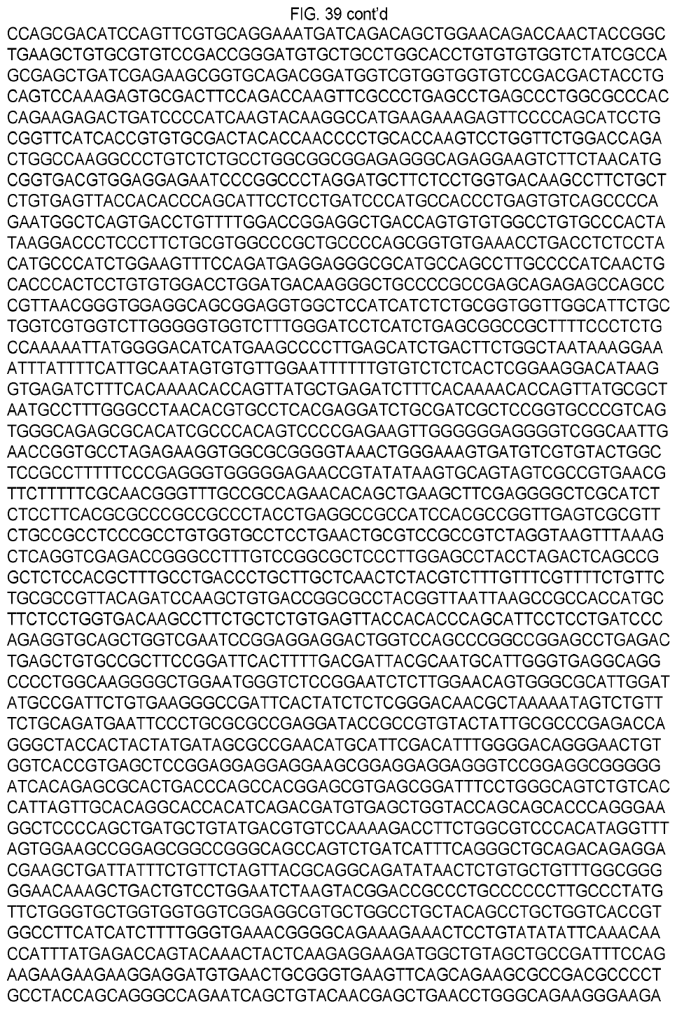

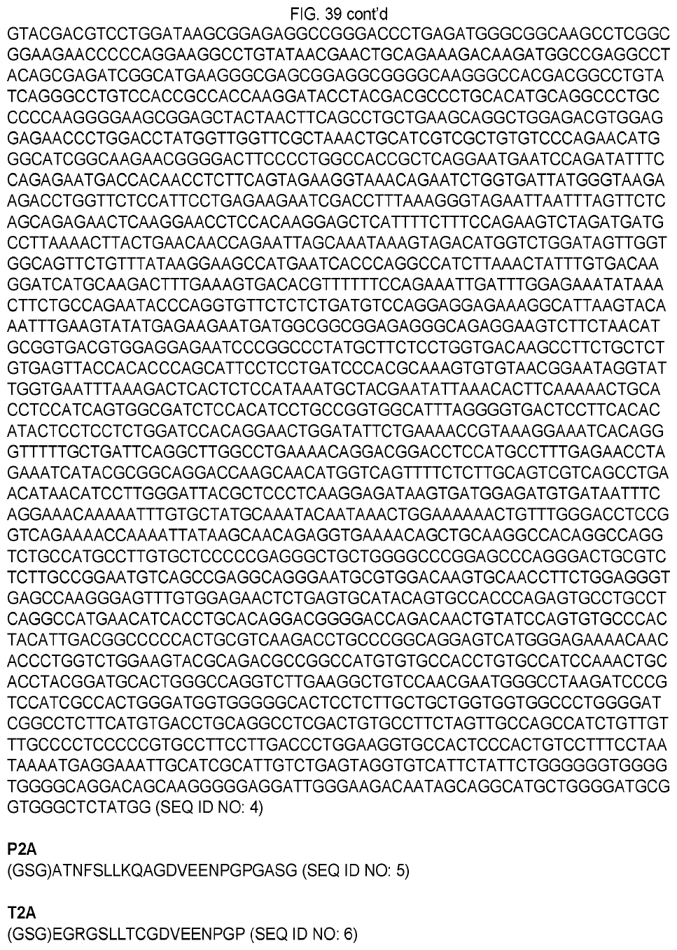

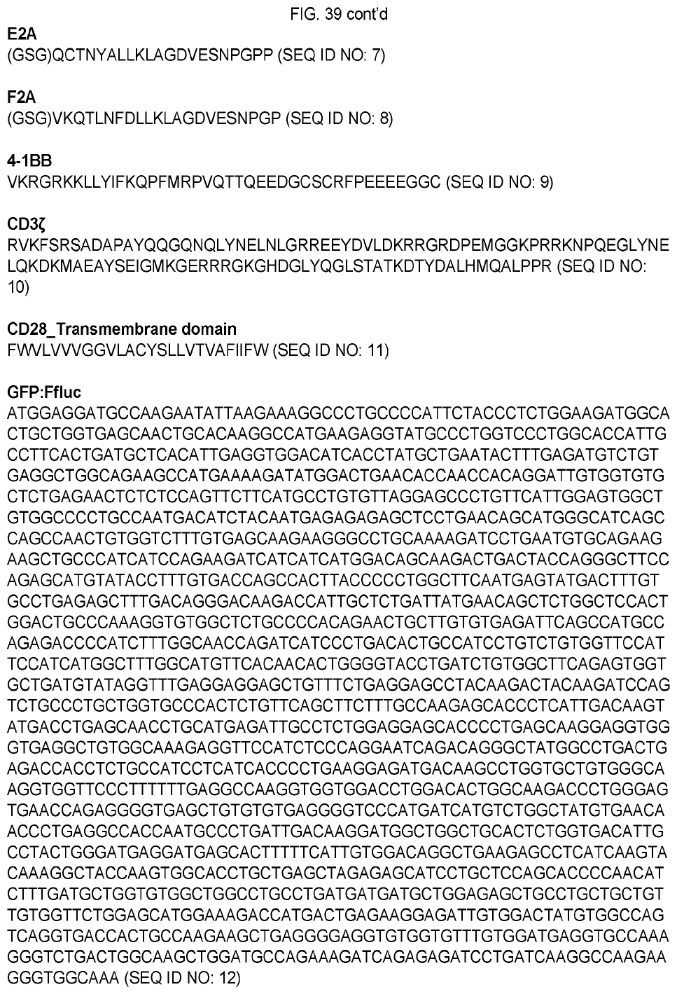

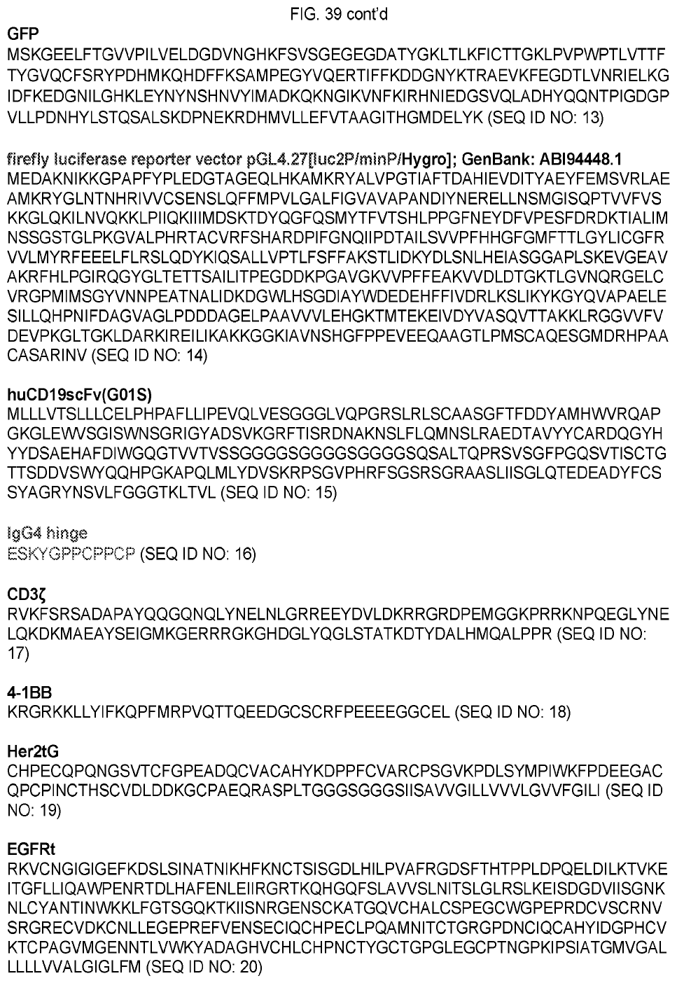

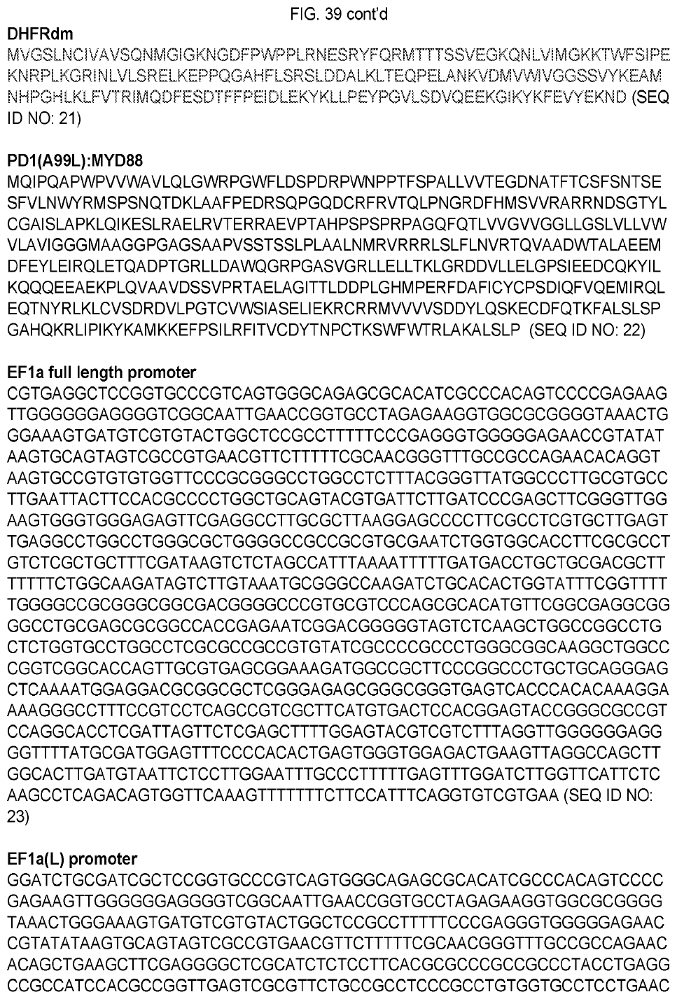

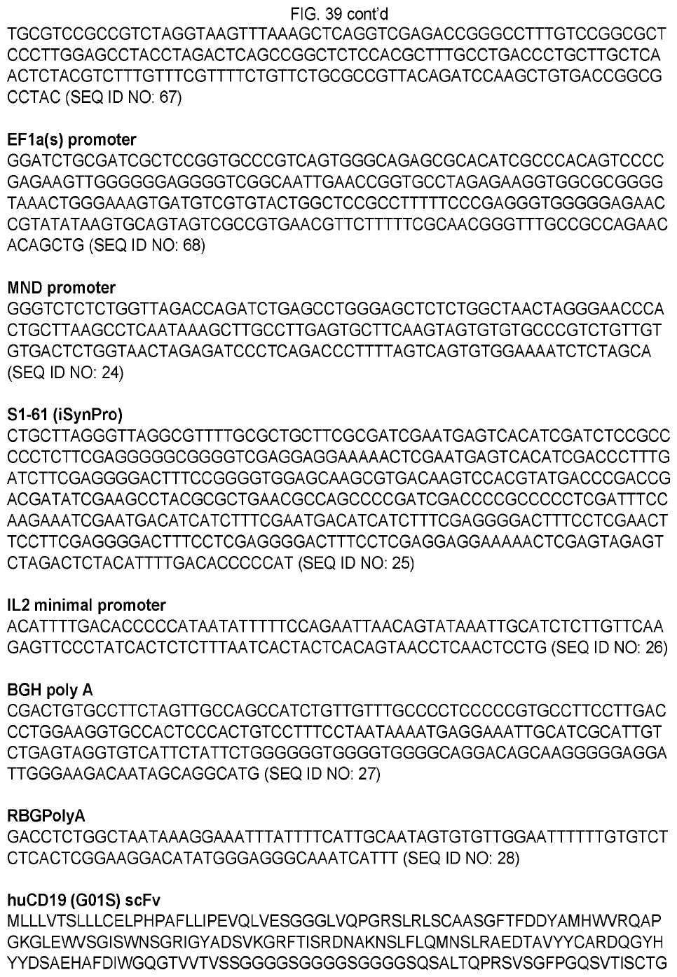

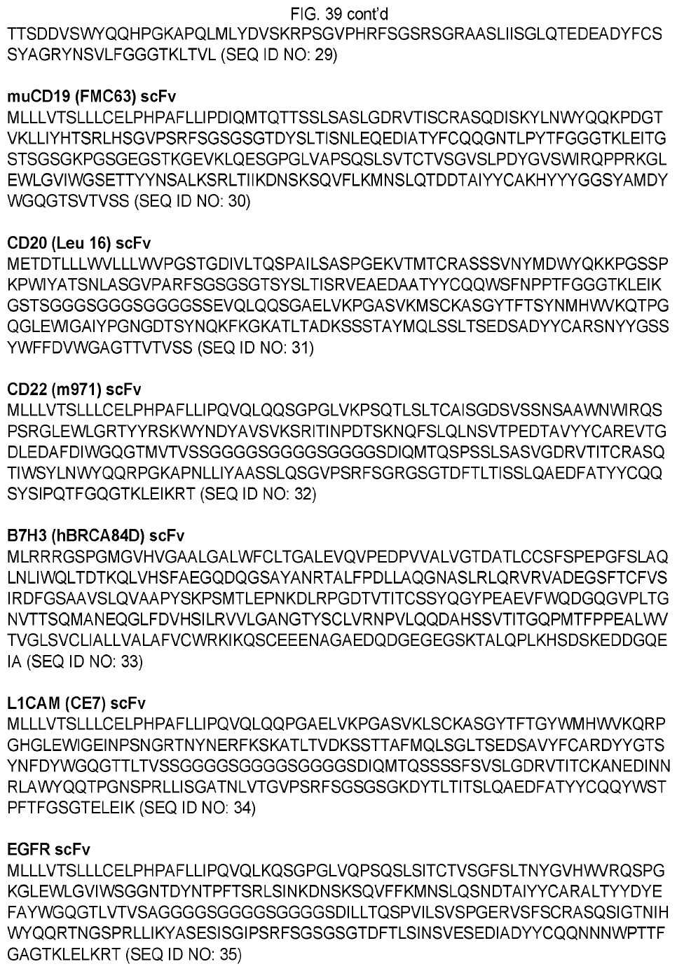

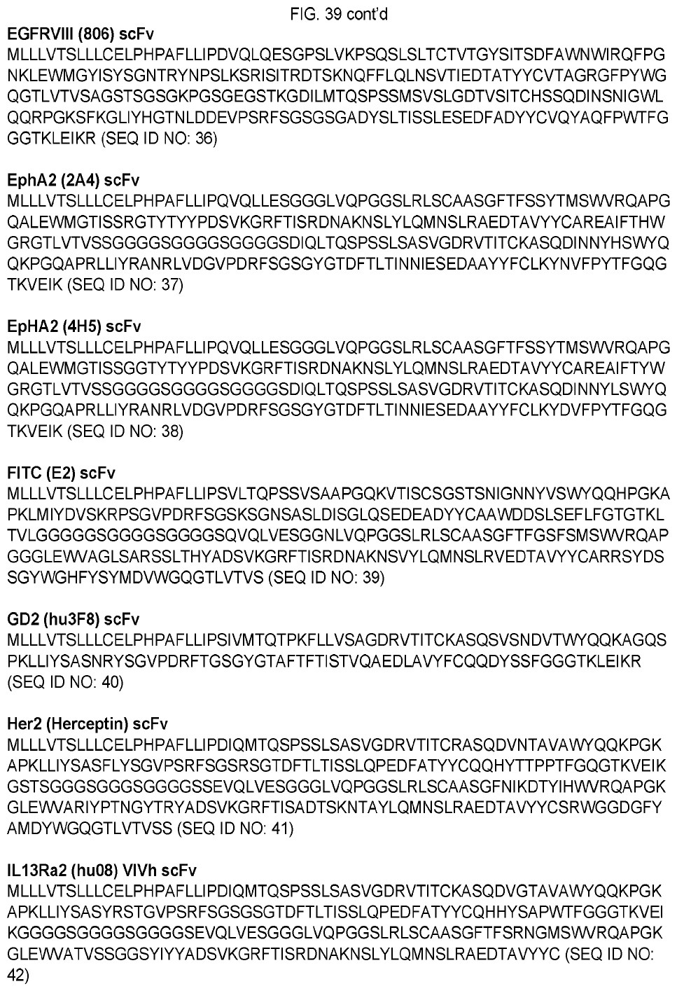

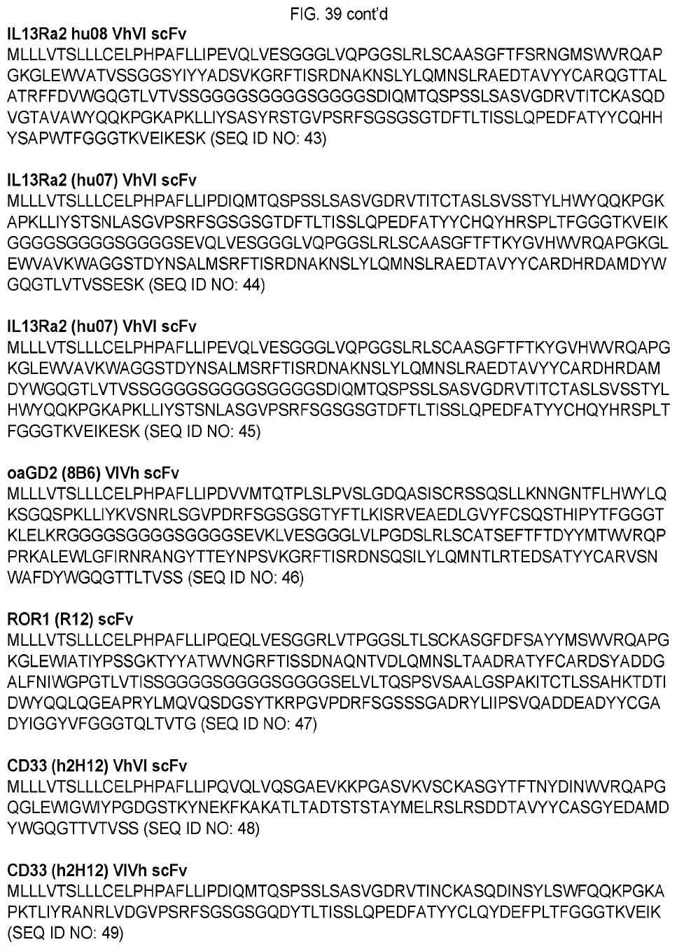

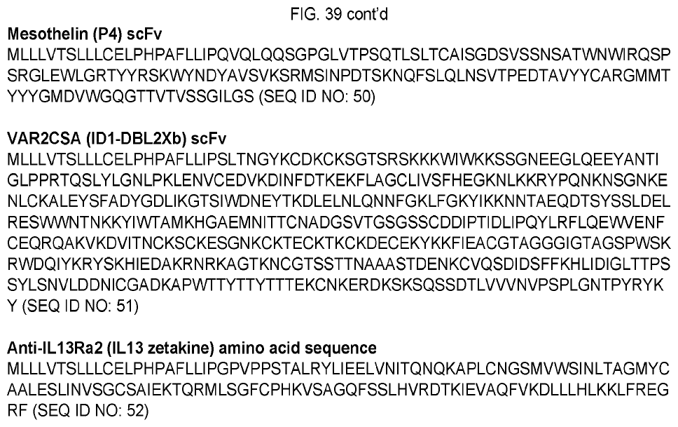

Additional examples of viral vectors include those derived from foamy viruses, adenoviruses (e.g., adenovirus 5 (Ad5), adenovirus 35 (Ad35), adenovirus 11 (Ad11), adenovirus 26 (Ad26), adenovirus 48 (Ad48) or adenovirus 50 (Ad50)), adeno-associated virus (AAV; see, e.g., U.S. Pat. No. 5,604,090; Kay et al., 2000; Nakai et al., z1998), alphaviruses, cytomegaloviruses (CMV), flaviviruses, herpes viruses (e.g., herpes simplex), influenza viruses, papilloma viruses (e.g., human and bovine papilloma virus; see, e.g., U.S. Pat. No. 5,719,054), poxviruses, vaccinia viruses, etc. See Kozarsky and Wilson, 1993; Rosenfeld, et al., 1991; Rosenfeld, et al., 1992; Mastrangeli, et al., 1993; Walsh, et al., 1993; and Lundstrom, 1999. Examples include modified vaccinia Ankara (MVA) and NYVAC, or strains derived therefrom. Other examples include avipox vectors, such as a fowlpox vectors (e.g., FP9) or canarypox vectors (e.g., ALVAC and strains derived therefrom). For additional information regarding viral vectors for gene delivery, see Kozarsky and Wilson, 1993, Current Opinion in Genetics and Development 3:499-503, Rosenfeld, et al., 1991, Science 252:431-434; Rosenfeld, et al., 1992, Cell 68:143-155; Mastrangeli, et al., 1993, J. Clin. Invest. 91:225-234; Walsh, et al., 1993, Proc. Soc. Exp. Bioi. Med. 204:289-300; and Lundstrom, 1999, J. Recept. Signal Transduct. Res. 19:673-686; Miller, et al., 1993, Meth. Enzymol. 217:581-599); Naldini et al. (1996) Science 272(5259):263-267; Naldini et al. (1996) Proceedings of the National Academy of Sciences 93(21):11382-11388; Zufferey et al. (1997) Nature biotechnology 15(9):871-875; Dull et al. (1998) Journal of virology 72(11):8463-8471; U.S. Pat. Nos. 6,013,516; and 5,994,136). Methods of using viral vectors are shown in FIGS. 3, 37, and 38.

### (iv) Culture Conditions

Standard culture conditions for culturing T cells are well known in the art. The current disclosure provides co-culturing genetically modified T cells with an autologous negative fraction of a cell isolation procedure. Herein, recombinant receptor-expressing T cells refer to the T cells that were isolated and genetically modified with a recombinant receptor-encoding construct to express a recombinant receptor. The recombinant receptor-expressing T cells are different from feeder cells which can include T cells. Feeder cells are cells that are cultured with the recombinant receptor-expressing T cells to provide stimulation, growth factors, and/or nutrients to the recombinant receptor-expressing T cells. In particular embodiments, recombinant receptor-expressing T cells include CD8+ and/or CD4+ T cells.

Culture conditions include culture vessel, temperature, O₂ level, CO₂ level, humidity, culture time, culture media, and activation/expansion agents (e.g., nutrients, amino acids, antibiotics, ions, and/or stimulatory factors, such as cytokines, chemokines, antigens, binding partners, fusion proteins, recombinant soluble receptors, and feeder cells). In particular embodiments, culturing recombinant receptor-expressing T cells includes incubating recombinant receptor-expressing T cells within a culture media in a culture vessel, wherein the culture media and recombinant receptor-expressing T cells are exposed to an atmosphere with a selected temperature, O₂ level, CO₂ level, and humidity for a select culture time.

Cell populations can be incubated in a culture-initiating composition to expand T cell populations. The incubation can be carried out in a culture vessel, such as a bag, cell culture plate, flask, chamber, chromatography column, cross-linked gel, cross-linked polymer, column, culture dish, hollow fiber, microtiter plate, silica-coated glass plate, tube, tubing set, well, vial, or other container for culture or cultivating cells.

Cells can be cultured in a gas-permeable culture vessel. Gas permeable culture vessels may be used to culture cells where the cells rest upon a gas permeable surface. In many instances, the gas permeable membrane is non-porous, liquid impermeable, and hydrophobic.

“Gas permeable membrane” is a layer that allows gas to pass through. Gases these membranes may be permeable to include O₂, CO₂, and N₂. Gas permeable silicone membranes 0.005 to 0.007 inches thick may be used (see, for example, U.S. Pat. No. 9,567,565). Further, cell culture devices containing such gas permeable membranes include those in the G-REX® series, which may be obtained from Wilson Wolf Corporation, (Saint Paul, MN) (see, e.g., P/Ns 85500S-CS and 81100S). Other examples of gas permeable membranes and devices containing them are gas permeable plates available from Coy Lab Products (see cat. no. 8602000). These plates allow for the control of O₂ levels that cells are contacted with in incubators. Specifications of exemplary plates are: 25 μm polymer film which allows high gas transfer rate while retaining liquid, O₂ permeability greater than 9000 cm³/M², CO₂ permeability greater than 7000 cm³/M².

The gas permeable material can reside in a horizontal or substantially horizontal position during culture in order for cells to gravitate to the gas permeable material and distribute substantially across the entire surface of the gas permeable material, and, when desired, in a uniform surface density. When the gas permeable membrane is located below the cells being cultured, it will be recognized that the weight of media the gas permeable material can move downward slightly in areas where it is not in direct contact with a support.

Use of a gas permeable cell culture vessel, such as G-REX™ (Wilson-Wolf) dramatically reduces T cell apoptosis during culture, resulting in more efficient expansion in vitro. Gas exchange occurs across a gas permeable silicon membrane at the base of the flask, preventing hypoxia while allowing a greater depth of medium above the cells, providing more nutrients and diluting waste products.

The culture conditions include a temperature suitable for the growth of recombinant receptor-expressing T cells. In particular embodiments, the temperature ranges from 31° C. to 40° C. In particular embodiments, the temperature includes 31° C., 32° C., 33° C., 34° C., 35° C., 36° C., 37° C., 38° C., 39° C., or 40° C. In particular embodiments, the temperature includes 37° C. Skilled practitioners will appreciate that the temperature can be changed at specific time point(s) during culturing, e.g., on an hourly or daily basis.

The culture conditions include an O2 level suitable for the growth of T cells. In particular embodiments, the culture atmosphere contains at least 1% O2, at least 5% O2, at least 10% O2, at least 15% O2, at least 16% O2, at least 17% O2, at least 18% O2, at least 19% O2, or at least 20% O2.

The culture conditions include an CO2 level suitable for the growth of T cells. In particular embodiments, the culture atmosphere contains at most 15% CO2. In particular embodiments, during the culture step, the culture medium including the cells can be exposed to an atmosphere containing at most 15% CO2, at most 14% CO2, at most 13% CO2, at most 12% CO2, at most 11% CO2, at most 10% CO2, at most 9% CO2, at most 8% CO2, at most 7% CO2, at most 6% CO2, at most 5% CO2, at most 4% CO2, at most 3% CO2, at most 2% CO2, or at most 1% CO2.

The culture conditions include a humidity level suitable for the growth of T cells. In particular embodiments, the culture step includes exposing the culture atmosphere contains a humidity of at least 20%, of at least 30%, of at least 40%, of at least 50%, of at least 60%, of at least 70%, of at least 80%, of at least 90%, of at least 95%, or of at least 100%.

The culture conditions include a culture time suitable for the growth of T cells. In particular embodiments, the culture time is 1 hour to five months. In particular embodiments, the culture time is 7 weeks, 6 weeks, 5 weeks, 4 weeks, 3 weeks, 2 weeks, 13 days, 12 days, 11 days, 10 days, 9 days, 8 days, 7 days, 6 days, 5 days, 4 days, 3 days, 2 days, or 1 day.

A culture media is a liquid or gel used to support the growth of cells and microorganisms. The culture media may include amino acids, vitamins, inorganic salts, glucose, and in some cases, serum. The culture medium is important for providing nutrients, maintaining pH and osmolality. The culture media, e.g., complete RPMI (Roswell Park Memorial Institute medium), can be supplemented with L-glutamine, antibiotics, and/or serum (or serum replacements). The uncharacterized nature of blood-derived serum composition and lot-to-lot variation of serum make use of a serum-free media with a serum replacement desirable (Pei et al., Arch Androl. 49(5):331-42, 2003).

In some embodiments, the culture media includes X-VIVO™15 (Lonza, Basel, Switzerland), AIM-V (Life Technologies, Carlsbad, CA), Cellgro SCGM (Mediatech, Manassas, VA), Prime-XV (Irvine Scientific, Santa Ana, CA), Immunocult™-XF Expansion Medium (StemCell Technologies, Vancouver, Canada), TexMACS (Miltenyi Biotech, Bergisch Gladbach, Germany), CTS™ OpTmizer™ T Cell Expansion (Thermo Fisher Scientific, Waltham, MA), LymphoONE™ T-Cell Expansion Xeno-Free Medium (Takara Bio Inc., Shiga, Japan), Dulbecco's Modified Eagle's Medium (DMEM), Minimal Essential Medium (MEM), Basal Medium Eagle (BME), RPMI 1640, F-10, F-12, Minimal Essential Medium with alpha modification (aMEM), Glasgow's Minimal Essential Medium (G-MEM), or Iscove's Modified Dulbecco's Medium.

In particular embodiments, culture media includes a glutamine-containing serum-free medium, such as X-VIVO™15 (Lonza), AIM-V (Life Technologies) or Cellgro SCGM (Mediatech). Generally, commercially available serum-free media have a high glutamine concentration and will be diluted for use. If a corresponding serum-free medium without glutamine is used, glutamine may be added. Serum-free medias are also described in International Patent Publication Nos. WO2009023194, WO2008137641, WO2006017370, WO2001011011, WO2007071389, WO2007016366, WO2006045064, WO2003064598, WO2001011011, US Patent Publication Nos. US20050037492, US 20080113433, US 20080299540, U.S. Pat. Nos. 5,324,666, 6,162,643, 6,103,529, 6,048,728, 7,709,229 and European Patent Application No. EP2243827.

Serum replacements have been developed in attempts to minimize the effects of blood-derived serum on cell culture, as well as minimize the amount of animal protein used for culture of human cells.

For example, an animal-contaminant-free serum replacement for use with human cells can include a combination of insulin, transferrin and selenium. Additionally or alternatively, an animal-contaminant-free serum replacement can include human or recombinantly produced albumin, transferrin and insulin.

Examples of commercially available animal-contaminant-free serum replacement compositions include the premix of ITS (Insulin, Transferrin and Selenium) available from Invitrogen corporation (ITS, Invitrogen, Catalogue No. 51500-056); Serum replacement 3 (SR3; Sigma, Catalogue No. S2640) which includes human serum albumin, human transferring and human recombinant insulin and does not contain growth factors, steroid hormones, glucocorticoids, cell adhesion factors, detectable Ig and mitogens; KnockOut™ (Invitrogen, Carlsbad, Calif.) serum replacement [Catalogue numbers A10992-01, A10992-02, part Nos. 12618-012 or 12618-013] which contains only human-derived or human recombinant proteins.

In particular embodiments, the serum replacement includes KnockOut™ (Invitrogen) serum replacement.

According to some embodiments, the serum replacement is diluted in a 1 to 100 ratio in order to reach a x1 working concentration.

According to some embodiments, the concentration of the serum replacement in the culture medium is in the range of from 1% [volume/volume (v/v)] to 50% (v/v), e.g., from 5% (v/v) to 40% (v/v), e.g., from 5% (v/v) to 30% (v/v), e.g., from 10% (v/v) to 30% (v/v), e.g., from 10% (v/v) to 25% (v/v), e.g., from 10% (v/v) to 20% (v/v), e.g., 10% (v/v), e.g., 15% (v/v), e.g., 20% (v/v), e.g., 30% (v/v), e.g., 25% (v/v).

Culture media described herein can include one or more of: amino acids, sodium pyruvate, vitamins, serum or plasma, one or more hormones, and one or more cytokine(s), e.g., in amounts sufficient for the growth and expansion of T cells. The culture media can further include one or more antibiotics (e.g., any of the antibiotics described herein) and/or one or more anti-fungal agents (e.g., any of the anti-fungal agents described herein).

Exemplary culture media for culturing recombinant receptor-expressing T cells include (i) RPMI supplemented with non-essential amino acids, sodium pyruvate, and penicillin/streptomycin; (ii) RPMI with HEPES, 5-15% human serum, 1-3% L-Glutamine, 0.5-1.5% penicillin/streptomycin, and 0.25×10⁻⁴-0.75×10⁻⁴ M β-MercaptoEthanol; (iii) RPMI-1640 supplemented with 10% fetal bovine serum (FBS), 2 mM L-glutamine, 10 mM HEPES, 100 U/ml penicillin and 100 m/mL streptomycin; (iv) DMEM medium supplemented with 10% FBS, 2 mM L-glutamine, 10 mM HEPES, 100 U/ml penicillin and 100 m/mL streptomycin; and (v) X-Vivo 15 medium (Lonza, Walkersville, MD) supplemented with 5% human AB serum (Gemcell, West Sacramento, CA), 1% HEPES (Gibco, Grand Island, NY), 1% Pen-Strep (Gibco), 1% GlutaMax (Gibco), and 2% N-acetyl cysteine (Sigma-Aldrich, St. Louis, MO). T cell culture media are also commercially available from Hyclone (Logan, UT) and Stemcell Technologies (e.g., ImmunoCult™ (ImmC). CTL-Test™ Medium (ImmunoSpot®, Cellular Technology, Ltd; Cleveland, OH) may also be used. Additional T cell culture components that can be added to such culture media are described in more detail below.

Culture conditions can optionally include activation/expansion agents. In particular embodiments, activation/expansion agents are included in the culture media or are added to the culture media. In particular embodiments, activation/expansion agents include nutrients, amino acids, antibiotics, ions, and/or stimulatory factors, such as cytokines, chemokines, antigens, binding partners, fusion proteins, recombinant soluble receptors, and feeder cells. In particular embodiments, stimulatory factors include cytokines, antigens (e.g., multi-specific binding molecules), and feeder cells.

Generally, T cells are activated by contacting the cells a surface having attached thereto an agent that stimulates a CD3 TCR complex associated signal in the T cell and a ligand that stimulates a co-stimulatory protein on the surface of the T cell. In some examples, the T cell may be activated by contacting the T cell with an anti-CD3 antibody, or antigen-binding fragment thereof, or an anti-CD2 antibody immobilized on a surface, or by contacting the T cell with a protein kinase C activator (e.g., bryostatin) in combination with a calcium ionophore. A ligand that binds the co-stimulatory protein on the surface of a T cell can be used to active the co-stimulatory protein on the surface of a T cell. For example, a population of T cells can be contacted with an anti-CD3 antibody and an anti-CD28 antibody, under conditions appropriate for activating the T cells. An anti-CD3 antibody and an anti-CD28 antibody can be used to stimulate proliferation of either CD4+ T cells or CD8+ T cells. Additional methods for activating T cells are known in the art (Berg et al., Transplant Proc. 30(8):3975-3977, 1998; Haanen et al., J. Exp. Med. 190(9):1319-1328, 1999; and Garland et al., J. Immunol. Meth. 227(1-2):53-63, 1999). As described herein, in preferred embodiments, T cells are activated by a binding domain of a recombinant receptor that binds an immune activating epitope on an autologous cell type derived from a negative fraction of a cell isolation procedure.

In some examples, activation of the primary cytoplasmic signaling sequence and the co-stimulatory cytoplasmic signaling sequence signal in the T cell can be provided by different protocols. For example, the agents activating each signal may be in solution or coupled to a surface. When coupled to a surface, the agents may be coupled to the same surface (i.e., in “cis” formation) or to separate surfaces (i.e., in “trans” formation), In some examples, one agent may be coupled to a surface and the other agent in solution. In some examples, the agent providing activation of the co-stimulatory cytoplasmic signaling sequence is bound to a cell surface and the agent providing activation of the primary cytoplasmic signaling sequence is in solution or coupled to a surface. In certain embodiments, both agents can be in solution. In some examples, the agents may be in soluble form, and then cross-linked to a surface, such as a cell expressing Fc receptors or an antibody or other binding agent which will bind to the agents. See, e.g., U.S. Patent Application Publication Nos. 2004/0101519 and 2006/0034810 for artificial antigen presenting cells (aAPCs) that can be used for activating T cell in the present methods.

Activation/expansion agents can include cytokines. In particular embodiments, cytokines include interleukin (IL)-2, IL-4, IL-7, IL-10, IL-12, IL-15, IL-21, and/or IFN-γ. In particular embodiments, cytokines include IL-2, IL-4, IL-7, and IL-21.

An activation/expansion agent can include a multi-specific binding molecule, wherein the multi-specific binding molecule includes at least one binding domain that binds a recombinant receptor binding domain and at least one binding domain that binds an immune cell activating epitope.

Multi-specific binding molecules can be manufactured by any means known in the art. Multi-specific antibodies are antibodies with at least two binding domains. Multi-specific binding antibodies include bispecific antibodies, trispecific antibodies, and tetraspecific antibodies. A bispecific antibody binds at least two epitopes and a trispecific antibody binds at least 3 epitopes. The term “antibody”, unless otherwise indicated, includes antibodies, variants, derivatives, and fragments thereof. Bispecific antibodies can be prepared as full-length antibodies or antibody fragments (for example, F(ab′)2 bispecific antibodies). For example, WO 1996/016673 describes a bispecific ErbB2/Fc gamma RIII antibody; U.S. Pat. No. 5,837,234 describes a bispecific ErbB2/Fc gamma RI antibody; WO 1998/002463 describes a bispecific ErbB2/Fc alpha antibody; and U.S. Pat. No. 5,821,337 describes a bispecific ErbB2/CD3 antibody. Multi-specific binding molecules used within the current disclosure have at least one binding domain that binds an immune cell activating epitope within an nsPBMC sample, an nsPBMC sample/CD4+ sample, or a nsPBMC sample/CD8+ sample; and multi-specific binding molecules have at least one binding domain that binds a recombinant receptor binding domain expressed by a cell co-cultured with an nsPBMC sample, an nsPBMC sample/CD4+ sample, or an nsPBMC sample/CD8+ sample.

Some exemplary bispecific binding molecules have two heavy chains (each having three heavy chain CDRs, followed by (N-terminal to C-terminal) a CH1 domain, a hinge, a CH2 domain, and a CH3 domain). However, additional architectures are envisioned, including bispecific binding molecules in which light chain(s) associate with each heavy chain but do not (or minimally) contribute to antigen-binding specificity, or that can bind one or more of the epitopes bound by the heavy chain antigen-binding regions, or that can associate with each heavy chain and enable binding of one or both of the heavy chains to one or both epitopes.

Two binding domains can be linked through a linker to form a bispecific antibody. In particular embodiments, the two binding domains can bind the same epitope or different epitopes. The two binding domains can be linked through a linker. Commonly used flexible linkers include the Gly-Ser linkers as described elsewhere herein. Linkers can also include one or more antibody hinge regions and/or immunoglobulin heavy chain constant regions, such as CH3 alone or a CH2CH3 sequence. Additional examples of linkers can be found in Chen et al., Adv Drug Deliv Rev. 2013 Oct. 15; 65(10):1357-1369. Linkers can be flexible, rigid, or semi-rigid, depending on the desired functional domain presentation to a target.

Other forms of bispecific binding molecules include the single chain “Janusins” described in Traunecker et al. (Embo Journal, 10, 3655-3659, 1991) and BiTEs® (Amgen, Thousand Oaks, CA).

In particular embodiments, multi-specific binding molecules can be prepared using sequences or moieties that permit dimerization or multimerization. Such sequences include those derived from IgA or IgM, which permit formation of multimers in conjunction with the J-chain. Multi-specific binding molecules can be prepared using dimerization domains such as the Gal4 dimerization domain, biotin/avidin modifications, dimerization and docking domain (DDD), X-type four-helix bundle dimerization domain FcεRI α and β chain, FK-binding protein 12 (FKBP12) and FKBP12-Rapamycin Binding (FRB) domain of mTOR, receptor dimer pairs, zinc fingers, leucine zipper domain of Jun, the dimerization domain of Fos, a consensus sequence for a WW motif, the dimerization domain of the SH2B adapter protein (Nishi et al., Mol Cell Biol, 25:2607-2621, 2005), the SH3 domain of IB1 (Kristensen el al., EMBO J., 25:785-797, 2006), the PTB domain of human DOK-7 (Wagner et al., Cold Spring Harb Perspect Biol. 5: a008987, 2013), the PDZ-like domain of SATB1 (Galande et al., Mol Cell Biol. Aug; 21:5591-5604, 2001), the WD40 repeats of APAF (Jorgensen et al., 2009. PLOS One. 4(12):e8463), the PAS motif of the dioxin receptor (Pongratz et al., Mol Cell Biol, 18:4079-4088, 1998 and the EF hand motif of parvalbumin (Jamalian et al., Int J Proteomics, 2014:153712, 2014).

Examples of binding domains that bind immune cell activating epitopes are described elsewhere herein in relation to recombinant receptor binding domains (e.g., binding domains that bind CD19, CD20, etc.).

Activation/expansion agents can include feeder cells. In some embodiments, feeder cells are added to the culture media before adding the recombinant receptor-expressing T cells. In particular embodiments, the feeder cells are added to culture media containing recombinant receptor-expressing T cells.

The feeder cells are optionally from a different species as the recombinant receptor-expressing T cells they are supporting. For example, certain types of human cells, including stem cells, can be supported by primary cultures of mouse embryonic fibroblasts, or immortalized mouse embryonic fibroblasts. In another example, peripheral blood derived cells or transformed leukemia cells support the expansion and maturation of immune cells such as natural killer cells. In particular embodiments, feeder cells include nsPBMC. In particular embodiments, feeder cells include nsPBMC and CD4+ T cells. In particular embodiments, feeder cells include nsPBMC and CD8+ T cells. In particular embodiments, feeder cells can include EBV-transformed lymphoblastoid cells (LCL). In particular embodiments, feeder cells are added to a resulting population of cells such that at least 1, 2, 3, 5, 10, 20, or 40 feeder cell(s) are included for each T cell in the population.

The feeder cells may typically be inactivated when being co-cultured with other cells by irradiation or treatment with an anti-mitotic agent such as mitomycin to prevent them from outgrowing the cells they are supporting. In particular embodiments, irradiation includes gamma irradiation. In some embodiments, the feeder cells are irradiated with gamma rays in the range of 3000 to 10,000 rads to prevent cell division. In some embodiments, the feeder cells are irradiated with gamma rays in the range of 3000 to 3600 rads to prevent cell division. In some embodiments, the feeder cells are irradiated with gamma rays in the range of 6000 to 10,000 rads.

In certain examples, culture conditions include use of a G-REX® (Wilson Wolf Corporation, New Brighton, MN) gas-permeable flask including X-VIVO™ 15 (Lonza, Basel, Switzerland) +2% KnockOut™ Serum Replacement (KO) (Thermo Fisher Scientific, Waltham, MA) with 20 ng/ml IL4, 10 ng/ml IL7 and 20 ng/ml IL21. In certain examples, culture conditions include use of a G-REX® (Wilson Wolf Corporation, New Brighton, MN) gas-permeable flask including X-VIVO™ 15 (Lonza, Basel, Switzerland) +2% KnockOut™ Serum Replacement (KO) (Thermo Fisher Scientific, Waltham, MA) with 50 U/mL IL2, 20 ng/ml IL4, 10 ng/ml IL7 and 20 ng/ml IL21. These culture conditions can include 50 nM methotrexate (for example, added at day 3 of a culture). Media changes can occur every 3-4 days to refresh/replenish cytokine concentrations.

In particular embodiments, cells will be expanded within the culture media for 21 days. In particular embodiments, a sufficiently pure population of recombinant receptor-expressing T cells will be within the cell population at the end of one 21 day expansion period. In some instances, one may choose to submit the recombinant receptor-expressing T cell population to another expansion period to further increase the number or percentage of recombinant receptor-expressing T cells within the cell population. The second expansion period can duplicate the first expansion period in terms of ratio of feeder cell addbacks, concentration and identity of cytokines, and presence or absence of a multi-specific binding molecule. Subsequent expansion periods may also be conducted.

Methods for activating and culturing T cells are described in, e.g., U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

### (v) Ex Vivo Manufactured Cell Formulations

Formulations described herein can include ex vivo genetically modified cells manufactured according to a method disclosed herein. In particular embodiments, genetically-modified cells can be harvested from a culture medium and concentrated into a pharmaceutically-acceptable carrier in a therapeutically-effective amount. Genetically-modified cells are cells that contain genetic material that was artificially introduced so as to produce a desired characteristic (e.g., express a recombinant receptor).

The phrase “pharmaceutically acceptable” refer to those compounds, materials, and/or dosage forms which are, within the scope of sound medical judgment, suitable for use in contact with the tissues of human beings and animals without excessive toxicity, commensurate with a reasonable benefit/risk ratio. In certain instances, pharmaceutically-acceptable carriers have been approved by a relevant regulatory agency (e.g., the United States Food and Drug Administration (US FDA)).

Exemplary carriers include saline, buffered saline, physiological saline, water, Hanks' solution, Ringer's solution, Normosol-R (Abbott Labs), PLASMA-LYTE A® (Baxter Laboratories, Inc., Morton Grove, IL), and combinations thereof. In particular embodiments, carriers can be supplemented with human serum albumin (HSA) or other human serum components or fetal bovine serum. In particular embodiments, a carrier for infusion includes buffered saline with 5% HSA or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

In formulations disclosed herein, cells are generally in a volume of a liter or less, 500 ml or less, 250 ml or less or 100 ml or less. Hence the density of administered cells is typically greater than 10⁴ cells/ml, 10⁷ cells/ml, or 10⁸ cells/ml.

In particular embodiments, formulations can include one or more genetically modified cell types (e.g., modified T cells, NK cells, or stem cells) or genetically modified cells that express one or more recombinant receptor types. The different populations of genetically modified cells can be provided in different ratios.

The cell-based formulations disclosed herein can be prepared for administration by, e.g., injection, infusion, perfusion, or lavage. The formulations can further be formulated for bone marrow, intravenous, intradermal, intraarterial, intranodal, intralymphatic, intraperitoneal, intralesional, intraprostatic, intravaginal, intrarectal, intrathecal, intratumoral, intramuscular, intravesicular, and/or subcutaneous injection.

In some instances, it can be useful to cryopreserve modified cell formulations of the disclosure. “Cryopreserving,” refers to the preservation of cells by cooling to sub-zero temperatures, such as (typically) 77 K or −196° C. (the boiling point of liquid nitrogen). Cryoprotective agents are often used at sub-zero temperatures to ameliorate or prevent cell damage due to freezing at low temperatures or warming to room temperature. Cryoprotective agents and optimal cooling rates can protect against cell injury. Cryoprotective agents which can be used include dimethyl sulfoxide (DMSO) (Lovelock and Bishop, Nature, 1959; 183:1394-1395; Ashwood-Smith, Nature, 1961; 190:1204-1205), glycerol, polyvinylpyrrolidine (Rinfret, Ann. N.Y. Acad. Sci., 1960; 85:576), and polyethylene glycol (Sloviter and Ravdin, Nature, 1962; 196:48). In particular embodiments, the cooling rate is 1° to 3° C./minute. After at least two hours, the cells reach a temperature of −80° C. and can be placed directly into liquid nitrogen (−196° C.) for permanent storage such as in a long-term cryogenic storage vessel.

### (vi) Methods of Use

Methods disclosed herein include treating subjects (humans, non-human primates, veterinary animals (dogs, cats, reptiles, birds, etc.), livestock (horses, cattle, goats, pigs, chickens, etc.), and research animals (monkeys, rats, mice, fish, etc.) with formulations disclosed herein. Treating subjects includes delivering therapeutically effective amounts. Therapeutically effective amounts include those that provide effective amounts, prophylactic treatments and/or therapeutic treatments.

An “effective amount” is the amount of a formulation necessary to result in a desired physiological change in the subject. For example, an effective amount can provide an anti-cancer or anti-infection effect. Effective amounts are often administered for research purposes. Effective amounts disclosed herein can cause a statistically-significant effect in an animal model or in vitro assay relevant to the assessment of a cancer or infection's development or progression.

A “prophylactic treatment” includes a treatment administered to a subject who does not display signs or symptoms of a cancer or infection or displays only early signs or symptoms of a cancer or infection such that treatment is administered for the purpose of diminishing or decreasing the risk of developing the cancer or infection further. Thus, a prophylactic treatment functions as a preventative treatment against a cancer or infection. In particular embodiments, prophylactic treatments reduce, delay, or prevent metastasis from a primary cancer from occurring. In particular embodiments, prophylactic treatments reduce, delay, or prevent infection from a bacteria, virus, fungi, parasite, or arthropod.

A “therapeutic treatment” includes a treatment administered to a subject who displays symptoms or signs of a cancer or infection and is administered to the subject for the purpose of diminishing or eliminating those signs or symptoms of the cancer or infection. The therapeutic treatment can reduce, control, or eliminate the presence or activity of the cancer or infection and/or reduce control or eliminate side effects of the cancer or infection.

Function as an effective amount, prophylactic treatment or therapeutic treatment are not mutually exclusive, and in particular embodiments, administered dosages may accomplish more than one treatment type.

In particular embodiments, therapeutically effective amounts provide anti-cancer effects. Anti-cancer effects include a decrease in the number of cancer cells, decrease in the number of metastases, a decrease in tumor volume, an increase in life expectancy, induced chemo-or radiosensitivity in cancer cells, inhibited angiogenesis near cancer cells, inhibited cancer cell proliferation, inhibited tumor growth, prevented or reduced metastases, prolonged subject life, reduced cancer-associated pain, and/or reduced relapse or re-occurrence of cancer following treatment.

A “tumor” is a swelling or lesion formed by an abnormal growth of cells (called neoplastic cells or tumor cells). A “tumor cell” is an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be benign, pre-malignant or malignant.

Types of cancer that can be treated using ex vivo manufactured T cells include prostate cancer, breast cancer, stem cell cancer, ovarian cancer, mesothelioma, renal cell carcinoma melanoma, pancreatic cancer, lung cancer, HBV-induced hepatocellular carcinoma, and multiple myeloma. Further exemplary cancers that may be treated include medulloblastoma, oligodendroglioma, ovarian clear cell adenocarcinoma, ovarian endomethrioid adenocarcinoma, ovarian serous adenocarcinoma, pancreatic ductal adenocarcinoma, pancreatic endocrine tumor, malignant rhabdoid tumor, astrocytoma, atypical teratoid rhabdoid tumor, choroid plexus carcinoma, choroid plexus papilloma, ependymoma, glioblastoma, meningioma, neuroglial tumor, oligoastrocytoma, oligodendroglioma, pineoblastoma, carcinosarcoma, chordoma, extragonadal germ cell tumor, extrarenal rhabdoid tumor, schwannoma, skin squamous cell carcinoma, chondrosarcoma, clear cell sarcoma of soft tissue, Ewing sarcoma, gastrointestinal stromal tumor, osteosarcoma, rhabdomyosarcoma, epitheloid sarcoma, renal medullo carcinoma, diffuse large B-cell lymphoma, follicular lymphoma and not otherwise specified (NOS) sarcoma.

Acute myeloid leukemia (AML), blastic plasmacytoid dendritic cell neoplasm (BPDCN), myelodysplastic syndromes (MDS), natural killer cell lymphomas, hairy cell leukemia, acute lymphocytic leukemia (ALL; also known as acute lymphoblastic lymphoma), chronic myelocytic leukemia (CML), other leukemias, hematological cancers or tumors, Hodgkin's lymphoma (HL), B-cell HL, non-Hodgkin lymphoma (NHL), mantle cell lymphoma (MCL), T cell lymphoma, multiple myeloma (refractory, relapsed, etc.), systemic mastocytosis (SM), hypereosinophilic syndrome (HES), myelofibrosis, anemia, systemic lupus erythematosus (SLE), psoriasis, and systemic sclerosis (scleroderma) may also be treated with formulations disclosed herein.

In particular embodiments, therapeutically effective amounts provide anti-infection effects. Anti-infection effects include a reducing or preventing an infection pathogen (e.g. bacteria, virus, fungi, parasite, or arthropod) from infecting a cell, decreasing the number of infected cells, decreasing the volume of infected tissue, increasing lifespan, increasing life expectancy, reducing or eliminating infection-associated symptoms (e.g., fever, aches, vomiting, loss of appetite, weight loss, diarrhea, bloating, abdominal pain, skin rashes, coughing, and/or a runny nose).

Infections that can be treated by disclosed formulations include bacterial, viral, fungal, parasitic, and arthropod infections. In particular embodiments, the infections are chronic. In particular embodiments, bacterial infections can include infections caused by Staphylococcusspp., Streptococcus spp., Campylobacter jejuni, Clostridium botulinum, Clostridium difficile, Escherichia coli, Listeria monocytogenes, Salmonella, Vibrio, Chlamydia trachomatis, Neisseria gonorrhoeae, and Treponema pallidum. In particular embodiments, viral infections can include infections caused by rhinovirus, influenza virus, respiratory syncytial virus (RSV), coronavirus (e.g., MERS, SARS, SARS-COV-2), herpes simplex virus-1 (HSV-1), varicella-zoster virus (VZV), hepatitis A, norovirus, rotavirus, human papillomavirus (HPV), hepatitis B, human immunodeficiency virus (HIV), herpes simplex virus-2 (HSV-2), Epstein-Barr virus (EBV), West Nile virus (WNV), enterovirus, hepatitis C, human T-lymphotrophic virus-1 (HTLV-1), and Merkel cell polyomavirus (MCV). In particular embodiments, fungal infections can include infections caused by Trychophyton spp. and Candida spp. . . . In particular embodiments, parasitic infections can include infections caused by Giardia, toxoplasmosis, E. vermicularis, Trypanosoma cruzi, Echinococcosis, Cysticercosis, Toxocariasis, Trichomoniasis, and Amebiasis. In particular embodiments, arthropod infections can include infections spread by arthropods infected with viruses or bacteria, including California encephalitis, Chikungunya, dengue, Eastern equine encephalitis, Powassan, St. Louis encephalitis, West Nile, Yellow Fever, Zika, Lyme disease, and babesiosis.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of cancer or infection, stage of cancer or infection, previous or concurrent therapeutic interventions, idiopathy of the subject and route of administration.

Therapeutically effective amounts of cells to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly).

As indicated, the formulations disclosed herein can be administered by, for example, injection, infusion, perfusion, or lavage and can more particularly include administration through one or more of intra-bone marrow, intravenous, intradermal, intraarterial, intranodal, intralymphatic, intraperitoneal, intralesional, intraprostatic, intravaginal, intrarectal, topical, intrathecal, intratumoral, intramuscular, intravesicular, and/or subcutaneous administration.

### (vii) Exemplary Embodiments

1. A method including:
   obtaining CD8+ cells isolated from a sample;
   obtaining CD4+ cells isolated from the same sample;
   retaining a negative fraction of the sample following the CD8+ cell isolation and the CD4+ cell isolation;
   introducing a chimeric antigen receptor (CAR)-encoding genetic construct into the isolated CD8+ T cells, wherein the expressed CAR includes a binding domain that binds an immune activating epitope expressed by a cell within the negative fraction; and
   co-culturing the CAR-expressing CD8+ cells with the negative fraction within a cytokine-supplemented media.
2. A method including:
   obtaining CD8+ cells isolated from a sample;
   obtaining CD4+ cells isolated from the same sample;
   retaining a negative fraction of the sample following the CD8+ cell isolation and the CD4+ cell isolation;
   introducing a chimeric antigen receptor (CAR)-encoding genetic construct into the isolated CD8+ T cells, wherein the expressed CAR includes a binding domain that binds an immune activating epitope expressed by a cell within the negative fraction; and
   co-culturing the CAR-expressing CD8+ cells with the CD4+ cells and the negative fraction within a cytokine-supplemented media.
3. A method including:
   obtaining CD8+ cells isolated from a sample;
   obtaining CD4+ cells isolated from the same sample;
   retaining a negative fraction of the sample following the CD8+ cell isolation and the CD4+ cell isolation;
   introducing a chimeric antigen receptor (CAR)-encoding genetic construct into the isolated CD4+ T cells, wherein the expressed CAR includes a binding domain that binds an immune activating epitope expressed by a cell within the negative fraction; and
   co-culturing the CAR-expressing CD4+ cells with the CD8+ cells and the negative fraction within a cytokine-supplemented media.
4. A method including:
   obtaining T cells from a sample;
   retaining a negative fraction of cells from the sample after the obtaining;
   introducing a genetic construct encoding a recombinant receptor into the obtained T cells; and
   co-culturing the T cells with the introduced genetic construct with the negative fraction of cells within a cytokine-supplemented media,
   wherein the T cells with the introduced genetic construct express a binding domain that binds an immune cell activating epitope within the co-culture.
5. The method of embodiment 4, wherein the binding domain that binds the immune cell activating epitope within the co-culture is part of the recombinant receptor.
6. The method of embodiment 5, wherein the immune cell activating epitope is expressed by a cell within the negative fraction.
7. The method of any of embodiments 4-6, wherein the sample includes peripheral blood mononuclear cells (PBMCs).
8. The method of any of embodiments 4-7, wherein the obtaining T cells includes
   obtaining CD8+ cells from the sample; and
   obtaining CD4+ cells from the same sample.
9. The method of any of embodiments 4-7, wherein the obtaining T cells includes obtaining CD8+ cells from the sample.
10. The method of any of embodiments 4-7, wherein the obtaining T cells includes obtaining CD4+ cells from the same sample.
11. The method of any of embodiments 4-10, wherein the introducing the genetic construct includes electroporation.
12. The method of embodiment 11, wherein the electroporation includes electroporation of 2 million T cells.
13. The method of any of embodiments 4-10, wherein the introducing the genetic construct includes lentiviral transduction.
14. The method of any of embodiments 4-13, wherein the genetic construct includes a vector.
15. The method of embodiment 14, wherein the vector is a self-inactivating vector.
16. The method of any of embodiments 4-15, wherein the genetic construct includes a plasmid.
17. The method of any of embodiments 4-16, wherein the genetic construct includes two or more plasmids.
18. The method of any of embodiments 4-17, wherein the genetic construct includes a transposon.
19. The method of any of embodiments 4-18, further including introducing a transposase into the obtained T cells.
20. The method of any of embodiments 4-19, wherein a binding domain of the expressed recombinant receptor binds a cancer antigen, a viral antigen, a bacterial antigen, a fungal antigen, a parasitic antigen, or an arthropod antigen.
21. The method of embodiment 20, wherein the cancer antigen is carcinoembryonic antigen, prostate specific antigen, Prostate Stem Cell antigen, PSMA, Her2/neu, estrogen receptor, progesterone receptor, ephrinB2, CD19, CD20, CD22, CD23, CD123, CS-1, CE7, hB7H3, ROR1, mesothelin, c-Met, GD-2, MAGE A3 TCR, EGFR, EGFRvIII, EphA2, IL13Ra2, L1CAM, oaGD2, GD2, B7H3, CD33, FITC, VAR2CSA, MUC16, PD-L1, ERBB2, folate receptor, CD56; glypican-2, disialoganglioside, EpCam, L1-CAM, Lewis Y, WT-1, Tyrosinase related protein 1; GD2, B-cell maturation antigen, CD24, SV40 T, carboxy-anhydrase-IX; or CD133.
22. The method of embodiment 20, wherein the viral antigen is a coronavirus spike protein.
23. The method of any of embodiments 4-22, wherein the recombinant receptor is a chimeric antigen receptor (CAR).
24. The method of any of embodiments 4-23, wherein the recombinant receptor is an orthogonal CAR.
25. The method of any of embodiments 4-24, wherein the genetic construct is up to 20 kb in size.
26. The method of embodiment 25, wherein the genetic construct is up to 15 kb in size.
27. The method of any of embodiments 4-25, wherein the genetic construct is 3.4-20 kb in size.
28. The method of any of embodiments 4-27, wherein the genetic construct includes or encodes an insulator, a promoter, a transduction marker, and a selection cassette.
29. The method of any of embodiments 4-28, wherein the genetic construct includes at least two promoters driving expression of different transgenes.
30. The method of embodiment 29, wherein the different transgenes encode different expression products.
31. The method of embodiment 30, wherein the different expression products include a CAR and a CAR-enhancing transgene.
32. The method of embodiment 31, wherein the CAR-enhancing transgene is PD1 (A99L): MYD88, PD1 (A99L): CD28, PD1 (A99L): CD2, caSTAT5a, CCR2, dnSHP1, caBCL2, dnFADD, NOTCH1Ic, cFLIP.v3, dnSHP2, caHIF1a, dnDGKa, dnBLIMP1, dnTGFbRII, caAKT, dnNRDP1, or PCK1
33. The method of any of embodiments 28-32, wherein the promoter is an iSynPro promoter, an MND promoter, an EF1a long promoter, or an EF1a short promoter.
34. The method of any of embodiments 4-32, further including enriching for T cells with the introduced genetic construct.
35. The method of embodiment 34, wherein the enriching includes adding a selection agent to the cytokine-supplemented media during the co-culturing, wherein the genetic construct includes a selection cassette.
36. The method of embodiment 35, wherein the selection agent is methotrexate and the genetic construct includes dihydrofolate reductase (DHFR) gene or a DHFR double mutant gene.
37. The method of any of embodiments 4-36, wherein the co-culturing is within a gas-permeable vessel.
38. The method of embodiment 37, wherein the gas-permeable vessel supports cell expansion of concentrations of 10×10^{6/}mL or higher.
39. The method of any of embodiments 4-38, wherein the co-culturing is for 10-25 days.
40. The method of any of embodiments 4-39, wherein the co-culturing is for 14-21 days.
41. The method of any of embodiments 4-40, wherein the co-culturing is for 21 days
42. The method of any of embodiments 4-41, wherein the ratio of CD8+ cells to CD4+ cells to the negative fraction of cells during a co-culture is 1:1:2, 1:1:3, 1:1:4, 1:1:5, 1:1:6, 1:1:7, 1:1:8, 1:1:9, 1:1:10, 1:1:11, 1:1:12, 1:1:13, 1:1:14, 1:1:15, 1:1:16, 1:1:17, 1:1:18, 1:1:19, 1:1:20, 2:1:1, 3:1:2, or 3:2:4.
43. The method of any of embodiments 4-41, wherein the ratio of CD8+ cells to the negative fraction of cells during a co-culture is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 3:2, or 3:4.
44. The method of any of embodiments 4-41, wherein the ratio of CD4+ cells to the negative fraction of cells during a co-culture is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 3:2, or 3:4.
45. The method of any of embodiments 4-44, wherein the cytokine-supplemented media includes interleukin (IL)-4.
46. The method of any of embodiments 4-45, wherein the cytokine-supplemented media includes IL4, IL7, and IL21.
47. The method of any of embodiments 4-46, wherein the cytokine-supplemented media includes 5-40 ng/ml IL4, 5-20 ng/ml IL7, and 1-40 ng/ML IL21.
48. The method of any of embodiments 4-47, wherein the cytokine-supplemented media includes 20 ng/ml IL4, 10 ng/ML IL7, and 20 ng/ml IL21.
49. The method of any of embodiments 4-48, wherein the cytokine-supplemented media includes IL-2, IL4, IL7, and IL21.
50. The method of any of embodiments 4-49, wherein the cytokine-supplemented media includes 5-15 U/mL IL2, 5-40 ng/ml IL4, 5-20 ng/ml IL7, and 1-40 ng/ml IL21.
51. The method of any of embodiments 4-50, wherein the cytokine-supplemented media includes 50 U/mL IL2, 20 ng/ml IL4, 10 ng/ml IL7, and 20 ng/ml IL21.
52. The method of any of embodiments 4-51, wherein the cytokine-supplemented media is refreshed in the co-culture every 3-4 days.
53. The method of any of embodiments 6-52, wherein the immune cell activating epitope expressed by the cell within the negative fraction is naturally expressed by the cell.
54. The method of any of embodiments 6-52, wherein the immune cell activating epitope expressed by the cell within the negative fraction is expressed by the cell following introduction of a nucleic acid encoding the immune cell activating epitope into the cell.
55. The method of any of embodiments 4-54, wherein the immune cell activating epitope is a B cell ligand.
56. The method of embodiment 55, wherein the B cell ligand is CD19, CD20, CD34, CD38, CD45R, or CD33.
57. The method of cell ligand is CD19, CD20, CD34, CD38, CD45R, or CD34-54, wherein the immune cell activating epitope is a hapten.
58. The method of embodiment 57, wherein the hapten is anchored to a cell within the negative fraction.
59. The method of cell ligand is CD19, CD20, CD34, CD38, CD45R, or CD34-54, wherein the immune cell activating epitope is a multi-specific binding molecule
60. The method of embodiment 59, wherein the multi-specific binding molecule includes a first binding domain that binds the recombinant receptor and a second binding domain that binds a cell within the negative fraction.
61. The method of cell ligand is CD19, CD20, CD34, CD38, CD45R, or CD36-60, wherein the cell within the negative fraction is a B cell, a myeloid cell, or an NK cell.
62. The method of embodiments 60 or 61, wherein the second binding domain binds CD19, CD20, CD34, CD38, or CD45R on a B cell.
63. The method of any of embodiments 4-62, wherein the sample is a leukapheresis sample or a blood cone sample obtained from a single subject.
64. The method of any of embodiments 4-63, wherein the obtaining includes cell sorting.
65. A cell population formed according to a method of any of embodiments 4-64, wherein greater than 80% of the obtained T cells express the recombinant receptor.
66. The cell population of embodiment 65, wherein greater than 90% of the obtained T cells express the recombinant receptor.
67. The cell population of embodiments 65 or 66, wherein the recombinant receptor is a CAR.
68. The cell population of any of embodiments 65-67, wherein the recombinant receptor is an orthogonal CAR.
69. The cell population of any of embodiments 65-68, wherein the cell population includes at least 75 million cells.
70. The cell population of embodiment 69, wherein the at least 75 million cells are derived from a starting cell population of 2 million cells.
71. A cell population formed according to a method of any of embodiments 4-64, wherein the cell population has a naïve cell marker profile.
72. The cell population of embodiment 71, wherein the naïve cell marker profile is CD62L+/CD45RA+/CD27+/CD28+.
73. The cell population of embodiments 71 or 72, formulated for administration to a subject.

### (viii) Closing Paragraphs

The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157 (1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554, 101, the following hydrophilicity values have been assigned to amino acid residues: Arg (+3.0); Lys (+3.0); aspartate (+3.0+1); glutamate (+3.0+1); Ser (+0.3); Asn (+0.2); Gln (+0.2); Gly (0); Thr (−0.4); Pro (−0.5+1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 ug/ml salmon sperm blocking DNA; followed by washes at 50° C. with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Binds” refers to an association of a binding domain (of, for example, a CAR binding domain or a multispecific binding molecule) to its cognate binding molecule with an affinity or Ka (i.e., an equilibrium association constant of a particular binding interaction with units of 1/M) equal to or greater than 10⁵ M⁻¹, while not significantly associating with any other molecules or components in a relevant environment sample. Binding domains may be classified as “high affinity” or “low affinity”. In particular embodiments, “high affinity” binding domains refer to those binding domains with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Ka of up to 10⁷ M⁻¹, up to 10⁶ M⁻¹, up to 10⁵ M⁻¹. Alternatively, affinity may be defined as an equilibrium dissociation constant (Kd) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Ka (equilibrium association constant) for the cognate binding molecule that is higher than the reference binding domain or due to a Kd (dissociation constant) for the cognate binding molecule that is less than that of the reference binding domain, or due to an off-rate (Koff) for the cognate binding molecule that is less than that of the reference binding domain. A variety of assays are known for detecting binding domains that specifically bind a particular cognate binding molecule as well as determining binding affinities, such as Western blot, ELISA, and BIACORER analysis (see also, e.g., Scatchard, et al., 1949, Ann. N.Y. Acad. Sci. 51:660; and U.S. Pat. Nos. 5,283,173, 5,468,614, or the equivalent).

In particular embodiments, Kd can be characterized using BIAcore. For example, in particular embodiments, Kd can be measured using surface plasmon resonance assays using a BIACORE®-2000 or a BIACORE®-3000 (BIAcore, Inc., Piscataway, N.J.) at 25° C. with immobilized antigen CM5 chips at 10 response units (RU). Briefly, carboxymethylated dextran biosensor chips (CM5, BIACORE, Inc.) can be activated with N-ethyl-N′-(3-dimethylaminopropyl)-carbodiimide hydrochloride (EDC) and N-hydroxysuccinimide (NHS) according to the supplier's instructions. Antigen can be diluted with 10 mM sodium acetate, pH 4.8, to 5 μg/ml (0.2 μM) before injection at a flow rate of 5 μl/minute to achieve y 10 response units (RU) of coupled protein. Following the injection of antigen, 1 M ethanolamine can be injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of 25 μl/min. Association rates (kₒₙ) and dissociation rates (K_{off}) can be calculated using a simple one-to-one Langmuir binding model (BIACORE® Evaluation Software version 3.2) by simultaneously fitting the association and dissociation sensorgrams. The equilibrium dissociation constant (Kd) can be calculated as the ratio K_{off}/kₒₙ. See, e.g., Chen et al., J. Mol. Biol. 293:865-881, 1999. If the on-rate exceeds 106 M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to obtain a claimed effect according to a relevant experimental method described in the current disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

“A,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method comprising:
obtaining CD8+ cells isolated from a sample;
obtaining CD4+ cells isolated from the same sample;
retaining a negative fraction of the sample following the CD8+ cell isolation and the CD4+ cell isolation;
introducing a chimeric antigen receptor (CAR)-encoding genetic construct into the isolated CD8+ T cells, wherein the expressed CAR comprises a binding domain that binds an immune activating epitope expressed by a cell within the negative fraction; and
co-culturing the CAR-expressing CD8+ cells with the negative fraction within a cytokine-supplemented media.

2. A method comprising:
obtaining CD8+ cells isolated from a sample;
obtaining CD4+ cells isolated from the same sample;
retaining a negative fraction of the sample following the CD8+ cell isolation and the CD4+ cell isolation;
introducing a chimeric antigen receptor (CAR)-encoding genetic construct into the isolated CD8+ T cells, wherein the expressed CAR comprises a binding domain that binds an immune activating epitope expressed by a cell within the negative fraction; and
co-culturing the CAR-expressing CD8+ cells with the CD4+ cells and the negative fraction within a cytokine-supplemented media.

3. A method comprising:
obtaining CD8+ cells isolated from a sample;
obtaining CD4+ cells isolated from the same sample;
retaining a negative fraction of the sample following the CD8+ cell isolation and the CD4+ cell isolation;
introducing a chimeric antigen receptor (CAR)-encoding genetic construct into the isolated CD4+ T cells, wherein the expressed CAR comprises a binding domain that binds an immune activating epitope expressed by a cell within the negative fraction; and
co-culturing the CAR-expressing CD4+ cells with the CD8+ cells and the negative fraction within a cytokine-supplemented media.

4. A method comprising:
obtaining T cells from a sample;
retaining a negative fraction of cells from the sample after the obtaining;
introducing a genetic construct encoding a recombinant receptor into the obtained T cells; and
co-culturing the T cells with the introduced genetic construct with the negative fraction of cells within a cytokine-supplemented media,
wherein the T cells with the introduced genetic construct express a binding domain that binds an immune cell activating epitope within the co-culture.

5. The method of claim 4, wherein the binding domain that binds the immune cell activating epitope within the co-culture is part of the recombinant receptor.

6. The method of claim 5, wherein the immune cell activating epitope is expressed by a cell within the negative fraction.

7. The method of claim 4, wherein the sample comprises peripheral blood mononuclear cells (PBMCs).

8. The method of claim 4, wherein the obtaining T cells comprises
obtaining CD8+ cells from the sample; and
obtaining CD4+ cells from the same sample.

9. The method of claim 4, wherein the obtaining T cells comprises obtaining CD8+ cells from the sample.

10. The method of claim 4, wherein the obtaining T cells comprises obtaining CD4+ cells from the same sample.

11. The method of claim 4, wherein the introducing the genetic construct comprises electroporation.

12. The method of claim 11, wherein the electroporation comprises electroporation of 2 million T cells.

13. The method of claim 4, wherein the introducing the genetic construct comprises lentiviral transduction.

14. The method of claim 4, wherein the genetic construct comprises a vector.

15. The method of claim 14, wherein the vector is a self-inactivating vector.

16. The method of claim 4, wherein the genetic construct comprises a plasmid.

17. The method of claim 4, wherein the genetic construct comprises two or more plasmids.

18. The method of claim 4, wherein the genetic construct comprises a transposon.

19. The method of claim 4, further comprising introducing a transposase into the obtained T cells.

20. The method of claim 4, wherein a binding domain of the expressed recombinant receptor binds a cancer antigen, a viral antigen, a bacterial antigen, a fungal antigen, a parasitic antigen, or an arthropod antigen.

21. The method of claim 20, wherein the cancer antigen is carcinoembryonic antigen, prostate specific antigen, Prostate Stem Cell antigen, PSMA, Her2/neu, estrogen receptor, progesterone receptor, ephrinB2, CD19, CD20, CD22, CD23, CD123, CS-1, CE7, hB7H3, ROR1, mesothelin, c-Met, GD-2, MAGE A3 TCR, EGFR, EGFRvIII, EphA2, IL13Ra2, L1CAM, oaGD2, GD2, B7H3, CD33, FITC, VAR2CSA, MUC16, PD-L1, ERBB2, folate receptor, CD56; glypican-2, disialoganglioside, EpCam, L1-CAM, Lewis Y, WT-1, Tyrosinase related protein 1; GD2, B-cell maturation antigen, CD24, SV40 T, carboxy-anhydrase-IX; or CD133.

22. The method of claim 20, wherein the viral antigen is a coronavirus spike protein.

23. The method of claim 4, wherein the recombinant receptor is a chimeric antigen receptor (CAR).

24. The method of claim 4, wherein the recombinant receptor is an orthogonal CAR.

25. The method of claim 4, wherein the genetic construct is up to 20 kb in size.

26. The method of claim 25, wherein the genetic construct is up to 15 kb in size.

27. The method of claim 4, wherein the genetic construct is 3.4-20 kb in size.

28. The method of claim 4, wherein the genetic construct comprises or encodes an insulator, a promoter, a transduction marker, and a selection cassette.

29. The method of claim 4, wherein the genetic construct comprises at least two promoters driving expression of different transgenes.

30. The method of claim 29, wherein the different transgenes encode different expression products.

31. The method of claim 30, wherein the different expression products comprise a CAR and a CAR-enhancing transgene.

32. The method of claim 31, wherein the CAR-enhancing transgene is PD1 (A99L): MYD88, PD1(A99L): CD28, PD1 (A99L): CD2, caSTAT5a, CCR2, dnSHP1, caBCL2, dnFADD, NOTCH1Ic, cFLIP.v3, dnSHP2, caHIF1a, dnDGKa, dnBLIMP1, dnTGFbRII, caAKT, dnNRDP1, or PCK1.

33. The method of claim 28, wherein the promoter is an iSynPro promoter, an MND promoter, an EF1a long promoter, or an EF1a short promoter.

34. The method of claim 4, further comprising enriching for T cells with the introduced genetic construct.

35. The method of claim 34, wherein the enriching comprises adding a selection agent to the cytokine-supplemented media during the co-culturing, wherein the genetic construct comprises a selection cassette.

36. The method of claim 35, wherein the selection agent is methotrexate and the genetic construct comprises dihydrofolate reductase (DHFR) gene or a DHFR double mutant gene.

37. The method of claim 4, wherein the co-culturing is within a gas-permeable vessel.

38. The method of claim 37, wherein the gas-permeable vessel supports cell expansion of concentrations of 10×106/mL or higher.

39. The method of claim 4, wherein the co-culturing is for 10-25 days.

40. The method of claim 4, wherein the co-culturing is for 14-21 days.

41. The method of claim 4, wherein the co-culturing is for 21 days

42. The method of claim 4, wherein the ratio of CD8+ cells to CD4+ cells to the negative fraction of cells during a co-culture is 1:1:2, 1:1:3, 1:1:4, 1:1:5, 1:1:6, 1:1:7, 1:1:8, 1:1:9, 1:1:10, 1:1:11, 1:1:12, 1:1:13, 1:1:14, 1:1:15, 1:1:16, 1:1:17, 1:1:18, 1:1:19, 1:1:20, 2:1:1, 3:1:2, or 3:2:4.

43. The method of claim 4, wherein the ratio of CD8+ cells to the negative fraction of cells during a co-culture is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 3:2, or 3:4.

44. The method of claim 4, wherein the ratio of CD4+ cells to the negative fraction of cells during a co-culture is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 3:2, or 3:4.

45. The method of claim 4, wherein the cytokine-supplemented media comprises interleukin (IL)-4.

46. The method of claim 4, wherein the cytokine-supplemented media comprises IL4, IL7, and IL21.

47. The method of claim 4, wherein the cytokine-supplemented media comprises 5-40 ng/ml IL4, 5-20 ng/ml IL7, and 1-40 ng/ml IL21.

48. The method of claim 4, wherein the cytokine-supplemented media comprises 20 ng/ml IL4, 10 ng/ml IL7, and 20 ng/ml IL21.

49. The method of claim 4, wherein the cytokine-supplemented media comprises IL-2, IL4, IL7, and IL21.

50. The method of claim 4, wherein the cytokine-supplemented media comprises 5-15 U/mL IL2, 5-40 ng/ml IL4, 5-20 ng/ML IL7, and 1-40 ng/ML IL21.

51. The method of claim 4, wherein the cytokine-supplemented media comprises 50 U/ml IL2, 20 ng/ML IL4, 10 ng/ml IL7, and 20 ng/ML IL21.

52. The method of claim 4, wherein the cytokine-supplemented media is refreshed in the co-culture every 3-4 days.

53. The method of claim 6, wherein the immune cell activating epitope expressed by the cell within the negative fraction is naturally expressed by the cell.

54. The method of claim 6, wherein the immune cell activating epitope expressed by the cell within the negative fraction is expressed by the cell following introduction of a nucleic acid encoding the immune cell activating epitope into the cell.

55. The method of claim 4, wherein the immune cell activating epitope is a B cell ligand.

56. The method of claim 55, wherein the B cell ligand is CD19, CD20, CD34, CD38, CD45R, or CD33.

57. The method of claim 4, wherein the immune cell activating epitope is a hapten.

58. The method of claim 57, wherein the hapten is anchored to a cell within the negative fraction.

59. The method of claim 4, wherein the immune cell activating epitope is a multi-specific binding molecule

60. The method of claim 59, wherein the multi-specific binding molecule comprises a first binding domain that binds the recombinant receptor and a second binding domain that binds a cell within the negative fraction.

61. The method of claim 6, wherein the cell within the negative fraction is a B cell, a myeloid cell, or an NK cell.

62. The method of claim 60, wherein the second binding domain binds CD19, CD20, CD34, CD38, or CD45R on a B cell.

63. The method of claim 4, wherein the sample is a leukapheresis sample or a blood cone sample obtained from a single subject.

64. The method of claim 4, wherein the obtaining comprises cell sorting.

65. A cell population formed according to a method of claim 4, wherein greater than 80% of the obtained T cells express the recombinant receptor.

66. The cell population of claim 65, wherein greater than 90% of the obtained T cells express the recombinant receptor.

67. The cell population of claim 65, wherein the recombinant receptor is a CAR.

68. The cell population of claim 65, wherein the recombinant receptor is an orthogonal CAR.

69. The cell population of claim 65, wherein the cell population comprises at least 75 million cells.

70. The cell population of claim 69, wherein the at least 75 million cells are derived from a starting cell population of 2 million cells.

71. A cell population formed according to a method of claim 4, wherein the cell population has a naïve cell marker profile.

72. The cell population of claim 71, wherein the naïve cell marker profile is CD62L+/CD45RA+CD27+/CD28+.

73. The cell population of claim 71, formulated for administration to a subject.


SEQUENCE LISTING (68 sequences)

SEQ ID NO: 1 - DNA, 3571 bp, synthetic construct
  source 1..3571: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctggctagcgccgccaccatgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgggatccggcggcggagagggcagaggaagtcttctaacatgcggtgacgtggaggagaatcccggccctaggatggttggttcgctaaactgcatcgtcgctgtgtcccagaacatgggcatcggcaagaacggggacttcccctggccaccgctcaggaatgaatccagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgattaaggtaccgcggccgcttttccctctgccaaaaattatggggacatcatgaagccccttgagcatctgacttctggctaataaaggaaatttattttcattgcaatagtgtgttggaattttttgtgtctctcactcggaaggacataagg

SEQ ID NO: 2 - DNA, 7021 bp, synthetic construct
  source 1..7021: mol_type = other DNA
tcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggaggaaaaactcgagtagagtctagactctacattttgacacccccataatatttttccagaattaacagtataaattgcatctcttgttcaagagttccctatcactctctttaatcactactcacagtaacctcaactcctggctagccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaggaggaatggaggatgccaagaatattaagaaaggccctgccccattctaccctctggaagatggcactgctggtgagcaactgcacaaggccatgaagaggtatgccctggtccctggcaccattgccttcactgatgctcacattgaggtggacatcacctatgctgaatactttgagatgtctgtgaggctggcagaagccatgaaaagatatggactgaacaccaaccacaggattgtggtgtgctctgagaactctctccagttcttcatgcctgtgttaggagccctgttcattggagtggctgtggcccctgccaatgacatctacaatgagagagagctcctgaacagcatgggcatcagccagccaactgtggtctttgtgagcaagaagggcctgcaaaagatcctgaatgtgcagaagaagctgcccatcatccagaagatcatcatcatggacagcaagactgactaccagggcttccagagcatgtatacctttgtgaccagccacttaccccctggcttcaatgagtatgactttgtgcctgagagctttgacagggacaagaccattgctctgattatgaacagctctggctccactggactgcccaaaggtgtggctctgccccacagaactgcttgtgtgagattcagccatgccagagaccccatctttggcaaccagatcatccctgacactgccatcctgtctgtggttccattccatcatggctttggcatgttcacaacactggggtacctgatctgtggcttcagagtggtgctgatgtataggtttgaggaggagctgtttctgaggagcctacaagactacaagatccagtctgccctgctggtgcccactctgttcagcttctttgccaagagcaccctcattgacaagtatgacctgagcaacctgcatgagattgcctctggaggagcacccctgagcaaggaggtgggtgaggctgtggcaaagaggttccatctcccaggaatcagacagggctatggcctgactgagaccacctctgccatcctcatcacccctgaaggagatgacaagcctggtgctgtgggcaaggtggttcccttttttgaggccaaggtggtggacctggacactggcaagaccctgggagtgaaccagaggggtgagctgtgtgtgaggggtcccatgatcatgtctggctatgtgaacaaccctgaggccaccaatgccctgattgacaaggatggctggctgcactctggtgacattgcctactgggatgaggatgagcactttttcattgtggacaggctgaagagcctcatcaagtacaaaggctaccaagtggcacctgctgagctagagagcatcctgctccagcaccccaacatctttgatgctggtgtggctggcctgcctgatgatgatgctggagagctgcctgctgctgttgtggttctggagcatggaaagaccatgactgagaaggagattgtggactatgtggccagtcaggtgaccactgccaagaagctgaggggaggtgtggtgtttgtggatgaggtgccaaagggtctgactggcaagctggatgccagaaagatcagagagatcctgatcaaggccaagaagggtggcaaatgagcggccgcttttccctctgccaaaaattatggggacatcatgaagccccttgagcatctgacttctggctaataaaggaaatttattttcattgcaatagtgtgttggaattttttgtgtctctcactcggaaggacataaggtgagatctttcacaaaacaccagttatgctgagatctttcacaaaacaccagttatgcgctaatgcctttgggcctaacacgtgcctcacgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggttaattaagccgccaccatgcttctcctggtgacaagccttctgctctgtgagttaccacacccagcattcctcctgatcccagaggtgcagctggtcgaatccggaggaggactggtccagcccggccggagcctgagactgagctgtgccgcttccggattcacttttgacgattacgcaatgcattgggtgaggcaggcccctggcaaggggctggaatgggtctccggaatctcttggaacagtgggcgcattggatatgccgattctgtgaagggccgattcactatctctcgggacaacgctaaaaatagtctgtttctgcagatgaattccctgcgcgccgaggataccgccgtgtactattgcgcccgagaccagggctaccactactatgatagcgccgaacatgcattcgacatttggggacagggaactgtggtcaccgtgagctccggaggaggaggaagcggaggaggagggtccggaggcgggggatcacagagcgcactgacccagccacggagcgtgagcggatttcctgggcagtctgtcaccattagttgcacaggcaccacatcagacgatgtgagctggtaccagcagcacccagggaaggctccccagctgatgctgtatgacgtgtccaaaagaccttctggcgtcccacataggtttagtggaagccggagcggccgggcagccagtctgatcatttcagggctgcagacagaggacgaagctgattatttctgttctagttacgcaggcagatataactctgtgctgtttggcgggggaacaaagctgactgtcctggaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggggaagcggagctactaacttcagcctgctgaagcaggctggagacgtggaggagaaccctggacctatggttggttcgctaaactgcatcgtcgctgtgtcccagaacatgggcatcggcaagaacggggacttcccctggccaccgctcaggaatgaatccagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgatggcggcggagagggcagaggaagtcttctaacatgcggtgacgtggaggagaatcccggccctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggcactcctcttgctgctggtggtggccctggggatcggcctcttcatgtgacctgcaggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 3 - DNA, 6917 bp, synthetic construct
  source 1..6917: mol_type = other DNA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgctagccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaggaggaatggaggatgccaagaatattaagaaaggccctgccccattctaccctctggaagatggcactgctggtgagcaactgcacaaggccatgaagaggtatgccctggtccctggcaccattgccttcactgatgctcacattgaggtggacatcacctatgctgaatactttgagatgtctgtgaggctggcagaagccatgaaaagatatggactgaacaccaaccacaggattgtggtgtgctctgagaactctctccagttcttcatgcctgtgttaggagccctgttcattggagtggctgtggcccctgccaatgacatctacaatgagagagagctcctgaacagcatgggcatcagccagccaactgtggtctttgtgagcaagaagggcctgcaaaagatcctgaatgtgcagaagaagctgcccatcatccagaagatcatcatcatggacagcaagactgactaccagggcttccagagcatgtatacctttgtgaccagccacttaccccctggcttcaatgagtatgactttgtgcctgagagctttgacagggacaagaccattgctctgattatgaacagctctggctccactggactgcccaaaggtgtggctctgccccacagaactgcttgtgtgagattcagccatgccagagaccccatctttggcaaccagatcatccctgacactgccatcctgtctgtggttccattccatcatggctttggcatgttcacaacactggggtacctgatctgtggcttcagagtggtgctgatgtataggtttgaggaggagctgtttctgaggagcctacaagactacaagatccagtctgccctgctggtgcccactctgttcagcttctttgccaagagcaccctcattgacaagtatgacctgagcaacctgcatgagattgcctctggaggagcacccctgagcaaggaggtgggtgaggctgtggcaaagaggttccatctcccaggaatcagacagggctatggcctgactgagaccacctctgccatcctcatcacccctgaaggagatgacaagcctggtgctgtgggcaaggtggttcccttttttgaggccaaggtggtggacctggacactggcaagaccctgggagtgaaccagaggggtgagctgtgtgtgaggggtcccatgatcatgtctggctatgtgaacaaccctgaggccaccaatgccctgattgacaaggatggctggctgcactctggtgacattgcctactgggatgaggatgagcactttttcattgtggacaggctgaagagcctcatcaagtacaaaggctaccaagtggcacctgctgagctagagagcatcctgctccagcaccccaacatctttgatgctggtgtggctggcctgcctgatgatgatgctggagagctgcctgctgctgttgtggttctggagcatggaaagaccatgactgagaaggagattgtggactatgtggccagtcaggtgaccactgccaagaagctgaggggaggtgtggtgtttgtggatgaggtgccaaagggtctgactggcaagctggatgccagaaagatcagagagatcctgatcaaggccaagaagggtggcaaatgagcggccgcttttccctctgccaaaaattatggggacatcatgaagccccttgagcatctgacttctggctaataaaggaaatttattttcattgcaatagtgtgttggaattttttgtgtctctcactcggaaggacataaggtgagatctttcacaaaacaccagttatgctgagatctttcacaaaacaccagttatgcgctaatgcctttgggcctaacacgtgcctcacgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggttaattaagccgccaccatgcttctcctggtgacaagccttctgctctgtgagttaccacacccagcattcctcctgatcccagaggtgcagctggtcgaatccggaggaggactggtccagcccggccggagcctgagactgagctgtgccgcttccggattcacttttgacgattacgcaatgcattgggtgaggcaggcccctggcaaggggctggaatgggtctccggaatctcttggaacagtgggcgcattggatatgccgattctgtgaagggccgattcactatctctcgggacaacgctaaaaatagtctgtttctgcagatgaattccctgcgcgccgaggataccgccgtgtactattgcgcccgagaccagggctaccactactatgatagcgccgaacatgcattcgacatttggggacagggaactgtggtcaccgtgagctccggaggaggaggaagcggaggaggagggtccggaggcgggggatcacagagcgcactgacccagccacggagcgtgagcggatttcctgggcagtctgtcaccattagttgcacaggcaccacatcagacgatgtgagctggtaccagcagcacccagggaaggctccccagctgatgctgtatgacgtgtccaaaagaccttctggcgtcccacataggtttagtggaagccggagcggccgggcagccagtctgatcatttcagggctgcagacagaggacgaagctgattatttctgttctagttacgcaggcagatataactctgtgctgtttggcgggggaacaaagctgactgtcctggaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggggaagcggagctactaacttcagcctgctgaagcaggctggagacgtggaggagaaccctggacctatggttggttcgctaaactgcatcgtcgctgtgtcccagaacatgggcatcggcaagaacggggacttcccctggccaccgctcaggaatgaatccagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgatggcggcggagagggcagaggaagtcttctaacatgcggtgacgtggaggagaatcccggccctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggcactcctcttgctgctggtggtggccctggggatcggcctcttcatgtgacctgcaggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 4 - DNA, 6618 bp, synthetic construct
  source 1..6618: mol_type = other DNA
tcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggaggaaaaactcgagtagagtctagactctacattttgacacccccataatatttttccagaattaacagtataaattgcatctcttgttcaagagttccctatcactctctttaatcactactcacagtaacctcaactcctggctagcgccgccaccatgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaagctccagatcaaagagagcctgagagccgagctgagagtgaccgagagaagggccgaagtgcctaccgcccaccctagcccatctccaagacctgccggccagttccagacactggtcgtgggagtcgtgggcggactgctgggatctctggtgctgctcgtgtgggtgctggccgtgattggcggaggaatggctgctggcggacctggcgctggatctgctgctcctgtgtctagcaccagcagcctgcctctggccgccctgaatatgagagtgcggcggagactgagcctgttcctgaacgtgcggacacaggtggccgccgattggacagctctggccgaggaaatggacttcgagtacctggaaatccggcagctggaaacccaggccgaccctacaggacgcctgctggatgcttggcagggcagaccaggcgcttctgtggggagactgctggaactgctgaccaagctgggccgggacgacgtgctgctggaactgggccctagcatcgaagaggactgccagaagtacatcctgaagcagcagcaggaagaggccgagaagcctctgcaggtggcagccgtggatagcagcgtgccaagaacagctgagctggccggaatcaccaccctggacgatcctctgggccacatgcccgagagattcgacgccttcatctgctactgccccagcgacatccagttcgtgcaggaaatgatcagacagctggaacagaccaactaccggctgaagctgtgcgtgtccgaccgggatgtgctgcctggcacctgtgtgtggtctatcgccagcgagctgatcgagaagcggtgcagacggatggtcgtggtggtgtccgacgactacctgcagtccaaagagtgcgacttccagaccaagttcgccctgagcctgagccctggcgcccaccagaagagactgatccccatcaagtacaaggccatgaagaaagagttccccagcatcctgcggttcatcaccgtgtgcgactacaccaacccctgcaccaagtcctggttctggaccagactggccaaggccctgtctctgcctggcggcggagagggcagaggaagtcttctaacatgcggtgacgtggaggagaatcccggccctaggatgcttctcctggtgacaagccttctgctctgtgagttaccacacccagcattcctcctgatcccatgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagcccgttaacgggtggaggcagcggaggtggctccatcatctctgcggtggttggcattctgctggtcgtggtcttgggggtggtctttgggatcctcatctgagcggccgcttttccctctgccaaaaattatggggacatcatgaagccccttgagcatctgacttctggctaataaaggaaatttattttcattgcaatagtgtgttggaattttttgtgtctctcactcggaaggacataaggtgagatctttcacaaaacaccagttatgctgagatctttcacaaaacaccagttatgcgctaatgcctttgggcctaacacgtgcctcacgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggttaattaagccgccaccatgcttctcctggtgacaagccttctgctctgtgagttaccacacccagcattcctcctgatcccagaggtgcagctggtcgaatccggaggaggactggtccagcccggccggagcctgagactgagctgtgccgcttccggattcacttttgacgattacgcaatgcattgggtgaggcaggcccctggcaaggggctggaatgggtctccggaatctcttggaacagtgggcgcattggatatgccgattctgtgaagggccgattcactatctctcgggacaacgctaaaaatagtctgtttctgcagatgaattccctgcgcgccgaggataccgccgtgtactattgcgcccgagaccagggctaccactactatgatagcgccgaacatgcattcgacatttggggacagggaactgtggtcaccgtgagctccggaggaggaggaagcggaggaggagggtccggaggcgggggatcacagagcgcactgacccagccacggagcgtgagcggatttcctgggcagtctgtcaccattagttgcacaggcaccacatcagacgatgtgagctggtaccagcagcacccagggaaggctccccagctgatgctgtatgacgtgtccaaaagaccttctggcgtcccacataggtttagtggaagccggagcggccgggcagccagtctgatcatttcagggctgcagacagaggacgaagctgattatttctgttctagttacgcaggcagatataactctgtgctgtttggcgggggaacaaagctgactgtcctggaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggggaagcggagctactaacttcagcctgctgaagcaggctggagacgtggaggagaaccctggacctatggttggttcgctaaactgcatcgtcgctgtgtcccagaacatgggcatcggcaagaacggggacttcccctggccaccgctcaggaatgaatccagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgatggcggcggagagggcagaggaagtcttctaacatgcggtgacgtggaggagaatcccggccctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggcactcctcttgctgctggtggtggccctggggatcggcctcttcatgtgacctgcaggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 5 - protein, 26 aa, synthetic construct
  REGION 1..3: note = (GSG) residues can be at the 5' end of the peptide to improve cleavage efficiency
GSGATNFSLLKQAGDVEENPGPGASG

SEQ ID NO: 6 - protein, 21 aa, synthetic construct
  REGION 1..3: note = (GSG) residues can be at the 5' end of the peptide to improve cleavage efficiency
GSGEGRGSLLTCGDVEENPGP

SEQ ID NO: 7 - protein, 24 aa, synthetic construct
  REGION 1..3: note = (GSG) residues can be at the 5' end of the peptide to improve cleavage efficiency
GSGQCTNYALLKLAGDVESNPGPP

SEQ ID NO: 8 - protein, 25 aa, synthetic construct
  REGION 1..3: note = (GSG) residues can be at the 5' end of the peptide to improve cleavage efficiency
GSGVKQTLNFDLLKLAGDVESNPGP

SEQ ID NO: 9 - protein, 41 aa, Homo sapiens
VKRGRKKLLYIFKQPFMRPVQTTQEEDGCSCRFPEEEEGGC

SEQ ID NO: 10 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 26 aa, Homo sapiens
FWVLVVVGGVLACYSLLVTVAFIIFW

SEQ ID NO: 12 - DNA, 1641 bp, synthetic construct
  source 1..1641: mol_type = other DNA
atggaggatgccaagaatattaagaaaggccctgccccattctaccctctggaagatggcactgctggtgagcaactgcacaaggccatgaagaggtatgccctggtccctggcaccattgccttcactgatgctcacattgaggtggacatcacctatgctgaatactttgagatgtctgtgaggctggcagaagccatgaaaagatatggactgaacaccaaccacaggattgtggtgtgctctgagaactctctccagttcttcatgcctgtgttaggagccctgttcattggagtggctgtggcccctgccaatgacatctacaatgagagagagctcctgaacagcatgggcatcagccagccaactgtggtctttgtgagcaagaagggcctgcaaaagatcctgaatgtgcagaagaagctgcccatcatccagaagatcatcatcatggacagcaagactgactaccagggcttccagagcatgtatacctttgtgaccagccacttaccccctggcttcaatgagtatgactttgtgcctgagagctttgacagggacaagaccattgctctgattatgaacagctctggctccactggactgcccaaaggtgtggctctgccccacagaactgcttgtgtgagattcagccatgccagagaccccatctttggcaaccagatcatccctgacactgccatcctgtctgtggttccattccatcatggctttggcatgttcacaacactggggtacctgatctgtggcttcagagtggtgctgatgtataggtttgaggaggagctgtttctgaggagcctacaagactacaagatccagtctgccctgctggtgcccactctgttcagcttctttgccaagagcaccctcattgacaagtatgacctgagcaacctgcatgagattgcctctggaggagcacccctgagcaaggaggtgggtgaggctgtggcaaagaggttccatctcccaggaatcagacagggctatggcctgactgagaccacctctgccatcctcatcacccctgaaggagatgacaagcctggtgctgtgggcaaggtggttcccttttttgaggccaaggtggtggacctggacactggcaagaccctgggagtgaaccagaggggtgagctgtgtgtgaggggtcccatgatcatgtctggctatgtgaacaaccctgaggccaccaatgccctgattgacaaggatggctggctgcactctggtgacattgcctactgggatgaggatgagcactttttcattgtggacaggctgaagagcctcatcaagtacaaaggctaccaagtggcacctgctgagctagagagcatcctgctccagcaccccaacatctttgatgctggtgtggctggcctgcctgatgatgatgctggagagctgcctgctgctgttgtggttctggagcatggaaagaccatgactgagaaggagattgtggactatgtggccagtcaggtgaccactgccaagaagctgaggggaggtgtggtgtttgtggatgaggtgccaaagggtctgactggcaagctggatgccagaaagatcagagagatcctgatcaaggccaagaagggtggcaaa

SEQ ID NO: 13 - protein, 238 aa, synthetic construct
MSKGEELFTGVVPILVELDGDVNGHKFSVSGEGEGDATYGKLTLKFICTTGKLPVPWPTLVTTFTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 14 - protein, 591 aa, synthetic construct
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAVNSHGFPPEVEEQAAGTLPMSCAQESGMDRHPAACASARINV

SEQ ID NO: 15 - protein, 268 aa, synthetic construct
MLLLVTSLLLCELPHPAFLLIPEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSGGGGSGGGGSGGGGSQSALTQPRSVSGFPGQSVTISCTGTTSDDVSWYQQHPGKAPQLMLYDVSKRPSGVPHRFSGSRSGRAASLIISGLQTEDEADYFCSSYAGRYNSVLFGGGTKLTVL

SEQ ID NO: 16 - protein, 12 aa, synthetic construct
ESKYGPPCPPCP

SEQ ID NO: 17 - protein, 112 aa, Homo sapiens; identical to SEQ ID NO: 10

SEQ ID NO: 18 - protein, 42 aa, Homo sapiens
KRGRKKLLYIFKQPFMRPVQTTQEEDGCSCRFPEEEEGGCEL

SEQ ID NO: 19 - protein, 120 aa, synthetic construct
CHPECQPQNGSVTCFGPEADQCVACAHYKDPPFCVARCPSGVKPDLSYMPIWKFPDEEGACQPCPINCTHSCVDLDDKGCPAEQRASPLTGGGSGGGSIISAVVGILLVVVLGVVFGILI

SEQ ID NO: 20 - protein, 335 aa, synthetic construct
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 21 - protein, 187 aa, synthetic construct
MVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 22 - protein, 490 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKLQIKESLRAELRVTERRAEVPTAHPSPSPRPAGQFQTLVVGVVGGLLGSLVLLVWVLAVIGGGMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 23 - DNA, 1189 bp, Homo sapiens
  source 1..1189: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaa

SEQ ID NO: 24 - DNA, 181 bp, synthetic construct
  source 1..181: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 25 - DNA, 390 bp, synthetic construct
  source 1..390: mol_type = other DNA
ctgcttagggttaggcgttttgcgctgcttcgcgatcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggaggaaaaactcgagtagagtctagactctacattttgacacccccat

SEQ ID NO: 26 - DNA, 114 bp, synthetic construct
  source 1..114: mol_type = other DNA
acattttgacacccccataatatttttccagaattaacagtataaattgcatctcttgttcaagagttccctatcactctctttaatcactactcacagtaacctcaactcctg

SEQ ID NO: 27 - DNA, 204 bp, synthetic construct
  source 1..204: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatg

SEQ ID NO: 28 - DNA, 99 bp, synthetic construct
  source 1..99: mol_type = other DNA
gacctctggctaataaaggaaatttattttcattgcaatagtgtgttggaattttttgtgtctctcactcggaaggacatatgggagggcaaatcattt

SEQ ID NO: 29 - protein, 268 aa, synthetic construct; identical to SEQ ID NO: 15

SEQ ID NO: 30 - protein, 267 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 31 - protein, 266 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 32 - protein, 270 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKRT

SEQ ID NO: 33 - protein, 316 aa, synthetic construct
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITGQPMTFPPEALWVTVGLSVCLIALLVALAFVCWRKIKQSCEEENAGAEDQDGEGEGSKTALQPLKHSDSKEDDGQEIA

SEQ ID NO: 34 - protein, 264 aa, synthetic construct
MLLLVTSLLLCELPHPAFLLIPQVQLQQPGAELVKPGASVKLSCKASGYTFTGYWMHWVKQRPGHGLEWIGEINPSNGRTNYNERFKSKATLTVDKSSTTAFMQLSGLTSEDSAVYFCARDYYGTSYNFDYWGQGTTLTVSSGGGGSGGGGSGGGGSDIQMTQSSSSFSVSLGDRVTITCKANEDINNRLAWYQQTPGNSPRLLISGATNLVTGVPSRFSGSGSGKDYTLTITSLQAEDFATYYCQQYWSTPFTFGSGTELEIK

SEQ ID NO: 35 - protein, 265 aa, synthetic construct
MLLLVTSLLLCELPHPAFLLIPQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRT

SEQ ID NO: 36 - protein, 264 aa, synthetic construct
MLLLVTSLLLCELPHPAFLLIPDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGSTSGSGKPGSGEGSTKGDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKR

SEQ ID NO: 37 - protein, 259 aa, synthetic construct
MLLLVTSLLLCELPHPAFLLIPQVQLLESGGGLVQPGGSLRLSCAASGFTFSSYTMSWVRQAPGQALEWMGTISSRGTYTYYPDSVKGRFTISRDNAKNSLYLQMNSLRAEDTAVYYCAREAIFTHWGRGTLVTVSSGGGGSGGGGSGGGGSDIQLTQSPSSLSASVGDRVTITCKASQDINNYHSWYQQKPGQAPRLLIYRANRLVDGVPDRFSGSGYGTDFTLTINNIESEDAAYYFCLKYNVFPYTFGQGTKVEIK

SEQ ID NO: 38 - protein, 259 aa, synthetic construct
MLLLVTSLLLCELPHPAFLLIPQVQLLESGGGLVQPGGSLRLSCAASGFTFSSYTMSWVRQAPGQALEWMGTISSGGTYTYYPDSVKGRFTISRDNAKNSLYLQMNSLRAEDTAVYYCAREAIFTYWGRGTLVTVSSGGGGSGGGGSGGGGSDIQLTQSPSSLSASVGDRVTITCKASQDINNYLSWYQQKPGQAPRLLIYRANRLVDGVPDRFSGSGYGTDFTLTINNIESEDAAYYFCLKYDVFPYTFGQGTKVEIK

SEQ ID NO: 39 - protein, 273 aa, synthetic construct
MLLLVTSLLLCELPHPAFLLIPSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVS

SEQ ID NO: 40 - protein, 127 aa, synthetic construct
MLLLVTSLLLCELPHPAFLLIPSIVMTQTPKFLLVSAGDRVTITCKASQSVSNDVTWYQQKAGQSPKLLIYSASNRYSGVPDRFTGSGYGTAFTFTISTVQAEDLAVYFCQQDYSSFGGGTKLEIKR

SEQ ID NO: 41 - protein, 267 aa, synthetic construct
MLLLVTSLLLCELPHPAFLLIPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 42 - protein, 240 aa, synthetic construct
MLLLVTSLLLCELPHPAFLLIPDIQMTQSPSSLSASVGDRVTITCKASQDVGTAVAWYQQKPGKAPKLLIYSASYRSTGVPSRFSGSGSGTDFTLTISSLQPEDFATYYCQHHYSAPWTFGGGTKVEIKGGGGSGGGGSGGGGSEVQLVESGGGLVQPGGSLRLSCAASGFTFSRNGMSWVRQAPGKGLEWVATVSSGGSYIYYADSVKGRFTISRDNAKNSLYLQMNSLRAEDTAVYYC

SEQ ID NO: 43 - protein, 269 aa, synthetic construct
MLLLVTSLLLCELPHPAFLLIPEVQLVESGGGLVQPGGSLRLSCAASGFTFSRNGMSWVRQAPGKGLEWVATVSSGGSYIYYADSVKGRFTISRDNAKNSLYLQMNSLRAEDTAVYYCARQGTTALATRFFDVWGQGTLVTVSSGGGGSGGGGSGGGGSDIQMTQSPSSLSASVGDRVTITCKASQDVGTAVAWYQQKPGKAPKLLIYSASYRSTGVPSRFSGSGSGTDFTLTISSLQPEDFATYYCQHHYSAPWTFGGGTKVEIKESK

SEQ ID NO: 44 - protein, 264 aa, synthetic construct
MLLLVTSLLLCELPHPAFLLIPDIQMTQSPSSLSASVGDRVTITCTASLSVSSTYLHWYQQKPGKAPKLLIYSTSNLASGVPSRFSGSGSGTDFTLTISSLQPEDFATYYCHQYHRSPLTFGGGTKVEIKGGGGSGGGGSGGGGSEVQLVESGGGLVQPGGSLRLSCAASGFTFTKYGVHWVRQAPGKGLEWVAVKWAGGSTDYNSALMSRFTISRDNAKNSLYLQMNSLRAEDTAVYYCARDHRDAMDYWGQGTLVTVSSESK

SEQ ID NO: 45 - protein, 264 aa, synthetic construct
MLLLVTSLLLCELPHPAFLLIPEVQLVESGGGLVQPGGSLRLSCAASGFTFTKYGVHWVRQAPGKGLEWVAVKWAGGSTDYNSALMSRFTISRDNAKNSLYLQMNSLRAEDTAVYYCARDHRDAMDYWGQGTLVTVSSGGGGSGGGGSGGGGSDIQMTQSPSSLSASVGDRVTITCTASLSVSSTYLHWYQQKPGKAPKLLIYSTSNLASGVPSRFSGSGSGTDFTLTISSLQPEDFATYYCHQYHRSPLTFGGGTKVEIKESK

SEQ ID NO: 46 - protein, 269 aa, synthetic construct
MLLLVTSLLLCELPHPAFLLIPDVVMTQTPLSLPVSLGDQASISCRSSQSLLKNNGNTFLHWYLQKSGQSPKLLIYKVSNRLSGVPDRFSGSGSGTYFTLKISRVEAEDLGVYFCSQSTHIPYTFGGGTKLELKRGGGGSGGGGSGGGGSEVKLVESGGGLVLPGDSLRLSCATSEFTFTDYYMTWVRQPPRKALEWLGFIRNRANGYTTEYNPSVKGRFTISRDNSQSILYLQMNTLRTEDSATYYCARVSNWAFDYWGQGTTLTVSS

SEQ ID NO: 47 - protein, 270 aa, synthetic construct
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 48 - protein, 139 aa, synthetic construct
MLLLVTSLLLCELPHPAFLLIPQVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSS

SEQ ID NO: 49 - protein, 129 aa, synthetic construct
MLLLVTSLLLCELPHPAFLLIPDIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIK

SEQ ID NO: 50 - protein, 150 aa, synthetic construct
MLLLVTSLLLCELPHPAFLLIPQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGS

SEQ ID NO: 51 - protein, 448 aa, synthetic construct
MLLLVTSLLLCELPHPAFLLIPSLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKY

SEQ ID NO: 52 - protein, 133 aa, synthetic construct
MLLLVTSLLLCELPHPAFLLIPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRF

SEQ ID NO: 53 - protein, 5 aa, synthetic construct
  REPEAT 1..5: note = (GGGGS) can be repeated n times wherein n is an integer including 1, 2, 3, 4, 5, 6, 7, 8, 9, or 10
GGGGS

SEQ ID NO: 54 - protein, 9 aa, synthetic construct
  REPEAT 1..4: note = (GGGS) can be repeated n times wherein n is an integer including 1, 2, 3, 4, 5, 6, 7, 8, 9, or 10
  REPEAT 5..9: note = (GGGGS) can be repeated n times wherein n is an integer including 1, 2, 3, 4, 5, 6, 7, 8, 9, or 10
GGGSGGGGS

SEQ ID NO: 55 - protein, 7 aa, synthetic construct
  REPEAT 1..4: note = (GGGS) is repeated n times wherein n is an integer including 1, 2, 3, 4, 5, 6, 7, 8, 9, or 10
  REPEAT 5..7: note = (GGS) is repeated n times wherein n is an integer including 1, 2, 3, 4, 5, 6, 7, 8, 9, or 10
GGGSGGS

SEQ ID NO: 56 - protein, 9 aa, synthetic construct
  REPEAT 1..4: note = (GGGS) is repeated n times wherein n is an integer including 1, 2, 3, 4, 5, 6, 7, 8, 9, or 10
GGGSGGGGS

SEQ ID NO: 57 - protein, 20 aa, synthetic construct
GGGGSGGGGSGGGGSGGGGS

SEQ ID NO: 58 - protein, 15 aa, synthetic construct
GGGGSGGGGSGGGGS

SEQ ID NO: 59 - protein, 10 aa, synthetic construct
GGGGSGGGGS

SEQ ID NO: 60 - protein, 5 aa, synthetic construct
GGGGS

SEQ ID NO: 61 - protein, 8 aa, synthetic construct
GGGSGGGS

SEQ ID NO: 62 - protein, 4 aa, synthetic construct
GGGS

SEQ ID NO: 63 - protein, 6 aa, synthetic construct
GGSGGS

SEQ ID NO: 64 - protein, 11 aa, synthetic construct
GGSGGGSGGSG

SEQ ID NO: 65 - protein, 10 aa, synthetic construct
GGSGGGSGSG

SEQ ID NO: 66 - protein, 8 aa, synthetic construct
GGSGGGSG

SEQ ID NO: 67 - DNA, 544 bp, synthetic construct
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 68 - DNA, 245 bp, synthetic construct
  source 1..245: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctg